(19) United States
(12) Patent Application Publication
(10) Pub. No.: US 2024/0174719 A1
(43) Pub. Date: May 30, 2024

(54) PROTEINS AND USES THEREOF

(71) Applicant: James Cook University, Townsville, Queensland (AU)

(72) Inventors: Alexander Constantine Loukas, Townsville, Queensland (AU); Paul Robert Giacomin, Townsville, Queensland (AU); Stephanie Margaret Ryan, Townsville, Queensland (AU); Roland Ruscher, Townsville, Queensland (AU)

(73) Assignee: James Cook University, Townsville, Queensland (AU)

(21) Appl. No.: 18/550,807

(22) PCT Filed: Mar. 17, 2022
(86) PCT No.: PCT/AU2022/050234

§ 371 Date: Sep. 15, 2023

(30) Foreign Application Priority Data
Mar. 17, 2021 (AU) 2021900769
Mar. 3, 2022 (AU) 2022900508

(51) Int. Cl. C07K 14/00; A61P 37/06; C12N 15/63

(52) U.S. Cl. CPC C07K 14/001; A61P 37/06; C12N 15/63

(57) ABSTRACT

The present invention relates to proteins suitable for treating or preventing an inflammatory or autoimmune condition, pharmaceutical compositions comprising same, and methods of use comprising same.

### FIELD OFTHE INVENTION

The present invention relates to proteins suitable for treating or preventing an inflammatory or autoimmune condition, pharmaceutical compositions comprising same, and methods of use comprising same.

### CROSS REFERENCE TO EARLIER APPLICATIONS

This application claims priority to Australian provisional applications 2021900769 and 2022900508, the entire contents of each are herein incorporated by reference in their entirety.

### BACKGROUND OF THE INVENTION

Inflammation is a non-specific reaction by the body's immune system in response to a perceived injury or theat. Inflammation may also occur in response to an autoimmune condition, where body's immune system mistakenly attacks the body's own cells. Inflammatory bowel diseases (IBDs) and asthma are examples of inflammatory conditions which are the result of inappropriate immune responses.

IBD is a chronic idiopathic inflammation of the gastrointestinal tract and can be sub-classified into two immunologically distinct conditions: Crohn's disease (CD) and ulcerative colitis (UC). CD appears to be mainly mediated by activated T helper 1 and 17 cells (Th1/Th17) with an overproduction of pro-inflammatory cytokines interleukin (IL)-12 and interferon gamma (IFNγ), whereas UC is characterised by a mixed T helper 2 and 17 cell (Th2/Th17)-mediated pathology, with key roles for canonical Th2 cytokines such as IL-13. It has been shown that both UC and CD are expected to have a significant burden worldwide, with cases of UC expected to increase from 1,737,130 cases in 2016 to 1,867,305 cases in 2026 and diagnosed prevalent cases of CD are expected to increase from 1,339,543 cases in 2019 to 1,629,940 cases in 2029 (GlobalData, 2020).

Asthma has been widely described as an aberrant Th2 immune response characterized by airway eosinophilia, the production of IL-4, -5, -9, -10 and -13 cytokines, elevated antigen-specific immunoglobulin E IgE levels, increased mucus production and structural remodelling leading to airway obstruction and hyper-reactivity. It has been estimated that around 300 million people have asthma worldwide, and it is likely that by 2025 a further 100 million may be affected. According to World Health Organisation (WHO) estimates, there were 417,918 deaths due to asthma at the global level and 24.8 million disability-adjusted life years (DALYS) attributable to Asthma in 2016 (WHO, 2020).

Despite their different inflammatory phenotypes, both IBDs and asthma appear to evolve from an imbalance between effector T cells and regulatory T cells that results in an overwhelming inflammatory cascade. Moreover, both chronic conditions are of multifactorial aetiology, where genetic factors and environmental factors such as the gut microbiota all contribute to the pathogenesis.

Tumour necrosis factor (TNF) inhibitors revolutionised the therapy of I BDs and remain the first-line therapy for moderate-to-severe IBD patients not responding to conventional therapy. However, a consistent subset of patients (around 20%) do not respond to treatment, and a similar proportion of patients is likely to lose efficacy every year. Although these drugs are generally considered safe, adverse events are still not infrequent and some patients present contraindications. This creates a huge unmet need for agents that are efficacious in a long term and go beyond treating symptoms or blocking inflammation being triggered by the immune response.

Accordingly, there is a need for new or improved therapies that can be used for the treatment of inflammatory or autoimmune conditions such as IBDs and asthma.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present inventors have identified proteins that have been shown herein to display utility in the treatment of inflammatory conditions such as IBDs.

The present invention provides a protein comprising an amino acid sequence having at least about 70% sequence identity to any one of SEQ ID NOs:1-20, for example any one of SEQ ID NOs: 1-8, especially any one of SEQ ID NOs: 1, 4, 6, 8 and 11, more especially any one of SEQ ID NOs: 4, 8 and 11, even more especially any one of SEQ ID NOs: 4 and 8, or a biologically active fragment or variant thereof.

The amino acid sequence may be any one of more of SEQ ID NOs:1-20, that is, any one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. The amino acid sequence may preferably be any one or more of SEQ ID NOs:1-8, for example any one or more of SEQ ID NO:1, SEQ ID NO:2, SEQ ID NO:3, SEQ ID NO:4, SEQ ID NO:5, SEQ ID NO:6, SEQ ID NO:7 and SEQ ID NO:8. The amino acid sequence may preferably be any one or more of SEQ ID NO:1, SEQ ID NO:4, SEQ ID NO:6, SEQ ID NO:8 and SEQ ID NO:11, especially any one or more of SEQ ID: 4, SEQ ID NO: 8 and SEQ ID NO:11, more especially any one or both of SEQ ID NO:4 and SEQ ID NO:8.

The amino acid sequence of the protein may have at least about 80%, at least about 90%, at least about 95%, at least about 98%, or at least about 99% sequence identity, or other % identity described herein, to any one of SEQ ID NOs:1-20, for example any one of SEQ ID NOs: 1-8, especially any one of SEQ ID NOs: 1, 4, 6, 8 and 11, more especially any one of SEQ ID NOs: 4, 8 and 11, even more especially any one of SEQ ID NOs: 4 and 8.

In particular embodiments, the amino acid sequence of the protein is any one of SEQ ID NOs:1-20, for example any one of SEQ ID NOs: 1-8, especially any one of SEQ ID NOs: 1, 4, 6, 8 and 11, more especially any one of SEQ ID NOs: 4, 8 and 11, even more especially any one of SEQ ID NOs: 4 and 8.

The present invention also provides a protein comprising an amino acid sequence in which a total of up to about 250 amino acids have been substituted, inserted and/or deleted in any one of SEQ ID NOs:1-20, for example any one of SEQ ID NOs:1-8, especially any one of SEQ ID NOs: 1, 4, 6, 8 and 11, more especially any one of SEQ ID NOs: 4, 8 and 11, even more especially any one of SEQ ID NOs: 4 and 8, or a biologically active fragment or variant thereof.

The amino acid sequence of the protein may have a total of up to about 225, up to about 200, up to about 175, up to about 125, up to about 100, up to about 75, up to about 50, up to about 40, up to about 30, up to about 25, up to about 20, up to about 15 or up to about 10 amino acids, or other number described herein, which have been substituted, inserted and/or deleted in any one of SEQ ID NOs:1-20, for example SEQ ID NOs:1-8, especially any one of SEQ ID NOs: 1, 4, 6, 8 and 11, more especially any one of SEQ ID NOs:4, 8 and 11, even more especially any one of SEQ ID NOs:4 and 8.

The protein may be isolated, purified, substantially purified, enriched, synthetic or recombinant.

The present invention also provides a recombinant protein encoded by a nucleotide sequence having at least about 70% sequence identity with any one of SEQ ID NOs:21-53, for example any one of SEQ ID NOs: 21-28, especially any one of SEQ ID NOs: 21, 24, 26, 28 and 31, more especially any one of SEQ ID NOs: 24, 28 and 31, more especially any one of SEQ ID NOs: 24 and 28, or a biologically active fragment or variant thereof.

The nucleotide sequence may be any one of more of SEQ ID NOs:21-53, that is, any one or more of SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52 and SEQ ID NO:53. The nucleotide sequence may preferably be any one or more of SEQ ID NOs:21-28, for example any one or more of SEQ ID NO:21, SEQ ID NO:22, SEQ ID NO:23, SEQ ID NO:24, SEQ ID NO:25, SEQ ID NO:26, SEQ ID NO:27 and SEQ ID NO:28. The nucleotide sequence may preferably be any one or more of SEQ ID NO:21, SEQ ID NO: 24, SEQ ID NO:26, SEQ ID NO:28 and SEQ ID NO:31, especially any one of more of SEQ ID NO: 24, SEQ ID NO:28 and SEQ ID NO:31, more especially any one or more of SEQ ID NO:24 and SEQ ID NO:28.

In some embodiments, nucleotide sequence of the recombinant protein has at least about 80%, at least about 90%, at least about 95%, at least about 98%, or at least about 99% sequence identity to any one of SEQ ID NOs:21-53, for example SEQ ID NOs: 21-28, especially any one of SEQ ID NOs: 21, 24, 26, 28 and 31, more especially any one of SEQ ID NOs: 24, 28 and 31, even more especially any one of SEQ ID NOs: 24 and 28.

In particular embodiments, the nucleotide sequence is any one of SEQ ID NOs:21-53, especially SEQ ID NOs:21-28, especially any one of SEQ ID NOs: 21, 24, 26, 28 and 31, more especially any one of SEQ ID NOs: 24, 28 and 31, even more especially any one of SEQ ID NOs: 24 and 28.

In some embodiments, the protein is selected from a protein family having anti-inflammatory activity, especially SCP/TAPS, lysozyme and annexin protein families.

The present invention also provides a pharmaceutical composition comprising, consisting essentially of, or consisting of, the protein of the invention described herein or a biologically active fragment or variant thereof, and at least one pharmaceutically acceptable carrier.

The present invention also provides a method for treating or preventing an inflammatory or autoimmune condition comprising administering to a subject in need thereof the protein in the invention as described herein or a biologically active fragment or variant thereof, or the pharmaceutical composition described herein, thereby treating or preventing an inflammatory or autoimmune condition in the subject.

The present invention also provides a method for the treatment or prevention of an inflammatory or autoimmune condition in a subject comprising the steps of:
identifying a subject having an inflammatory or autoimmune condition; and
administering to the subject in need thereof a therapeutically effective amount of the protein of the invention as described herein or a biologically active fragment or variant thereof, or the pharmaceutical composition described herein,
   thereby treating or preventing an inflammatory or autoimmune condition in the subject.

The protein or the pharmaceutical composition as described herein may be administered, or formulated for administration, by any route described herein, preferably parenterally.

The present invention also provides a method for inhibiting progression of an inflammatory or autoimmune condition comprising administering to a subject in need thereof the protein of the invention described herein or a biologically active fragment or variant thereof, or the pharmaceutical composition described herein, thereby inhibiting progression of an inflammatory or autoimmune condition. The protein or pharmaceutical composition described herein may be administered, or formulated for administration, by any route described herein, preferably parenterally.

The present invention also provides a method for minimising, alleviating or ameliorating a symptom of an inflammatory or autoimmune condition comprising administering to a subject in need thereof the protein of the invention described herein or a biologically active fragment or variant thereof, or the pharmaceutical composition described herein, thereby minimising, alleviating or ameliorating a symptom of an inflammatory or autoimmune condition. The protein or pharmaceutical composition described herein may be administered, or formulated for administration, by any route described herein, preferably parenterally.

The present invention also provides the use of the protein of the invention as described herein, or a biologically active fragment or variant thereof, in the manufacture of a medicament for treating or preventing an inflammatory or autoimmune condition. The protein described herein may be administered, or formulated for administration, by any route described herein, preferably parenterally.

The present invention also provides the protein of the invention as described herein, or a biologically active fragment or variant thereof, for use in treating or preventing an inflammatory or autoimmune condition. The protein described herein may be administered, or formulated for administration, by any route described herein, preferably parenterally.

The inflammatory or autoimmune condition may be selected from an inflammatory bowel disease, irritable bowel syndrome, type 2 diabetes, asthma, rheumatoid arthritis, lupus, allergy and coeliac disease. In particular embodiments, the inflammatory or autoimmune condition is an inflammatory bowel disease, especially ulcerative colitis or Crohn's disease.

The present invention also provides a nucleic acid comprising a nucleotide sequence encoding the protein of the invention, or any other protein described herein.

The present invention also provides a vector or expression construct comprising a nucleic acid of the invention as described herein.

The present invention also provides a cell comprising a vector, expression construct or nucleic acid of the invention as described herein. The cell of the invention may be any cell described herein, preferably a mammalian cell.

The present invention also provides a method of producing a protein or a biologically active fragment or variant thereof comprising incubating the cell as described herein under conditions that allow it to express the protein or biologically active fragment or variant thereof.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Results showing effect of in vitro transcription/translation lysates containing different recombinant proteins of the invention in a trinitrobenzenesulfonic acid (TNBS) colitis model. FIG. 1A is a graph showing percentage body weight; data points represent mean±SEM and sample size (n=5 mice/group) and significance was determined by using a two-sample t-test to compare weights from day three against the weights from the control group and was adjusted for multiple testing denoted by *p≤0.05, and **p≤0.01. FIG. 1B shows photographs of colon lengths of the naïve (A), the negative control (B) and hookworm lysate 13 (containing recombinant protein SEQ ID NO:1) (C) groups. FIG. 10 is a graph showing colon length; All individual data points are represented, mean±SEM and sample size (n=5 mice/group) and significance was determined by statistical comparisons using a two-sample t-test against the control group and was adjusted for multiple testing denoted by *p≤0.05, **p≤0.01, and ***p≤0.001. FIG. 1D is a graph showing macroscopic score; all individual data points are represented, mean±SEM and sample size (n=5 mice/group) and significance was determined by statistical comparisons using a Mann-Whitney U test to compare each test group with the negative control group and adjusted for multiple testing denoted by *p≤0.05, and **p≤0.01. FIG. 1E is a graph showing combined clinical score; all individual data points are represented, mean±SEM and sample size (n=5 mice/group) and significance was determined by statistical comparisons assessed using Mann-Whitney U test against the control group and was adjusted for multiple testing denoted by *p≤0.05, **p≤0.01, ***p≤0.001.

FIG. 2. Graphs showing activity of recombinant proteins of the invention on secretion of human colon biopsy inflammatory cytokines stimulated with anti-CD3/CD28 beads. The recombinant proteins were expressed in mammalian cells and purified before adding to the biopsy cultures. Cytokines measured were tumour necrosis factor alpha (TNFα), interferon gamma (IFNγ) and interleukin-17A (IL-17A). Negative control: No stimulation with anti-CD3/CD28 beads, first bar. All other samples were stimulatred with anti-CD3/CD28 beads. Positive control: cyclosporin-A (CSA), third bar. Values were normalised and are presented as percentage of the PBS+beads condition. Bars represent the mean from 6-7 individual ulcerative colitis patients. Error bars: SEM. Statistical significance determined by one sample t test and denoted by *P≤0.05, **P≤0.01, ***P≤0.001, ****P≤0.0001.

FIG. 3. Graphs showing effect of cell-free lysates containing different recombinant proteins of the invention fused to eGFP in a trinitrobenzenesulfonic acid (TNBS) colitis model. BALB/c mice were injected intraperitoneally with 200 μl of protein-containing supernatant (protein concentration: 100 mg/ml) one day prior to intrarectal administration of trinitrobenzoic acid (TNBS) and were monitored daily for three days before euthanasia. FIG. 3A is a graph showing mean percent change of initial weight for 12 lysates compared to eGFP-only lysate control, and naïve mice (n=5/group). FIG. 3B is a graph showing clinical scores (mean and individual data points) for the 12 lysates combining faecal consistency, motility, piloerection and weight change at day 3. FIG. 3C is a graph showing macroscopic colon pathology scores (mean and individual data points) for the 12 lysates at day 3 necropsy. *p≤0.05, **p≤0.01, Mann-Whitney U-test compared to eGFP control. FIG. 3D is a graph showing combined statistical analysis of the efficacy of all 78 expressed proteins compared to respective eGFP control. The x-axis depicts the difference in mean of the combined z-scores of clinical outcomes (weight loss, colon length, clinical scores and macroscopic scores) between treatment and control (eGFP lysate vector), and the y-axis shows the p-value where p<0.01 or −log10>2 were regarded as significant. Proteins that achieved significant protection in both categories are depicted in red (top right quadrant), and proteins that did not show significant protection in both categories are depicted in blue.

FIG. 4. Graphs showing effect of recombinant proteins of the invention in a trinitrobenzenesulfonic acid (TNBS) colitis model. BALB/c mice were injected intraperitoneally with 20 μg of recombinant hookworm proteins generated in Expi293F human embryonic kidney cells one day prior to intrarectal administration of trinitrobenzoic acid (TNBS), and daily thereafter until termination. Control mice received either recombinant bovine serum albumin (BSA) expressed using the same production methods, or PBS vehicle. FIG. 4A is a graph showing mean percent change of initial weight. FIG. 4B is a graph showing clinical scores (mean and individual data points). FIG. 4C is a graph showing macroscopic scores of colon pathology (mean and individual data points). Data are combined from three individual experiments, where n=10-20 for each treatment group. *p≤0.05, **p≤0.01, ***p≤0.001, Mann-Whitney U-test compared to PBS control.

FIG. 5. Graphs showing activity of recombinant proteins of the invention on secretion of human colon biopsy inflammatory cytokines stimulated with anti-CD3/CD28 beads. Intraepithelial lymphocytes and lamina propria cells were isolated and combined from fresh colon biopsies collected from ulcerative colitis patients. Cells were treated with either PBS control, purified recombinant hookworm proteins or bovine serum albumin, generated in Expi293F cells, and then stimulated with aCD3/CD28 Dynabeads (TCR stimulation). Patient to patient variability was accounted for by normalizing all data to the PBS (vehicle) treated samples (100%, indicated by a dotted line). Ac-FAR-2 was used at 10 mg/ml, and all other proteins at 50 mg/ml. Positive control cells were treated with cyclosporin A (CSA) or were left unstimulated (PBS). For FIGS. 5A-C, cell-free supernatants were collected and analysed after 3 days of culture for TNF, IFNγ and IL-17A secretion. Values were normalised to PBS-treated and stimulated conditions for each respective patient (dotted line). Data are combined from two independent sample collection days, with each coloured symbol representing an individual patient sample (n=5-12 per treatment group) and the mean value for each treatment shown as a horizontal line.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The inventors have herein identified proteins that are useful for the suppression, prevention or treatment of an inflammatory or autoimmune condition. The proteins of the invention are secreted by hookworm parasites. Hookworms are known to manipulate the immune system to prevent inappropriate immune responses, which promotes their long-term survival and results in chronic infection, primarily due to the secretion of a variety of hookworm excretory/secretory (ES) proteins. The proteins of the invention are derivable or obtainable from hookworms and were specifically identified and selected by the inventors as described herein as proteins that may have utility in treating an inflammatory or autoimmune condition.

As shown in the Examples, the proteins of the invention have been shown to have utility in a number of well characterised models that are associated with activation of the inflammatory response, including colitis. These models are well characterised in the art to comprise episodes of acute and/or chronic inflammation. The inventors therefore recognise the application of the peptides of the invention for a wide range of conditions, disorders or diseases, including autoimmune diseases, which are accompanied by inflammation, of which those disclosed herein are just some examples.

As also shown in the Examples, the peptides of the invention are capable of reducing the secretion of pro-inflammatory cytokines from human immune cells. Non-limiting examples include IFN-γ, TNF-α and IL-17A.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “and/or”, e.g., “X and/or Y” shall be understood to mean either “X and Y” or “X or Y” and shall be taken to provide explicit support for both meanings or for either meaning.

As used herein, the term “about” refers to a quantity, value, dimension, size, or amount that varies by as much as 30%, 25%, 20%, 15% or 10% to a reference quantity, value, dimension, size, or amount.

As used herein, unless the context requires otherwise, the term “comprise”, and variations such as “comprises” and “comprising”, will be understood to imply the inclusion of a stated integer or step or group of integers or steps but not the exclusion of any other integer or step or group of integers or steps.

As used herein, the term “amino acid” or “amino acid residue” refers to a compound having an amino group and a carboxylic acid group. The amino acid may be a L- or D-isomer or mixtures thereof. The amino acid may have a naturally occurring side chain (see Table 1) or a non-proteinogenic side chain. The amino acid may also be further substituted in the α-position with a group selected from —C₁₋₆alkyl, —(CH₂)ₙCORₐ, —(CH₂)ₙR_{b} and —PO₃H, where n is an integer selected from 1 to 8, Rₐ is —OH, —NH₂, —NHC₁₋₃alkyl, —OC₁₋₃alkyl or —C₁₋₃alkyl and R_{b} is —OH, —SH, —SC₁₋₃alkyl, —OC₁₋₃alkyl, —NH₂, —NHC₁₋₃alkyl or —NHC(C═NH)NH₂ and where each alkyl group may be substituted with one or more groups selected from —OH, —NH₂, —NHC₁₋₃alkyl, —O₁₋₃alkyl, —SH, —SC₁₋₃alkyl, —CO₂H, —CO₂H, —CO₂C₁₋₃alkyl, —CONH₂ and —CONHC₁₋₃alkyl.

Amino acid structure and single and three letter abbreviations used throughout the specification are defined in Table 1, which lists the twenty proteinogenic naturally occurring amino acids which occur in proteins as L-isomers.

TABLE 1

| | |
|---|---|
| | (1) |
| 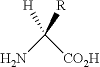 | |
| | (2) |
| 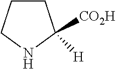 | |

| | Three-letter | One-letter | Structure of side chain |
|---|---|---|---|
| Amino Acid | Abbreviation | symbol | (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Glr | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Th | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

As used herein, the term “non-proteinogenic amino acid” refers to an amino acid having a side chain that does not occur in the naturally occurring L-α-amino acids recited in Table 1. Examples of non-proteinogenic amino acids and derivatives include, but are not limited to, norleucine, 4-aminobutyric acid, 4-amino-3-hydroxy-5-phenylpentanoic acid, 6-aminohexanoic acid, t-butylglycine, norvaline, phenylglycine, ornithine, citrulline, sarcosine, 4-amino-3-hydroxy-6-methylheptanoic acid, 2-thienyl alanine and/or D-isomers of natural amino acids

As used herein, the term “α-amino acid” refers to an amino acid that has a single carbon atom (the a-carbon atom) separating a carboxyl terminus (C-terminus) and an amino terminus (N-terminus). An α-amino acid includes naturally occurring and non-naturally occurring L-amino acids and their D-isomers and derivatives thereof such as salts or derivatives where functional groups are protected by suitable protecting groups. Unless otherwise stated, the term “amino acid” as used herein refers to an a-amino acid.

Those skilled in the art will appreciate that a peptide represents a series of two or more amino acids linked through a covalent bond formed between the carboxyl group of one amino acid and the amino group of another amino acid (i.e. the so-called peptide bond).

As used herein, the term “protein” or “polypeptide” will be understood to encompass a single polypeptide chain, i.e., a series of contiguous amino acids, such as a polymer, linked by peptide bonds or a series of polypeptide chains covalently or non-covalently linked to one another (i.e., a protein or polypeptide complex). For example, the series of polypeptide chains may be covalently linked by a suitable chemical or disulphide bond. Examples of non-covalent bonds include hydrogen bond, ionic bonds, Van der Waals forces and hydrophobic interactions. The protein or polypeptide may include amino acid residues and variants and synthetic analogues of the same. Thus, these terms encompass amino acid polymers in which one or more amino acid residues are synthetic non-proteinogenic amino acids, such as chemical analogues of corresponding naturally-occurring amino acids, as well polymers containing only naturally-occurring amino acids. These terms to not exclude, for example, glycosylations, acetylations, phosphorylations and the like. Included within the definition are, for example, proteins or polypeptides containing one or more analogs of an amino acid including, for example, unnatural amino acids or polypeptides with substituted linkages.

As used herein, the term “isolated” is intended to refer to molecules that are identified and separated from the form or setting in which it is found in nature. Accordingly, an “isolated protein” is other than the form or setting in which the protein is found in nature and an “isolated nucleic acid” is other than the form or setting in which the nucleic acid is found in nature.

As used herein, the term “purified” in the context of the protein of the invention will be understood to mean separated or isolated from a complex mixture such as a cell.

As used herein, the term “substantially purified” in the context of the protein of the invention means the protein is substantially free of contaminating agents, for example at least about 70%, at least about 75%, at least about 80%, at least about 85%, at least about 90%, at least about 65%, at least about 96%, at least about 97%, at least about 98%, or at least about 99% free of contaminating agents.

As used herein, the term “synthetic” in the context of the protein of the invention will be understood to refer to a protein produced by non-natural synthetic means, for example by chemical synthesis or artificial biological synthesis.

As used herein, the term “recombinant” will be understood to mean the product of artificial genetic recombination. Accordingly, the term “recombinant protein” is intended to encompass a protein expressed by artificial recombinant means when it is in an expression system or cell, for example in which it is expressed.

As used herein the term “derived from” will be understood to indicate that a specified integer may be obtained from a particular source albeit not necessarily directly from that source.

Additional definitions also are provided herein.

### Proteins

The present invention relates to proteins derivable or obtainable from hookworms including but not limited to Ancylostoma caninum, which may be useful for treating or preventing an inflammatory condition.

Accordingly, in one aspect, the present invention provides a protein comprising an amino acid sequence having at least 70% sequence identity to any one of SEQ ID NOs:1-20, or a biologically active fragment or variant thereof. SEQ ID NOs: 1-20 are set out in Table 2.

TABLE 2

| SEQ | | | | Amino acid sequence |
|---|---|---|---|---|
| ID | Lysate No | Family | Gene name | of protein |
| 1 | 13 | SCP/TAPS | ANCCAN_22177 | QAPDYQCWNFGQTNEIRHIYLSE |
| | | | | VNKLRQPIGEGKAPNKDGMCPQ |
| | | | | GKNIYKLFWDCILENYAQEAVDK |
| | | | | CGAKPTVPAELSMVWNETTLTTC |
| | | | | NPTPSFKKQVNDWWDEVKRVGL |
| | | | | GTDAAFKPGLENFAVLANGLATRI |
| | | | | GCAQKNCNGKLQMACVVYGKAA |
| | | | | SNAGQAIYEVGAGCSGDQDCTT |
| | | | | YAGSKCLTKKKFCVAGYPTSAVV |
| | | | | VPPTVGTTEAMTSSTQSTSTSTS |
| | | | | TSTQPEKTTTSEKPSTSATGTSNI |
| | | | | CPHSPPDANKDLAREELLKVHNE |
| | | | | KRAAIAKGEVKMGNGKMARRCP |
| | | | | RMKKFVRYNCTLEVAAYEKAKTC |
| | | | | PTADPNIENVSCFLSNARNKKLAA |
| | | | | REAMDAWFGEISKSNAHMEQAT |
| | | | | GSQNLLLPALNIRQFARMVWDRN |
| | | | | AEMGCGIAECKGSHKYVVVCQY |
| | | | | GQGVGKYGDTIYMMGPTONQCG |
| | | | | GPSGGRCTDGAFCP |
| 2 | 16 | SCP/TAPS | ANCCAN_12564 | QNAYCDGGDLLGAEIFEYILDPVN |
| | | | | EHRTTLVDGTQKNGLSGQNLPPA |
| | | | | KGMTRLDWDCDLEKEAKKIVTSH |
| | | | | CFDYVDEPPVMNNRAQVFLSAYA |
| | | | | DGLPNSPVVMQQLFAQALAEIDT |
| | | | | TALEGVTPGATNVIYKSGSVENLL |
| | | | | NYFNLMRPANTRMGCAWTKCTN |
| | | | | VDGDEFINVYCLLNSQQLKDGDVI |
| | | | | YNVGTAGVCSDCPTGTSCDRNT |
| | | | | KLCVRTTSVPTTGPAATTTPSTTT |
| | | | | TTPAPAQTTTTPPTSPEANFPSG |
| | | | | DSSRCSHPLASRMTNTLRSEYER |
| | | | | LHNFRRGLLATGQIPRKDGKHLP |
| | | | | TASNMQKMTYDCTLEEGAIQYAS |
| | | | | TCPTRLSELSSRPNVGENFKTFP |
| | | | | ATRFTFDTAAKKSVTEWWKVIRD |
| | | | | VNFFEKVVVFRPFHDGHPISSFT |
| | | | | QMAWATSNRLGCSIVNCSTSNR |
| | | | | YVGVCRYTQRGNIVNNNVYEVGA |
| | | | | VCSACPSGMTSCNRDEGLCF |
| 3 | 23 | SCP/TAPS | ANCCAN_12569 | MSFTTWLQQPHGGGGGGRCAG |
| | | | | SATADRMTDTLRDEFERLHNFRR |
| | | | | GLLATGQIPRKDGKYLPKASNMR |
| | | | | KIIYDCALEEGAIQYASQCPTTHS |
| | | | | EQSSRPNVGENFKTFPATRFTFD |
| | | | | TAAKKSVTEWWKPIRDVNYFEKV |
| | | | | VVFRPFHDGAPISSFTQMGWALS |
| | | | | HQVGCSIVMCSASNRYVGVCRY |
| | | | | SPRGNIVDNNVYMVGNPCSACH |
| | | | | SGATTCSSAEGLCF |
| 4 | 80 | Annexin | ANCCAN_07727 | MNSPYATIVDNQDFHPALYAEKID |
| | | superfamily | | RALRAGDKDTIVKVLTSISNNQRQ |
| | | | | QLREPYKIKYGKDIIAALDKKFSG |
| | | | | DLERTIFALMDTPLDYDVKQLKQA |
| | | | | MKGLGTDEAVLIEILCSRTPDQMA |
| | | | | AIRIAYEREYKTPLEKDVASDTSG |
| | | | | EFKDLLVALVTGSRDGSRNTNDQ |
| | | | | EAKDDAVRLYADGKAKLVGKDSP |
| | | | | SHFLKILATQNQYQLRKVFAAFAD |
| | | | | LSGSTIEKAIEKEFSGDLQKSYLTI |
| | | | | VQAASDKQKFFARQLYNSMKGL |
| | | | | GTRDNDLIRVLVTRSEVDLELIKQ |
| | | | | EFQALYNKPLEDMIKGDTSGQYR |
| | | | | DALLAIVVGNRM |
| 5 | 85 | SCP/TAPS | ANCCAN_00478 | NSMRCGNNGMTDEARQKFLDVH |
| | | | (Ac-ASP-2) | NSYRSMVAKGQAKDAISGNAPKA |
| | | | | AKMKKMIYDCNVESTAMQNAKK |
| | | | | CVFAHSHRKGVGENIWMSTARQ |
| | | | | MDKAQAAQQASDGWFSELAKYG |
| | | | | VGQENKLTTQLWNRGVMIGHYT |
| | | | | QMVWQESYKLGCYVEWCSSMT |
| | | | | YGVCQYSPQGNMMNSLIYEKGN |
| | | | | PCTKDSDCGSNASCSAGEALCV |
| | | | | VRG |
| 6 | 2 | Lysozyme- | ANCCAN_07322 | KAIAPEGVVKAAAGYAYAVDLEV |
| | | like | | PVPLSGFNCMKKYGYKTVFIRAY |
| | | | | DPTGAGRFDTNAVNNIRNANQAG |
| | | | | LGTEAYMTPQLHSNKRGSQQFR |
| | | | | ELYEGLRNAKIQVRTVWVQVTSP |
| | | | | VNWGPNTQANIYFINDIVSMAKYY |
| | | | | GVTIGFYTNVYDWNQITKGANVE |
| | | | | GAMLWYWNVNGAGVNGETPAN |
| | | | | FDDFRPFAKFTKPTVKQFGQVEH |
| | | | | LCGVTVNRDVYTLNNSINLVSSKA |
| | | | | EKLDGELIVGTLGGNQMSGLVQS |
| | | | | V |
| 7 | 22 | SCP/TAPS | ANCCAN_26187 | TTRFNCRNSLISDEWRQYVLDEL |
| | | | | NDYRRSLARGEVYDATKNFLPKA |
| | | | | KDMNELKWDCNLEEVAYKSLQSL |
| | | | | MSTCDVPLFSISRPYQTMQKRFK |
| | | | | RKECNITSQAKVLLYGWWKEIKN |
| | | | | TTVKYVNGNPSVPTGLEFIVMATA |
| | | | | TTTGFACTYEFCNNMPRGIIACIY |
| | | | | DDKTTDVSVANRIIYQATQNTQEI |
| | | | | CSSCSKNCIKSLCNVPHTPVTLPT |
| | | | | TCSDDKLTQDSNNAALWMHNYY |
| | | | | RKLLASGWAKDKKSKSGYALTAK |
| | | | | KMLKLEYDCAPAAGGISNGATKT |
| | | | | YDLIKDCPKSGNPTPSNGYSMNF |
| | | | | LRLKNHTISEQEALEQAIKKWWG |
| | | | | VLETKGLGSENTFSDNSEIASFAK |
| | | | | MAHDGTEKLACAVQNCKQNGET |
| | | | | LVACQYSPAIQDGDKIYETGKVC |
| | | | | GGCKALSKKCSDPAGLCE |
| 8 | 70 | TTR-52 | ANCCAN_08034 | KMQNVTVKGTTICNKKRMADVTV |
| | | superfamily | | ELWERDTLDPNDLLDSKKTSREG |
| | | | | EFLVKGGQNEVGSIEPFLRITHTC |
| | | | | NVKKPGCKRITEFDIPKSKIDTVY |
| | | | | DMTYVTLDIISAVDKEKC |
| 9 | 19 | SCP/TAPS | ANCCAN_07062 | PNYQCWNFGQQDEIRRDYLVEV |
| | | | | NKLRETIATGTAPSKDGRKCPQG |
| | | | | KNIYKLNWDCILENYAQKAADEC |
| | | | | AEPKTVPKKLSFVYNATTLTTCNP |
| | | | | IPDIKKQVNNWWSEVERVGLAKN |
| | | | | AAFKPKLRNFAVLANGLATRIGCA |
| | | | | QKNCNGVLHMVCVVYGKAASST |
| | | | | RQAIYEVGAGCEGNYQCTTYKES |
| | | | | KCLTKRKLCEAGYPTSEAVVPPT |
| | | | | VSTSKPTITSTEATQPEPQPPKPE |
| | | | | PPKPEPPKPETTKPPVTSEHCPK |
| | | | | QEHMNTDLAREEFVNLHNQARA |
| | | | | TIAKGGVKMGDGKMSRPCPRMK |
| | | | | KFDIKNYDCKLEEVAYATAAKCQT |
| | | | | EVYVQNENWFMASTKATNRKLA |
| | | | | AREAVEHWYNEIKSGHMEQKTG |
| | | | | SQNLLMPSFKIDHFARMVWDTNT |
| | | | | QIGCAAHKCGKIYHVVCRYGPKV |
| | | | | GKYGDTIYMMGPTCNQCQGRCI |
| | | | | EGAFCP |
| 10 | 73 | Tissue MET | ANCCAN_13497 | CKCEKKPRPPLEKLLCQSQFVTH |
| | | inhibitor | (Ac-TIMP-1) | AKVTKKRIDGYFIYYDLEHKEVYK |
| | | | | PKDRSIPIELFSWREKENCGVPDL |
| | | | | EEGKEYLIGGKVTDYGDGDLVISV |
| | | | | SRCDLLRNWTDVSGEEKKLLGTF |
| | | | | KCENQS |
| 11 | 79 | Fatty acid | ANCCAN_10127 | APNVEVNKFEDIPEQYRELIPKEV |
| | | retinol | (Ac-FAR-2) | ADHLKAITDDEKKILKEVLKDYAK |
| | | binding | | YKDENEYLAVLKEKSPSLHEKAK |
| | | protein | | KFHEFIKAKVDALGDEPKAFVKKV |
| | | | | IAAARKLHAELLAGNKPSLEELKN |
| | | | | TVKTYMAEFNALSPAAKEDLKKN |
| | | | | FPILTSVFTNEKAKALMDKHLQN |
| 12 | 83 | No putative | ANCCAN_01926 | MKKAVKWGLLAAVVVAVIAAIVAV |
| | | conserved | | VLVLVLKKKDEGPSERVMYFAMH |
| | | domains | | LGDDGALYRNRYEDYKSGMCNL |
| | | | | KMNVDNTRSSIKAMKSLTQKYSFI |
| | | | | LFTDKIETTAPMSSDEAIQKLGSIE |
| | | | | PKAHESPFNQEAVLKEFTGRGGD |
| | | | | NLLVYYIPCSFDYDSEKVKQFVGE |
| | | | | MKKAGLEKKILIVSNTRTEGEIRQL |
| | | | | YEQNNVAGSDTENVAEKIIDFGH |
| | | | | SESEKTTLEPKTSPKPGTTKTTPQ |
| | | | | KPSQEPTKPTRPTKPPGPSPNPN |
| | | | | GLHCLFVGDLYNFGTRTENYDKE |
| | | | | AALFGDIGYDFFEESSISRIGLWA |
| | | | | YGHTNYPKKPDLSNMNSNFEDFY |
| | | | | SDLNNMEFQNTKDPMNTETAIRVI |
| | | | | NGLQDDGSIVNCLVFFSARQDTQ |
| | | | | SLPKLDPQNKKFKRIVAVGYNNT |
| | | | | DLSGVVGSRGVAVSVPYHYLDG |
| | | | | DVATVLDAIMGRPVTKTTPKTVPT |
| | | | | PRPTTSTEVPPMDKSKCLLVGDL |
| | | | | YNFGREDEKYDIEADLISKIGYDFL |
| | | | | LGSDGDLQLALWAYGYTQFSKTV |
| | | | | NDSLKAMRKNYEEFEPDLDKLDF |
| | | | | KDVSNPWSSESAIKAINEMYDSQ |
| | | | | KRVDCLVFFSAQQNPQSLPKLNP |
| | | | | KNTKFRKIIAVGFNNVDLRHIVPA |
| | | | | NGLAFKVPFHYLDEDITPIVKAMH |
| | | | | GRYVPKTTVKPTTKKTTTTAPPPP |
| | | | | KKLNCLFVGDMYNFGSILDHIEGE |
| | | | | VQFMSDLAYDFFGQGENWNAGL |
| | | | | WAYGFTHFPTLPNLKNMKNNYK |
| | | | | DFEKELGTMEFTDRDNYLTTSKAI |
| | | | | EVINRLMDDGSSANCLVFFSAQK |
| | | | | NTNILPRLSPIYKKWERIVAVGFN |
| | | | | DTDLSGVVYGTGVAVRVPFHYLD |
| | | | | EHVNKVMDAIMGRYVPQTTKRPV |
| | | | | TVPTPRPTTPVPPGTGQNCLLVT |
| | | | | DMSNFGRDMDKYEIELDLLSKVG |
| | | | | YDFFEKSNKSVVALWAYGYTDYS |
| | | | | KDVAKSLEKLFPNYRQLATELER |
| | | | | MKLHETSSPLTTAK |
| 13 | 15 | SCP/TAPS | ANCCAN_19762 | QSLIPFGMEMWYCHSVPRSGPQ |
| | | | | LRNVFLDLCPNSAFDKEVIEELVL |
| | | | | GHINDQRYKLLRGLQLNGPWQG |
| | | | | KDKWTPETQGYGKKLPTGKTMN |
| | | | | KLEWSCKLEKNAKAALDPKCTDV |
| | | | | APNAPQGKTGLFYKADIDWEVPD |
| | | | | ITSAAWAWMEQIEKFAVSDDAISD |
| | | | | KQVIFKDHDQHHSKTTIWCIRLGK |
| | | | | VAATKERTVRMDSHAKMECVRK |
| | | | | QTHRRGLHSLMKS |
| 14 | 72 | TTR-52 | ANCCAN_17044 | LRQQSVGITGRLMCGDKPAAGVK |
| | | superfamily | | VKLYDEDDGLDRDDLLDQGFTDA |
| | | | | RGEFTLKGSERETTNIDPVFKVYH |
| | | | | DCDDGIKQSVGITGRLMCGDKPA |
| | | | | AGVKVKLYDEDDGLDRDDLLDQ |
| | | | | GFTDARGEFTLKGSERETTNIDP |
| | | | | VFKVYHDCDDGIKPGQRKVKFHI |
| | | | | PNQYIYSGGVPRRLFNIGVLNLET |
| | | | | IFPDEERDLI |
| 15 | 9 | SCP/TAPS | ANCCAN_06741 | IEFGCKDTLISDKWRETVLNFHNN |
| | | | | NRRKISQGQQVSKAGAVMPAAK |
| | | | | DMNQLYWDCNIEYNAYLLNONNA |
| | | | | QAKLPTDFGENTASINLGRKCKS |
| | | | | PEENTLPILRQWWNEVKLEDLST |
| | | | | PTIDITATPQLKEFAMMANGPTTG |
| | | | | FACTYNKKCSDKLL |
| | | | | CLYNTQPSKNPT |
| 16 | 74 | TIMP | None available | LCPDLDTTLKKSDFDSANIVLGGN |
| | | | | LISVEEKEAEFGLYGRRFNHVVLN |
| | | | | MRYSKFYKWKSEFRKDLSPNKLT |
| | | | | VPEWTYKRCNMTYTTGNVYIVGC |
| | | | | FDLHRCSFMKRLNQFTPNEVSLL |
| | | | | NSL |
| 17 | 11 | SCP/TAPS | ANCCAN_04194 | NEHNLRCPQNGTEMPGFNDSIRL |
| | | | (Ac-NIF) | QFLAMHNGYRSKLALGHISITEES |
| | | | | ESDDDDDFGFLPDFAPRASKMR |
| | | | | YLEYDCEAEKSAYMSARNCSDSS |
| | | | | SPPEGYDENKYIFENSNNISEAAL |
| | | | | KAMISWAKEAFNLNKTKEGEGVL |
| | | | | YRSNHDISNFANLAWDAREKFGC |
| | | | | AVVNCPLGEIDDETNHDGETYAT |
| | | | | TIHVVCHYPKINKTEGQPIYKVGT |
| | | | | PCDDCSEYTKKADNTTSADPVCI |
| | | | | PDDGVCFIGSKADYDSKEFYRFR |
| | | | | EL |
| 18 | 21 | SCP/TAPS | ANCCAN_12561 | MNAITWSCDLEKVTQEGLKSHSC |
| | | | | DSNFPPVDPQGRAQLYTSADNE |
| | | | | YESTVEEFIASRVDLIDSNALKDV |
| | | | | GPNSVKYTDATATSEYSNVMRAT |
| | | | | TTQIACTKKVCTDINMYVIYCITNQ |
| | | | | KALTNGEVIYEVGIAGQCTDCPA |
| | | | | GTVCDPTSKLCVASGGTVSAGTA |
| | | | | STTPATTATAASTTLPTSPEANFP |
| | | | | SGASSKCPNSRMTDTLRDEYVRL |
| | | | | HNFRRGLLAKGQIPRKDGKYLPM |
| | | | | ASNMMAITYDCSLEEGAISWAAQ |
| | | | | CPKASSPQSFRPGLGENCYSFPA |
| | | | | TRFNFDTAAKKSVTEWWKPIRDV |
| | | | | NYFEKVVVFRPFHEGAPISTFTQ |
| | | | | MGWATSNKLGCSIVKCTTSNRYV |
| | | | | GVCRYSPRGNIVNNNVYQVGTP |
| | | | | CNACPSGTTCNSSEGLCF |
| 19 | 17 | SCP/TAPS | ANCCAN_11005 | RPVTFVQPQCQPNGYLDKTTIDN |
| | | | | GILQPINDERAKVAKHEQQNGMS |
| | | | | PTQKLPPATGMTQLKWSCTLEQT |
| | | | | AIKALGETCDYDPPANTGGMADF |
| | | | | FFSYVTLLKFYVIIIFTYLSIMLVFK |
| | | | | PSHFDQGFSRMYEGIQREDFQFF |
| | | | | SFFLGCYTPLKVLLFIIIFVAHQYLP |
| | | | | LFSCSLALFTTSLSFHTNRCIQSL |
| | | | | DSKS |
| 20 | 28 | No putative | ANCCAN_11519 | TAFGCNDRLITDEWRAMILKFHN |
| | | conserved | | NHRRTVAQGNQAMKGGKMPPG |
| | | domains | | NDINELHWDCNIENYASLHNCNN |
| | | | | KIGIMPNFSTISTNIKLTGKPCDINK |
| | | | | ETKTVLNKLWNEAKNADLKDPKY |
| | | | | NDATIKNFGIMAYGNTTGFACTYN |
| | | | | KECSDNLLCWYNRKPENGKPLYT |
| | | | | SGSDCTKGGCTNSTCVDYLCQL |
| | | | | KEYTPTKDFLPRYCHPDVDGLTY |
| | | | | EQHLTAQYMMNYYRRLLATGWT |
| | | | | RDKSAYAPTATALWSVVYLCASI |
| | | | | GKATKALADAVACLDPPFPVARG |
| | | | | YTQTHHVINRLNVDPKEALEEAIK |
| | | | | AWAEQAKKVDLKPIGGAVFYQDE |
| | | | | VEEKASDFAKMITDRNSAFGCGIS |
| | | | | ECKAKSYTLVLCHFNGLFTSRDPI |
| | | | | YTVGKPCSRCAKFNKKCEDALG |
| | | | | GGLCINK |

The inventors have identified the nematode protein families to which the proteins of the invention belong. The nematode protein families are listed in the column “Family” and the associated gene names are listed in the column “Gene name” of Table 2. The inventors have identified that some of the proteins of the invention also show homology to mammalian proteins, including mammalian proteins with anti-inflammatory properties. These include proteins of the invention belonging to hookworm SCP/TAPS, fatty acid- and retinol-binding protein, TTR-52 protein, lysozyme and annexin protein families. Accordingly, in some embodiments of the invention, the protein is selected from a protein family having anti-inflammatory activity, especially SCP/TAPS, fatty acid retinol binding protein, TTR-52 protein, lysozyme and annexin protein families.

SCP/TAPS are cysteine-rich proteins found in all eukaryotes. SCP/TAPS proteins are massively expanded in the genomes of gastrointestinal nematodes and make up about 30% of all excretory/secretory (ES) products in hookworm parasites (Logan J et. al., Comprehensive analysis of the secreted proteome of adult Necator americanus hookworms, PLoS NTD, 2020 14(5): e0008237). While full sequences are often divergent, Cys-spacing and several key resides are typically conserved.

Hookworm Na-ASP-2 is a group 1 SCP protein and contains a tetrad Glu99, His 88, His148 and Glu125 which is present in many hookworm SCPs. Na-ASP-2 has been identified as having a putative fatty acid binding cleft and may bind fatty acids such as prostaglandins and leukotrienes.

Hookworm neutrophil inhibitory factor (NIF) is a group 3 SCP protein. NIF has been shown to be an antagonist of integrin and binds to CD11b/CD18 to block adhesion of activated neutrophils to vascular endothelium. NIF has been shown to be neuroprotective after focal ischemia in rats.

The fatty acid- and retinol-binding protein family (FAR) are α-helix rich proteins specific to nematodes. FAR are involved in the development, reproduction and infection of plant parasitic nematodes and are secreted into plant tissue to disrupt the plant defense reaction.

TTR-52 protein is a transthyretin-like protein which is expressed in and secreted from C. elegans endoderm. The protein clusters around apoptotic cells and mediates recognition of apoptotic cells by the CED-1 phagocyte receptor.

While there is limited literature on nematode lysozymes, human lysozyme is produced by Paneth cells in the cecum and ascending colon. Lysozymes exhibit antibacterial activity and process peptidoglycan in the bacterial cell wall. In healthy subjects, lysozyme is typically abundant in secretions including tears, saliva and mucus and is also present in cytoplasmic granules of macrophages and the polymorphonuclear neutrophils (PMNs), whereas in subjects with an inflammatory bowel disease lysozyme may be abundant in the distal colon where it drives inflammatory antibacterial responses. Large amounts of lysozyme can also be found in egg whites, and hen egg lysozyme has been shown to attenuate dextran sulfate sodium (DSS) colitis in pigs.

Annexin A1 is a glucocorticoid-induced molecule that replicates many of the described anti-inflammatory effects of glucocorticoids. Annexin A1 is abundant in human PMN leukocytes and inhibits protein complex NF-κB, a key regulator of inflammation. Mice deficient in Annexin A1 have been shown to exhibit more severe DSS colitis (de Paula-Silva M et. al., Role of the protein annexin A1 on the efficacy of anti-TNF treatment in a murine model of acute colitis, Biochem Pharmacol, 2016, 115, 104-113) and levels of annexin A1 have been shown to be reduced in RA patients compared to healthy controls. Previous studies have shown that the annexin MC12 tripeptide when cyclized protected against TNBS colitis in mice (Cobos Caceres C et al., An engineered cyclic peptide alleviates symptoms of inflammation in a murine model of inflammatory bowel disease, JBC, 2017, 292, 10288-10294). Annexin A1 mimetic peptides and agonists that target the N-formyl peptide receptor 2 (FPR2) are of interest as therapeutics for RA (Yang Y H et. al., Annexin A1: potential for glucocorticoid sparing in RA, Nature Reviews Rheumatology, 2013, 9, 595-603).

In some embodiments, the amino acid sequence of the protein has at least 70%, at least 80%, at least 90%, at least 95%, at least 98%, or at least 99% sequence identity to any one of SEQ ID NOs:1-20. For example, the amino acid sequence may have at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, at least 90% sequence identity to any one of SEQ ID NOs:1-20, especially any one of SEQ ID NOs: 1, 4, 6, 8 and 11, more especially any one of SEQ ID NOs: 4, 8 and 11.

In some embodiments, the amino acid sequence of the protein is any one of SEQ ID NOs:1-20, that is, the amino acid sequence has 100% sequence identity to any one of SEQ ID NOs:1-20, especially any one of SEQ ID NOs: 1, 4, 6, 8 and 11, more especially any one of SEQ ID NOs: 4, 8 and 11.

In some embodiments, the protein has an amino acid sequence having at least 70% sequence identity to any one of SEQ ID NOs:1-8. In these embodiments, the amino acid sequence of the protein may have at least 70%, at least 80%, at least 90%, at least 95%, at least 98%, or at least 99% sequence identity to any one of SEQ ID NOs:1-8. For example, the amino acid sequence may have at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, at least 90% sequence identity to any one of SEQ ID NOs:1-8, especially any one of SEQ ID NOs: 1, 4, 6 and 8, more especially any one of SEQ ID NOs: 4 and 8.

In some embodiments, the amino acid sequence of the protein is any one of SEQ ID NOs:1-8, that is, the amino acid sequence has 100% sequence identity to any one of SEQ ID NOs:1-8, especially any one of SEQ ID NOs: 1, 4, 6 and 8, more especially any one of SEQ ID NOs: 4 and 8.

In another aspect, the present invention provides a protein comprising an amino acid sequence in which a total of up to about 250 amino acids have been substituted, inserted and/or deleted in any one of SEQ ID NOs:1-20, especially any one of SEQ ID NOs: 1, 4, 6, 8 and 11, more especially any one of SEQ ID NOs: 4, 8 and 11, or a biologically active fragment or variant thereof.

In some embodiments, the amino acid sequence of the protein has a total of up to about 250, up to about 225, up to about 200, up to about 175, up to about 125, up to about 100, up to about 75, up to about 50, up to about 40, up to about 30, up to about 25, up to about 20, up to about 15 up to or about 10 amino acids which have been substituted, inserted and/or deleted in any one of SEQ ID NOs:1-20, especially any one of SEQ ID NOs: 1, 4, 6, 8 and 11, more especially any one of SEQ ID NOs: 4, 8 and 11. For example, the amino acid sequence may have a total of up to about 250, up to about 245, up to about 240, up to about 235, up to about 230, up to about 225, up to about 220, up to about 215, up to about 210, up to about 205, up to about 200, up to about 195, up to about 190, up to about 185, up to about 180, up to about 175, up to about 170, up to about 165, up to about 160, up to about 155, up to about 150, up to about 145, up to about 140, up to about 135, up to about 130, up to about 125, up to about 120, up to about 115, up to about 110, up to about 105, up to about 100, up to about 95, up to about 90, up to about 86, up to about 80, up to about 75, up to about 70, up to about 65, up to about 60, up to about 55, up to about 50, up to about 45, up to about 40, up to about 35, up to about 30, up to about 25, up to about 20, up to about 15, up to about 10, up to about 5, or up to about 3 amino acids which have been substituted, inserted and/or deleted in any one of SEQ ID NOs: 1-20, especially any one of SEQ ID NOs: 1, 4, 6, 8 and 11, more especially any one of SEQ ID NOs: 4, 8 and 11.

In some embodiments, the amino acid sequence has a total of up to about 250 amino acids which have been substituted, inserted and/or deleted in any one of SEQ ID NOs:1-8, especially any one of SEQ ID NOs: 1, 4, 6 and 8, more especially any one of SEQ ID NOs: 4 and 8, or a biologically active fragment or variant thereof. In these embodiments, the amino acid sequence may have a total of up to about 225, up to about 200, up to about 175, up to about 125, up to about 100, up to about 75, up to about 50, up to about 40, up to about 30, up to about 25, up to about 20, up to about 15 or up to about 10 amino acids which have been substituted, inserted and/or deleted in any one of SEQ ID NOs:1-8, especially any one of SEQ ID NOs: 1, 4, 6 and 8, more especially any one of SEQ ID NOs: 4 and 8. For example, the amino acid sequence may have a total of up to about 250, up to about 245, up to about 240, up to about 235, up to about 230, up to about 225, up to about 220, up to about 215, up to about 210, up to about 205, up to about 200, up to about 195, up to about 190, up to about 185, up to about 180, up to about 175, up to about 170, up to about 165, up to about 160, up to about 155, up to about 150, up to about 145, up to about 140, up to about 135, up to about 130, up to about 125, up to about 120, up to about 115, up to about 110, up to about 105, up to about 100, up to about 95, up to about 90, up to about 86, up to about 80, up to about 75, up to about 70, up to about 65, up to about 60, up to about 55, up to about 50, up to about 45, up to about 40, up to about 35, up to about 30, up to about 25, up to about 20, up to about 15, up to about 10, up to about 5, or up to about 3 amino acids which have been substituted, inserted and/or deleted in any one of SEQ ID NOs: 1-8, especially any one of SEQ ID NOs: 1, 4, 6 and 8, more especially any one of SEQ ID NOs: 4 and 8.

Suitably, the protein of the invention or biologically active fragment or variant thereof may be isolated, purified, substantially purified, enriched, synthetic or recombinant.

In one aspect, the present invention provides a recombinant protein encoded by a nucleotide sequence having at least 70% sequence identity with any one of SEQ ID NOs:21-53, especially any one of SEQ ID NOs: 21, 24, 26, 28 and 31, more especially any one of SEQ ID NOs: 24, 28 and 31, even more especially any one of SEQ ID NOs: 24 and 28, or a biologically active fragment or variant thereof.

SEQ ID NOs:21-40 encode the proteins of SEQ ID NOs:1-20, respectively, and are set out in Table 3. SEQ ID NOs:21-40 correspond to native worm genome nucleotide sequences.

TABLE 3

| SEQ | | Nucleotide sequence encoding |
|---|---|---|
| ID | Gene name | recombinant protein |
| 21 | ANCCAN_22177 | CAAGCTCCAGGTTATTATCAGTGCTGGAACTTTGGCCAG |
| | | ACGAATGAGATAAGACATATCTATTTATCCGAAGTTAACA |
| | | AACTAAGACAGCCAATCGGCGAAGGAAAAGCTCCAAAC |
| | | AAGGATGGCATGTGCCCACAGGGGAAGAATATTTATAA |
| | | GCTGTTCTGGGACTGTATTCTGGAAAACTATGCCCAAGA |
| | | AGCAGTGGACAAATGTGGTGCAAAGCCAACAGTGCCGG |
| | | CCGAATTATCGATGGTTTGGAACGAAACAACGCTAACCA |
| | | CATGCAATCCAACTCCATCATTCAAGAAGCAGGTGAATG |
| | | ACTGGTGGGATGAGGTGAAAAGAGTAGGACTGGGCAC |
| | | GGATGCCGCCTTCAAACCGGGCTTGGAGAATTTTGCCG |
| | | TGTTGGCAAATGGATTGGCAACAAGGATTGGCTGCGCA |
| | | CAAAAGAATTGCAACGGAAAACTCCAGATGGCATGCGT |
| | | GGTTTACGGCAATAGCAACAACCATGGCTCTCATTTTTA |
| | | TCCTACCACTATCCTGATGCTGTGTTCAAAGAAGTTCTT |
| | | GGATAACATCTGCCCACACTCTCCACCAGACGCGAACA |
| | | AAGATCTAGCTAGAGAAGAACTCTTGAAAGTGCACAATG |
| | | AAAAGAGAGCAGCAATCGCTAAAGGAGAAGTGAAAATG |
| | | GGAAATGGAAAGATGGCTCGTCGGTGTCCACGAATGAA |
| | | GAAATTTGTTAGATATAATTGCACTCTTGAAGTAGCAGC |
| | | CTATGAAAAAGCTAAAACGTGCCCTACAGCAGATCCGAA |
| | | TATTGAGAATGTTAGCTGTTTCCTGTCGAATGCAAGAAA |
| | | TAAGAAGCTAGCCGCGCGCGAGGCCATGGATGCCTGG |
| | | TTTGGAGAAATTAGTAAAAGCAATGCCCATATGGAGCAA |
| | | GCGACAGGCAGCCAAAATCTGCTTCTACCAGCTCTCAA |
| | | CATAAGGCAGTTTGCCAGGATGGTTTGGGACCGCAATG |
| | | CTGAGATGGGCTGCGGAATCGCAGAGTGCAAAGGCAG |
| | | CCATAAATATGTCGTGGTTTGCCAATATGGACAGGGTGT |
| | | GGGCAAATATGGAGATACCATCTATATGATGGGTCCAAC |
| | | CTGTAACCAATGCGGCGGCCCAAGTGGCGGCAGATGTA |
| | | CAGACGGCGCATTCTGCCCCTAG |
| 22 | ANCCAN_12564 | CAAAATGCATACTGTGACGGTGGAGACCTGCTCGGAGC |
| | | TGAAATCTTCGAATACATTCTCGATCCAGTGAACGAACA |
| | | CCGAACGACTTTGGTTGATGGAACTCAAAAGAATGGTCT |
| | | CTCCGGCCAAAACTTGCCGCCAGCAAAAGGCATGACTC |
| | | GACTGGATTGGGACTGTGATCTTGAAAAGGAAGCTAAAA |
| | | AGATTGTAACCAGTCATTGTTTCGACTACGTTGATGAAC |
| | | CTCCCGTTATGAATAACAGGGCTCAAGTATTTCTCTCAG |
| | | CATACGCGGACGGTCTGCCTAATAGTCCTGTGGTAATG |
| | | CAACAACTCTTCGCACAAGCTCTAGCTGAAATTGATACT |
| | | ACTGCCCTCGAAGGTGTCACCCCCGGCGCGACTAACGT |
| | | CATCTACAAATCTGGCAGCGTTGAAAATCTGTTGAACTA |
| | | CTTCAACCTAATGCGACCCGCGAATACACGTATGGGAT |
| | | GTGCTTGGACGAAGTGTACTAATGTCGATGGAGACGAG |
| | | TTCATCAATGTGTACTGCCTTTTGAACTCACAACAACTCA |
| | | AAGACGGCGATGTCATCTACAATGTGGGAACAGCTGGA |
| | | GTTTGCTCCGACTGCCCTACTGGGACAAGTTGTGATCG |
| | | AAATACGAAACTATGTGTTCGTACTACTTCTGTACCAAC |
| | | CACCGGTCCCGCGGCAACGACCACACCATCAACAACCA |
| | | CAACTACACCTGCACCCGCTCAAACAACAACAACACCTC |
| | | CTACTTCTCCGGAAGCGAATTTCCCTTCAGGTGACAGCA |
| | | GTCGATGTTCTCATCCCCTTGCAAGCAGGATGACTAATA |
| | | CTCTCCGGAGTGAATACGAGAGATTACACAATTTCCGAA |
| | | GAGGCCTTCTCGCAACCGGACAAATTCCAAGAAAGGAC |
| | | GGGAAACATCTACCAACAGCTTCTAATATGCAGAAGATG |
| | | ACATATGACTGCACCTTAGAGGAGGGAGCCATACAATAT |
| | | GCTAGCACGTGCCCCACGAGACTTTCCGAATTGAGTTC |
| | | CAGACCAAATGTGGGAGAGAACTTCAAAACATTCCCAG |
| | | CGACGAGGTTCACATTTGACACAGCAGCTAAAAAATCCG |
| | | TTACTGAGTGGTGGAAGGTGATTCGTGACGTGAACTTCT |
| | | TCGAGAAGGTCGTCGTCTTCCGCCCGTTCCATGACGGA |
| | | CATCCGATATCCTCCTTCACACAGATGGCATGGGCCAC |
| | | AAGTAACAGGCTGGGATGCTCCATCGTCAACTGCTCGA |
| | | CATCCAACCGATACGTGGGCGTATGCCGATATACGCAA |
| | | AGGGGAAATATCGTAAATAACAACGTCTACGAAGTTGGA |
| | | GCCGTTTGCAGTGCCTGTCCCAGTGGAATGACGTCCTG |
| | | CAACAGGGATGAAGGACTGTGCTTCTAA |
| 23 | ANCCAN_12569 | ATGTCATTTACAACGTGGCTACAACAACCGCACCGGTG |
| | | CGCTGGCAGTGCCACTGCTGACAGGATGACTGATACTC |
| | | TTCGCGATGAGTTCGAGAGATTGCACAATTTCCGAAGAG |
| | | GCCTGCTCGCAACCGGACAAATTCCACGAAAGGACGGG |
| | | AAATATCTACCAAAAGCTTCTAATATGCGGAAAATTGTAG |
| | | ATTTATTTTCACTTAACCCATTTGCGGCGATCTATTATGC |
| | | AACAATTCAGATATATGATTGCGCCTTAGAGGAAGGAGC |
| | | CATACAATACGCGAGCCAATGTCCCACGACACATTCCG |
| | | AACAGAGCTCGAGACCGAATGTGGGGGAGAACTTCAAA |
| 24 | ANCCAN_07727 | ACATTCCCAGCGACAAGATTCACATTCGACACAGCAGCT |
| | | AAGAAGTCTGTTACTGAATGGTGGAAGCCGATTCGTGA |
| | | CGTGAACTACTTCGAAAAGGTCGTCGTCTTCCGTCCCTT |
| | | CCATGACGGAGCACCGATATCCTCTTTCACACAGATGG |
| | | GATGGGCCTTAAGCCACCAGGTGGGGTGCTCCATCGTC |
| | | ATGTGCTCGGCGTCCAACCGCTACGTAGGCGTGTGCCG |
| | | ATATTCGCCAAGAGGCAACATCGTGGATAATAACGTCTA |
| | | CATGGTAGGAAACCCCTGTAGTGCCTGCCCCAGCGGAG |
| | | CGACCACCTGCAGCAGCGCCGAAGGGCTGTGCTTTTAG |
| | | ATGAACTCTCCATACGCTACGATCGTCGACAACCAGGAT |
| | | TTCCATCCTGCCCTGTACGCAGAGAAAATCGACCGTGC |
| | | TCTGCGAGCAGGCGACAAGGACACCATCGTGAAGGTGC |
| | | TCACCTCGATCAGCAACAACCAGCGACAGCAGCTGCGT |
| | | GAGCCATACAAAATCAAATATGGCAAGGACATCATCGCT |
| | | GCGCTCGACAAGAAGTTCAGTGGCGATCTCGAGAGGAC |
| | | CATCTTCGCTCTTATGGATACTCCACTTGACTACGACGT |
| | | CAAGCAGCTGAAGCAGGCTATGAAGGGCCTTGGCACTG |
| | | ACGAGGCTGTGCTGATCGAAATTCTCTGCTCCCGAACT |
| | | CCCGACCAGATGGCTGCTATCCGAATTGCCTACGAAAG |
| | | AGAGTACAAAACGCCACTGGAGAAAGACGTTGGTTCTA |
| | | CATTCCATTCAGTTCGAGAGAAAATGAAAGCATTGAAAA |
| | | CATTAGATATGGTAGCGTCAGACACATCCGGAGAGTTCA |
| | | AGGATCTGCTTGTCGCACTTGTCACCGGTAGCAGGGAC |
| | | GGCAGCCGCAACACCAACGATCAGGAGGCCAAAGATGA |
| | | CGCTGTCCGTCTCTACGCGGACGGCAAGGCGAAATTGG |
| | | TCGGCAAAGACTCGCCATCCCACTTCCTCAAGATTCTTG |
| | | CCACCCAAAATCAGTACCAGCTTAGGAAGGTAAGTGTTT |
| | | CTCATTGCATGGTTAATCATCGCGAAGTAAATGTTGGTG |
| | | TTAAGGTGTTCGCTGCTTTCGCGGATCTTTCTGGATCCA |
| | | CCATCGAGAAGGCGATCGAGAAGGAGTTCAGCGGTGAT |
| | | CTCCAGAAGAGTTACCTCACCATCGTCCAAGCCGCTTC |
| | | CGACAAGCAGAAGTTCTTCGCTAGGCAGCTGTACAACT |
| | | CGATGAAGGTAGGCCTGGGAACCCGCGACAACGACCT |
| | | CATCCGTGTGCTGGTTACCCGCTCGGAGGTGGATCTTG |
| | | AGCTCATCAAGCAGGAGTTCCAAGCGCTTTACAACAAG |
| | | CCACTCGAAGACATGATCAAGGGAGACACCTCTGGTCA |
| | | GTACCGAGACGCTCTTCTCGCCATCGTGGTCGGAAACC |
| | | GCATGTAG |
| 25 | ANCCAN_00478 | AATTCTATGAGATGCGGAAATAATGGAATGACCGACGAA |
| | (Ac-ASP-2) | GCCCGGCAGAAATTCCTCGACGTGCACAACAGTTACAG |
| | | ATCTATGGTTGCCAAAGGACAGGCAAAGGATGCAATTTC |
| | | GGGAAATGCTCCGAAGGCTGCCAAAATGAAGAAAATGA |
| | | TCTACGACTGCAACGTCGAATCAACTGCAATGCAAAATG |
| | | CGAAAAAATGTGTTTTCGCCCATTCGCACAGGAAGGGA |
| | | GTTGGCGAAAATATTTGGATGTCGACTGCGCGTCAGAT |
| | | GGACAAAGCACAAGCTGCTCAACAGGCTAGTGACGGTT |
| | | GGTTCAGTGAGCTTGCGAAGTATGGTGTAGGCCAGGAA |
| | | AACAAGCTAACAACGCAGTTGTGGAACAGGGGAGTTAT |
| | | GATAGGACATTACACTCAGATGGTCTGGCAGGAGTCCT |
| | | ACAAACTCGGATGTTATGTGGAATGGTGTTCATCGATGA |
| | | CCTATGGTGTCTGCCAGTACAGTCCTCAGGGTAATATGA |
| | | TGAACTCACTCATCTACGAGAAAGGAAACCCGTGCACAA |
| | | AAGACTCTGACTGTGGCTCGAACGCCAGTTGCAGCGCT |
| | | GGGGAGGCGCTTTGCGTCGTGCGTGGCTAG |
| 26 | ANCCAN_07322 | AAAAGCAATCGCTCCTGAAGGAGTAGTGAAAGCAGCCG |
| | | CTGGCTATGCTTACGCCGTCGATCTCGAAGTGCCAGTT |
| | | CCACTGAGCGGTTTCAACTGCATGAAAAAATATGGCTAC |
| | | AAAACTGTATTCATAAGGGCATATGATCCCACAGGTGCT |
| | | GGCCGGTTCGATACAAATGCTGTTAACAACATTOGCAAC |
| | | GCTAACCAAGCTGGCCTGGGAACCGAGGCGTACATGAC |
| | | ACCGCAGCTTCACTCGAACAAGAGAGGCAGTCAGCAAT |
| | | TTCGAGAACTCTATGAGGGCCTCAGGAACGCCAAAATC |
| | | CAAGTGAGGACCGTCTGGGTGCAGGTCACCTCACCCGT |
| | | TAACTGGGGACCCAACACTCAAGCAAATATCTATTTCAT |
| | | CAACGACATAGTATCCATGGCAAAGTACTATGGTGTTAC |
| | | GATTGGATTCTATACAAATGTCTACGACTGGAACCAGAT |
| | | CACAAAAGGCGCCAACGTAGAGGGAGCTATGCTCTGGT |
| | | ACTGGAACGTGAACGGAGCCGGAGTGAACGGTGAGAC |
| | | GCCGGCCAACTTCGACGACTTCCGCCCATTCGCGAAAT |
| | | TCACGAAACCGACCGTGAAGCAGTTCGGACAGGTGGAA |
| | | CATCTGTGTGGAGTCACTGTGAACAGGGACGTCTACAC |
| | | GCTGAACAACTCCATAAACCTCGTCTCCTCAAAAGCAGA |
| | | GAAATTGGACGGAGAGCTGATTGTTGGCACCCTTGGAG |
| | | GTAATCAGATGTCGGGTCTTGTACAAAGTGTGTAA |
| 27 | ANCCAN_26187 | GCTGTGGTTCCCGATGTACTCGTCACTGTCGAAGCAAC |
| | | GACAAGATTCAACTGCAGAAACTCGCTGATTTCTGACGA |
| | | GTGGAGGCAATATGTTTTAGACGAACTTAATGACTATCG |
| | | GAGATCTCTCGCACGTGGTGAAGTATACGACGCGACGA |
| | | AAAATTTCTTGCCCAAGGCTAAAGACATGAACGAATTGA |
| | | AATGGGACTGCAATTTGGAAGAGGTGGCATATAAGAGC |
| | | TTGCAATCGTTAATGAGTACTTGCGATGTACCTCTTTTCA |
| | | GTATCTCTCGCCCATATCAAACCATGCAAAAACGGTTCA |
| | | AGAGGAAGGAATGCAACATCACCAGCCAAGCTAAAGTA |
| | | TTGCTATACGGATGGTGGAAGGAAATTAAGAACACAACT |
| | | GTGAAGTATGTGAATGGCAACCCCAGTGTTCCGACTGG |
| | | CTTGGAATTTATCGTGATGGCCACCGCCACAACGACAG |
| | | GATTTGCCTGCACATATGAGTTTTGTAATAATATGCCGA |
| | | GAGGGATTATTGCCTGCATTTACGACGATAAAACAACAG |
| | | ACGTGTCTGTTGCTAACAGAATTATCTACCAAGCAACGC |
| | | AGAATACGCAAGAAATTTGTAGCTCGTGCAGCAAGAATT |
| | | GCATCAAATCCCTCTGCAATGTTCCACATACTCCTGTTA |
| | | CTCTTCCTACGACCTGCTCAGATGATAAGTTGACGCAAG |
| | | ACTCGAATAATGCGGCACTATGGATGCACAACTATTATA |
| | | GAAAGCTACTAGCATCGGGATGGGCCAAGGATAAGAAG |
| | | TCCAAGAGTGGTTATGCTTTAACTGCGAAAAAGATGCTA |
| | | AAACTAGAGTACGACTGTGCTCCCGCCGCTGGCGGAAT |
| | | TTCAAACGGTGCCACAAAGACTTATGATTTAATCAAGGA |
| | | CTGTCCGAAAAGTGGGAATCCCACACCGTCGAACGGGT |
| | | ACTCCATGAATTTTTTGAGGCTTAAAAACCACACGATATC |
| | | GGAACAAGAGGCTCTTGAGCAGGCGATTAAGAAATGGT |
| | | GGGGAGTTCTCGAAACGAAGGGCCTTGGCTCCGAAAAC |
| | | ACGTTCAGCGACAACTCTGAAATTGCCAGTTTTGCCAAA |
| | | ATGGCACACGACGGAACCGAAAAGCTTGCATGTGCGGT |
| | | TCAGAACTGTAAGCAGAATGGAGAAACGCTTGTCGCGT |
| | | GCCAGTATTCTCCAGCGATCCAAGATGGCGACAAGATTT |
| | | ACGAGACAGGAAAAGTATGCGGTGGTTGTAAAGCTCTC |
| | | AGCAAGAAATGTTCGGACCCAGCGGGACTTTGTGAGTA |
| | | A |
| 28 | ANCCAN_08034 | AATGCAGAACGTCACCGTCAAGGGGACCACCATCTGCA |
| | | ACAAGAAGCGAATGGCCGATGTGACGGTGGAACTGTGG |
| | | GAGAGAGACACCCTCGACCCCAACGACCTCCTCGACTC |
| | | CAAGAAGACCTCTAGGGAAGGCGAGTTCCTCGTGAAAG |
| | | GTGGTCAGAACGAAGTCGGCTCGATTGAGCCATTCCTC |
| | | AGAATTACACACACCTGCAATGTCAAGAAACCGGGCTG |
| | | CAAGAGAATCACTGAGTTCGACATCCCGAAGTCGAAGA |
| | | TCGACACGGTCTACGACATGACCTACGTGACGCTGGAT |
| | | ATCATTTCCGCAGTCGATAAGGAGAAGTGCTAG |
| 29 | ANCCAN_07062 | CCAAATTACCAGTGCTGGAACTTTGGCCAGCAGGATGA |
| | | GATAAGACGAGACTATCTAGTCGAGGTTAACAAACTAAG |
| | | AGAGACAATCGCCACCGGAACAGCACCCAGCAAAGATG |
| | | GCCGCAAGTGTCCTCAAGGAAAGAACATTTATAAGCTGA |
| | | ATTGGGACTGTATTCTGGAAAACTATGCGCAAAAGGCTG |
| | | CGGACGAATGCGCTGAACCGAAGACGGTTCCAAAAAAA |
| | | TTGTCATTTGTTTACAATGCAACAACGCTAACCACGTGC |
| | | AATCCAATCCCTGATATCAAGAAACAGGTGAACAATTGG |
| | | TGGAGTGAGGTAGAAAGAGTAGGACTGGCCAAAAATGC |
| | | CGCTTTCAAACCCAAGTTAAGGAATTTCGCCGTGTTGGC |
| | | AAATGGATTGGCAACAAGGATTGGCTGTGCACAAAAGA |
| | | ACTGCAATGGAGTACTGCACATGGTGTGTGTGGTTTATG |
| | | GCAAAGCAGCTAGCAGCACTAGGCAAGCCATATACGAA |
| | | GTGGGAGCAGGCTGTGAAGGAAACTACCAGTGTACAAC |
| | | TTACAAAGAATCGAAATGTCTAACAAAGAGAAAATTATGT |
| | | GAGGCCGGCTACCCAACAAGTGAAGCGGTAGTTCCACC |
| | | AACCGTGAGCACTTCTAAACCAACGATAACATCGACAGA |
| | | AGCAACACAACCAGAACCACAACCACCAAAACCAGAGC |
| | | CACCAAAACCAGAACCACCAAAACCAGAAACAACAAAG |
| | | CCTCCTGTAACATCTGAGCACTGCCCAAAGCAAGAACA |
| | | CATGAACACTGATCTAGCTAGAGAAGAATTTGTGAATCT |
| | | GCACAATCAAGCTAGGGCAACAATTGCTAAAGGAGGAG |
| | | TCAAAATGGGAGATGGAAAGATGTCTCGCCCGTGTCCG |
| | | CGAATGAAGAAATTTGATATAAAGAATTATGATTGCAAAC |
| | | TGGAGGAAGTGGCGTACGCAACAGCCGCTAAGTGCCAA |
| | | ACAGAAGTTTACGTTCAGAATGAAAACTGGTTTATGGCG |
| | | TCGACGAAAGCAACAAATAGAAAGCTAGCAGCTAGAGA |
| | | GGCCGTGGAACACTGGTACAATGAAATTAAATCAGGCC |
| | | ATATGGAGCAAAAAACAGGGAGTCAAAATTTGCTCATGC |
| | | CCAGCTTCAAAATAGACCACTTTGCAAGGATGGTTTGGG |
| | | ACACCAATACTCAAATCGGCTGCGCAGCCCACAAGTGC |
| | | GGTAAAATTTACCACGTAGTGTGCCGCTATGGACCGAA |
| | | GGTTGGCAAATATGGAGATACGATTTATATGATGGGTCC |
| | | AACCTGCAACCAGTGCCAAGGTAGATGCATAGAGGGAG |
| | | CTTTTTGCCCTTAG |
| 30 | ANCCAN_13497 | TGCAAGTGCGAAAAGAAACCTCGTCCTCCATTGGAGAA |
| | (Ac-TIMP-1) | ACTGCTTTGCCAATCACAATTTGTTACTCACGCGAAAGT |
| | | GACGAAGAAGAGAATTGATGGTTACTTCATCTATTACGA |
| | | CTTGGAGCATAAGGAAGTTTATAAGCCCAAAGATAGGAG |
| | | TATCCCAATCGAACTCTTCTCATGGAGGGAAAAGGAAAA |
| | | TTGTGGTGTTCCGGATCTCGAAGAAGGCAAAGAATACCT |
| | | GATAGGAGGTAAAGTGACGGATTATGGCGACGGTGATT |
| | | TGGTAATTTCTGTTTCACGGTGCGACCTTCTCCGAAACT |
| | | GGACAGACGTCTCTGGAGAGGAGAAGAAATTGCTCGGA |
| | | ACGTTCAAATGTGAAAATCAGTCA |
| 31 | ANCCAN_10127 | GCCCCTAACGTTGAGGTGAACAAGTTTGAGGATATTCCT |
| | (Ac-FAR-2) | GAGCAGTACAGAGAACTAATCCCCAAAGAAGTTGCCGA |
| | | CCACCTCAAAGCCATCACCGATGACGAGAAGAAGATCT |
| | | TGAAGGAGGTGCTGAAGGACTACGCCAAATACAAGGAT |
| | | GAAAACGAGTACTTGGCAGTCCTGAAGGAAAAGTCTCC |
| | | CAGCCTACACGAGAAGGCCAAGAAGTTCCACGAGTTTA |
| | | TTAAGGCTAAGGTCGACGCACTTGGGGATGAACCGAAG |
| | | GCGTTCGTGAAGAAAGTTATCGCTGCCGCTCGAAAACT |
| | | GCATGCAGAGCTCCTTGCCGGAAACAAGCCTTCTCTTG |
| | | AGGAACTGAAGAACACTGTCAAGACGTACATGGCCGAA |
| | | TTCAACGCCTTGTCACCCGCTGCCAAAGAGGATCTGAA |
| | | GAAAAACTTCCCTATTCTCACCTCAGTTTTTACGAACGA |
| | | GAAGGCAAAGGCGTTGATGGACAAGCACTTGCAGAACT |
| | | AG |
| 32 | ANCCAN_01926 | ATGAAGAAGGCAGTCAAATGGGGACTTTTGGCTGCTGT |
| | | TGTAGTGGCGGTTATCGCCGCTATAGTCGCGGTTGTCC |
| | | TCGTGCTCGTGTTGAAAAAGAAGGACGAGGGACCTTCC |
| | | GAACGAGTTATGTATTTTGCAATGCACCTCGGAGATGAT |
| | | GGTGCGCTGTATAGGAATAGATATGAGGATTATAAAAGT |
| | | GGCATGTGCAACCTTAAAATGAATGTGGATAACACCCGC |
| | | AGTTCGATAAAGGCTATGAAGAGTTTGACACAAAAGTAC |
| | | AGCTTCATATTGTTCACGGACAAAATAGAAACGACGGCA |
| | | CCAATGAGCAGCGACGAGGCGATCCAGAAATTGGGCTC |
| | | CATTGAACCGAAGGCGCATGAATCACCGTTCAACCAGG |
| | | AAGCGGTTCTGAAAGAATTCACTGGCCGAGGTGGAGAC |
| | | AATCTTCTTGTATACTACATCCCATGCAGCTTCGATTATG |
| | | ACTCCGAAAAGGTGAAACAGTTCGTTGGAGAGATGAAG |
| | | AAGGCAGGATTAGAAAAAAAGATACTTATCGTTTCGAAT |
| | | ACACGAACAGAGGGCGAAATCAGGCAGTTGTACGAGCA |
| | | AAACAACGTCGCTGGATCCGACACAGAGAACGTCGCGG |
| | | AGAAAATCATCGATTTCGGTCACAGTGAATCCGAAAAGA |
| | | CAACGCTGGAACCAAAGACGTCGCCAAAGCCTGGAACC |
| | | ACTAAGACCACACCGCAAAAGCCTTCGCAGGAGCCCAC |
| | | GAAGCCGACAAGACCCACCAAGCCTCCAGGCCCTTCAC |
| | | CAAACCCTAACGGACTGCACTGCCTCTTCGTTGGTGATC |
| | | TGTACAATTTCGGAACTAGAACAGAAAACTACGATAAGG |
| | | AAGCGGCACTATTTGGTGACATCGGTTACGATTTCTTCG |
| | | AAGAGTCGTCCATTTCGAGGATCGGTCTGTGGGCCTAT |
| | | GGACACACGAACTACCCCAAGAAACCAGATCTTAGCAAT |
| | | ATGAACAGCAATTTCGAAGACTTCTATAGTGACTTGAAT |
| | | AATATGGAGTTCCAGAACACCAAAGATCCTATGAATACT |
| | | GAAACCGCAATTAGAGTGATCAACGGTCTACAAGACGAT |
| | | GGCAGTATCGTCAATTGCCTCGTTTTCTTCTCAGCACGG |
| | | CAAGATACCCAGTCCCTTCCAAAGCTCGACCCACAGAA |
| | | CAAGAAGTTCAAGAGGATTGTCGCAGTTGGATACAACAA |
| | | CACGGATTTGAGTGGAGTGGTAGGATCAAGAGGAGTAG |
| | | CCGTTAGTGTCCCTTATCACTACTTAGATGGTGATGTCG |
| | | CAACGGTCCTCGATGCAATCATGGGAAGGCCGGTAACG |
| | | AAAACGACACCTAAGACGGTTCCGACCCCTAGACCTAC |
| | | TACTTCTACAGAAGTGCCACCGATGGACAAATCAAAATG |
| | | TTTGCTGGTTGGAGATCTGTACAACTTCGGTCGCGAGG |
| | | ACGAGAAATATGACATTGAAGCGGATCTTATTAGCAAGA |
| | | TCGGTTACGACTTCCTCCTTGGTTCCGACGGCGATCTCC |
| | | AGCTTGCGCTTTGGGCATATGGCTACACCCAATTCTCTA |
| | | AAACAGTGAACGACTCACTCAAAGCGATGCGTAAAAACT |
| | | ATGAGGAATTCGAACCTGATCTGGACAAATTGGACTTTA |
| | | AAGATGTTTCCAATCCATGGTCATCGGAGAGCGCCATAA |
| | | AGGCTATCAATGAGATGTACGACAGCCAGAAACGAGTA |
| | | GACTGTTTGGTTTTCTTCTCAGCTCAGCAGAATCCTCAG |
| | | AGCTTGCCGAAGCTAAATCCGAAGAACACGAAGTTCAG |
| | | GAAGATCATTGCAGTGGGATTCAATAATGTGGACCTGC |
| | | GACACATTGTACCTGCTAACGGGTTAGCCTTCAAAGTTC |
| | | CTTTCCATTACTTGGATGAAGATATCACCCCAATTGTGA |
| | | AGGCAATGCACGGCAGGTACGTGCCGAAAACGACGGT |
| | | GAAACCTACCACCAAGAAAACGACAACAACTGCTCCAC |
| | | CTCCTCCAAAAAAGCTTAACTGCCTATTTGTCGGAGATA |
| | | TGTACAACTTTGGAAGCATTCTGGATCACATCGAAGGGG |
| | | AAGTACAATTCATGTCCGATCTTGCGTACGATTTCTTCG |
| | | GCCAGGGTGAGAACTGGAATGCTGGTCTTTGGGCGTAC |
| | | GGATTCACACATTTCCCCACACTTCCAAACCTCAAGAAT |
| | | ATGAAAAACAACTACAAAGACTTTGAAAAAGAACTAGGA |
| | | ACAATGGAATTCACCGACCGCGACAATTACCTAACAACT |
| | | TCGAAAGCTATTGAGGTCATCAACAGGCTAATGGATGAC |
| | | GGCAGTTCAGCAAATTGTCTGGTCTTCTTCTCTGCACAA |
| | | AAGAACACCAATATTCTCCCTCGTCTCTCGCCAATTTAC |
| | | AAGAAATGGGAAAGAATTGTTGCAGTAGGGTTCAACGA |
| | | CACAGACCTTAGTGGAGTGGTGTATGGAACAGGAGTTG |
| | | CTGTCAGGGTGCCTTTCCACTATTTGGATGAGCATGTGA |
| | | ACAAAGTGATGGATGCAATTATGGGAAGATATGTTCCGC |
| | | AGACCACTAAGAGACCTGTCACTGTTCCTACACCACGAC |
| | | CTACAACGCCAGTGCCGCCAGGGACAGGACAGAATTGC |
| | | CTATTGGTTACTGATATGTCTAACTTCGGCCGCGATATG |
| | | GATAAATACGAAATAGAACTAGACCTCTTGTCCAAAGTT |
| | | GGATACGACTTCTTCGAAAAATCTAACAAATCAGTTGTG |
| | | GCACTTTGGGCCTATGGATACACGGACTACTCTAAAGAC |
| | | GTGGCGAAATCACTTGAAAAGCTGTTTCCAAACTATAGA |
| | | CAGCTTGCAACAGAACTGGAGAGAATGAAGTTGCACGA |
| | | GACCAGCTCACCTCTGACAACGGCGAAGTAA |
| 33 | ANCCAN_19762 | CAGAGTTTAATTCCCTTTGGAATGGAAATGTGGTATTGC |
| | | CACAGCGTACCTCGTAGTGGACCACAACTGAGAAACGT |
| | | CTTTTTAGATCTTTGCCCGAATAGCGCCTTTGACAAAGA |
| | | AGTGATCGAAGAATTGGTTCTTGGCCACATCAACGACCA |
| | | ACGTTATAAACTGCTGAGAGGTCTACAGCTAAATGGACC |
| | | GTGGCAGGGTAAAGACAAATGGACCCCGGAAACGCAG |
| | | GGATATGGAAAGAAGCTGCCAACGGGCAAGACAATGAA |
| | | CAAATTGGAGTGGAGCTGTAAACTTGAAAAGAACGCGA |
| | | AAGCTGCACTGGATCCCAAGTGTACAGACGTTGCACCT |
| | | AACGCTCCTCAAGGAAAGACTGGATTGTTTTACAAGGCA |
| | | GACATTGACTGGGAAGTGCCCGACATAACATCGGCAGC |
| | | GTGGGCATGGATGGAACAAATCGAAAAGTTTGCTGTAA |
| | | GCGACGACGCCATCTCAGATAAACAGGTTATCTTTAAAG |
| | | ACCATGACCAGCACCACTCAAAGACAACGATATGGTGTA |
| | | TAAGGCTGGGAAAGGTGGCTGCGACAAAGGAGAGAACT |
| | | GTCCGAATGGATTCACATGCGAAAATGGAATGTGTGCG |
| | | AAAGCAAACCCATAGGCGAGGCCTTCACTCGCTTATGA |
| | | AATCTTGA |
| 34 | ANCCAN_17044 | CTACGCCAGCAGTCAGTCGGCATTACTGGACGGCTCAT |
| | | GTGTGGAGACAAACCGGCAGCTGGGGTTAAAGTAAAGC |
| | | TATATGATGAAGACGACGGGCTAGATCGTGACGATCTTC |
| | | TTGACCAAGGATTCACAGATGCTAGAGGGGAATTCACCT |
| | | TGAAGGGTTCTGAGCGCGAAACTACTAACATCGATCCA |
| | | GTATTCAAGGTATACCATGATTGTGACGATGGCATCAAG |
| | | CAGTCAGTCGGCATTACTGGACGGCTCATGTGTGGAGA |
| | | CAAACCGGCAGCTGGGGTTAAAGTAAAGCTATATGATG |
| | | AAGACGACGGGCTAGATCGTGACGATCTTCTTGACCAA |
| | | GGATTCACAGATGCTAGAGGGGAATTCACCTTGAAGGG |
| | | TTCTGAGCGCGAAACTACTAACATCGATCCAGTATTCAA |
| | | GGTATACCATGATTGTGACGATGGCATCAAGCCTGGCC |
| | | AACGCAAAGTGAAGTTCCATATCCCCAACCAATACATTT |
| | | ATTCCGGAGGAGTGCCCAGGCGACTATTCAATATTGGC |
| | | GTGCTTAACTTGGAAACTATTTTCCCCGATGAAGAGCGA |
| | | GACTTGATCTGA |
| 35 | ANCCAN_06741 | ATAGAGTTCGGTTGCAAAGACACGCTGATATCTGACAAA |
| | | TGGCGCGAAACCGTGCTGAACTTTCACAACAACAATAG |
| | | GAGAAAAATTTCTCAAGGGCAACAGGTATCGAAAGCTG |
| | | GGGCAGTGATGCCTGCCGCGAAGGACATGAACCAACTA |
| | | TATTGGGACTGCAACATTGAATACAACGCCTATCTGCTC |
| | | AACTGCAACAATGCACAGGCAAAGTTACCAACAGATTTC |
| | | GGAGAAAATACCGCGAGTATCAATCTTGGTCGCAAATG |
| | | CAAAAGTCCTGAAGAAAACACATTGCCAATCCTGCGACA |
| | | GTGGTGGAACGAAGTCAAGCTCGAGGATCTGTCAACCC |
| | | CTACCATCGACATAACTGCAACACCGCAATTAAAGGAGT |
| | | TTGCAATGATGGCTAATGGGCCGACCACTGGCTTCGCA |
| | | TGCACATACAACAAAAAGTGCTCTGACAAACTGCTTTGT |
| | | CTGTACAATACACAGCCCTCCAAAAATCCCACT |
| 36 | None | TTATGTCCCGATCTGGACACCACATTAAAGAAATCCGAC |
| | available | TTTGATAGTGCGAACATCGTTTTGGGAGGTAATCTGATC |
| | | TCGGTGGAGGAGAAAGAAGCAGAGTTCGGTCTGTATGG |
| | | TCGGCGTTTCAATCATGTGGTACTCAACATGCGCTACTC |
| | | TAAGTTTTACAAGTGGAAGTCAGAGTTCCGCAAAGATCT |
| | | CTCCCCTAACAAACTCACCGTTCCGGAATGGACCTATAA |
| | | ACGCTGTAATATGACGTACACAACTGGAAATGTCTACAT |
| | | TGTGGGGTGCTTTGATCTACACAGATGCTCCTTCATGAA |
| | | GCGCCTTAATCAGTTCACACCGAATGAAGTTAGTCTACT |
| | | GAATAGCTTATAG |
| 37 | ANCCAN_04194 | AATGAACACAACCTGAGGTGCCCGCAGAATGGAACAGA |
| | (Ac-NIF) | AATGCCCGGTTTCAACGACTCGATTAGGCTTCAATTTTT |
| | | AGCAATGCACAATGGTTACAGATCAAAACTTGCGCTAGG |
| | | TCACATCAGCATAACTGAAGAATCCGAAAGTGACGATGA |
| | | TGACGATTTCGGTTTTTTACCCGATTTCGCTCCAAGGGC |
| | | ATCGAAAATGAGATATCTGGAATATGACTGTGAAGCTGA |
| | | AAAAAGCGCCTACATGTCGGCTAGAAATTGCTCGGACA |
| | | GTTCTTCTCCACCAGAGGGCTACGATGAAAACAAGTATA |
| | | TTTTCGAAAACTCAAACAATATCAGTGAAGCTGCTCTGA |
| | | AGGCCATGATCTCGTGGGCAAAAGAGGCTTTCAACCTA |
| | | AATAAAACAAAAGAAGGAGAAGGAGTTCTGTACCGGTC |
| | | GAACCACGACATATCAAACTTCGCTAATCTGGCTTGGGA |
| | | CGCGCGTGAAAAGTTTGGTTGCGCAGTTGTTAACTGCC |
| | | CTTTGGGAGAAATCGATGATGAAACCAACCATGATGGA |
| | | GAAACCTATGCAACAACCATCCATGTAGTCTGCCACTAC |
| | | CCGAAAATAAACAAAACTGAAGGACAGCCGATTTACAAG |
| | | GTAGGGACACCATGCGACGATTGCAGTGAATACACAAA |
| | | AAAAGCAGACAATACCACGTCTGCGGATCCGGTGTGTA |
| | | TTCCGGATGACGGAGTCTGCTTTATTGGCTCGAAAGCC |
| | | GATTACGATAGCAAGGAGTTTTATCGATTCCGAGAGTTA |
| | | TGA |
| 38 | ANCCAN_12561 | ATGACTGATACTCTACGAGATGAATACGTCAGGCTGCAC |
| | | AATTTCCGAAGGGGCCTACTCGCAAAAGGGCAAATTCC |
| | | TAGAAAGGATGGGAAGTATCTACCAATGGCTTCTAACAT |
| | | GATGGCGATTACATATGACTGCAGCTTAGAAGAAGGAG |
| | | CCATATCGTGGGCTGCACAGTGTCCCAAAGCAAGTTCA |
| | | CCGCAAAGCTTTAGACCCGGATTGGGAGAGAATTGCTA |
| | | CTCATTCCCGGCGACAAGGTTCAACTTCGACACAGCAG |
| | | CTAAAAAATCTGTTACTGAATGGTGGAAGCCGATTCGTG |
| | | ACGTGAATTACTTCGAAAAGGTCGTCGTATTCCGTCCAT |
| | | TCCATGAAGGTGCACCGATATCCACCTTCACACAGATG |
| | | GGATGGGCTACAAGTAACAAGTTAGGATGTTCAATCGTG |
| | | AAATGCACGACGTCCAACCGATACGTAGGCGTTTGCCG |
| | | CTATTCGCCGAGGTACTCACTTATTCGTTCTCCCTGA |
| 39 | ANCCAN_11005 | AGACCGGTGACATTCGTCCAACCACAATGTCAGCCAAA |
| | | CGGTTACCTAGACAAGACTACAATTGACAACGGCATTCT |
| | | TCAACCCATCAATGATGAAAGAGCAAAAGTGGCTAAACA |
| | | CGAGCAACAAAATGGCATGAGCCCTACACAGAAACTGC |
| | | CACCAGCAACGGGAATGACTCAGTTGGTCAGTTGTTTTC |
| | | TGAGTGGATGA |
| 40 | ANCCAN_11519 | ATGCAGCCCCCTCCACTCTCCTGCGTTCGCTACTGCAA |
| | | GCATTGGGACTGCAACATAGAGAACTATGCTTCGCTGC |
| | | ATAATTGCAACAACAAAATCGGAATTATGCCAAATTTTTC |
| | | GACAATTTCAACAAACATCAAGCTGACCGGCAAGCCTTG |
| | | CGACATCAATAAAGAGACGAAAACTGTTCTAAATAAATTA |
| | | TGGAATGAGGCAAAGAACGCGGATCTGAAAGATCCAAA |
| | | GTACAATGACGCTACAATAAAGAATTTCGGCATAATGGC |
| | | TTACGGGAACACCACTGGTTTCGCGTGCACCTACAACA |
| | | AAGAGTGTTCTGACAATCTGCTCTGTTGGTACAATCGAA |
| | | AGCCGGAGAACGGCAAGCCTCTTTACACATCTGGCTCC |
| | | GACTGTACTAAGGGTGGCTGCACAAACAGCACCTGTGT |
| | | CGACTATCTGTGCCAACTAAAAGAATACACCCCAACTAA |
| | | GGACTTTCTTCCAAGATACTGCCACCCGGACGTCGACG |
| | | GATTGACCTACGAACAACACCTTACAGCTCAGTACATGA |
| | | TGAACTATTACAGAAGACTTCTTGCGACCGGATGGACTC |
| | | GAGACAAGAGCGCTTATGCGCCAACAGCGACAGCGTTG |
| | | TGGTCAGTGGTTTACCTCTGTGCTTCAATTGGAAAAGCA |
| | | ACCAAAGCATTAGCAGATGCGGTAGCATGCCTGGATCC |
| | | ACCATTTCCGGTCGCGAGAGGTTACACACAGACTCATC |
| | | ACGTTATCAACAGACTTAATGTTGACCCGAAAGAAGCTC |
| | | TCGAAGAGGCAATCAAAGCATGGGCGGAACAGGCAAAA |
| | | AAAGTGGATCTCAAACCAATAGGAGGGGCAGTTTTCTAC |
| | | CAGGATGAAGTCGAAGAGAAAGCCAGCGATTTTGCAAA |
| | | AATGATAACCGACAGAAATTCAGCATTTGGATGCGGCAT |
| | | ATCGGAGTGCAAGGCGAAGAGTTACACACTTGTCTTATG |
| | | TCATTTTAACGGGCTCTTTACTTCTCGCGATCCAATCTAC |
| | | ACTGTCGGCAAGCCATGTTCGCGCTGCGCTAAATTTAAT |
| | | AAGAAGTGCGAAGATGCTCTTGGCGGAGGGTTATGTAT |
| | | CAACAAATAA |

SEQ ID NOs:41-53 encode the proteins of SEQ ID NOs:1, 3-6, 8, 9, 11-13, 17, 18 and 20, respectively, and are set out in Table 4. SEQ ID NOs:41-53 incorporate codon sequences optimised for mammalian expression constructs and incorporate the signal peptide MLVLVPLLALLAVSVHG (SEQ ID NO:69), namely ATGCTGGTGCTGGTGCCACTGCTGGCCCTGCTGGCCGTGTCCGTGCACGGC (SEQ ID NO:70). It will be understood that SEQ ID NOs:41-53 may include or not include this signal peptide. Accordingly, in some embodiments, SEQ ID NOs: 41-53 do not include signal peptide MLVLVPLLALLAVSVHG, namely ATGCTGGTGCTGGTGCCACTGCTGGCCCTGCTGGCCGTGTCCGTGCACGGC, in the nucleotide sequence.

TABLE 4

| SEQ | | Nucleotide sequence encoding |
|---|---|---|
| ID | Gene name | recombinant protein |
| 41 | ANCCAN_22177 | ATGCTGGTGCTGGTGCCACTGCTGGCCCTGCTGGCCGT |
| | | GTCCGTGCACGGCCAGGCACCAGACTACCAGTGCTGG |
| | | AACTTCGGCCAGACCAATGAGATCCGGCACATCTATCT |
| | | GTCTGAGGTGAACAAGCTGAGACAGCCTATCGGCGAGG |
| | | GCAAGGCCCCAAACAAGGATGGCATGTGCCCCCAGGG |
| | | CAAGAATATCTACAAGCTGTTTTGGGACTGTATCCTGGA |
| | | GAATTATGCACAGGAGGCAGTGGATAAGTGCGGAGCAA |
| | | AGCCAACCGTGCCTGCCGAGCTGTCTATGGTGTGGAAC |
| | | GAGACCACACTGACCACATGTAATCCTACACCCAGCTTC |
| | | AAGAAGCAGGTGAACGACTGGTGGGATGAGGTGAAGA |
| | | GGGTGGGACTGGGCACCGACGCAGCCTTCAAGCCAGG |
| | | CCTGGAGAACTTTGCCGTGCTGGCCAATGGCCTGGCCA |
| | | CAAGAATCGGCTGCGCCCAGAAGAACTGTAATGGCAAG |
| | | CTCCAGATGGCCTGCGTGGTGTACGGCAAGGCAGCCA |
| | | GCAATGCAGGACAGGCAATCTATGAAGTGGGAGCAGGC |
| | | TGCTCCGGCGACCAGGATTGTACCACATACGCCGGCTC |
| | | CAAGTGCCTGACCAAGAAGAAGTTTTGCGTGGCAGGCT |
| | | ATCCCACCAGCGCCGTGGTGGTGCCCCCTACAGTGGG |
| | | CACCACAGAGGCCATGACCAGCTCCACACAGTCTACAA |
| | | GCACCTCCACATCTACCAGCACACAGCCCGAGAAGACC |
| | | ACAACCTCCGAGAAGCCTTCCACATCTGCCACCGGCAC |
| | | ATCCAACATCTGTCCTCACTCTCCACCCGACGCCAATAA |
| | | GGATCTGGCCAGGGAGGAGCTGCTGAAGGTGCACAAC |
| | | GAGAAGAGGGCAGCAATCGCAAAGGGAGAGGTGAAGA |
| | | TGGGCAATGGCAAGATGGCCCGGAGATGCCCACGGAT |
| | | GAAGAAGTTCGTGAGATACAACTGTACCCTGGAGGTGG |
| | | CCGCCTATGAGAAGGCCAAGACCTGCCCAACAGCCGAC |
| | | CCCAACATCGAGAACGTGAGCTGTTTCCTGAGCAACGC |
| | | CAGGAATAAGAAGCTGGCCGCCCGCGAGGCTATGGAC |
| | | GCATGGTTTGGCGAGATCAGCAAGTCCAACGCACACAT |
| | | GGAGCAGGCAACCGGCAGCCAGAACCTGCTGCTGCCC |
| | | GCCCTGAATATCAGGCAGTTCGCCCGCATGGTGTGGGA |
| | | TAGGAATGCCGAGATGGGCTGCGGCATCGCCGAGTGTA |
| | | AGGGCTCCCACAAGTACGTGGTGGTGTGCCAGTATGGA |
| | | CAGGGAGTGGGCAAGTACGGCGACACCATCTATATGAT |
| | | GGGCCCTACATGCAACCAGTGTGGAGGACCATCTGGCG |
| | | GCCGGTGCACCGATGGCGCCTTTTGTCCTCATCACCAC |
| | | CACCACCACTGAGGATCC |
| 42 | ANCCAN_12569 | ATGCTGGTGCTGGTGCCCCTGCTGGCCCTGCTGGCCGT |
| | | GTCCGTGCACGGCATGTCTTTCACCACATGGCTCCAGC |
| | | AGCCTCACGGCGGCGGAGGAGGCGGCAGGTGCGCAG |
| | | GCTCCGCCACAGCCGACCGCATGACCGATACACTGCG |
| | | GGACGAGTTCGAGAGACTGCACAACTTTCGGAGAGGAC |
| | | TGCTGGCCACCGGACAGATCCCAAGGAAGGATGGCAA |
| | | GTACCTGCCCAAGGCCTCTAATATGCGCAAGATCATCTA |
| | | CGACTGCGCCCTGGAGGAGGGAGCAATCCAGTATGCCT |
| | | CTCAGTGTCCTACCACACACAGCGAGCAGAGCAGCCGG |
| | | CCCAACGTGGGAGAGAATTTCAAGACATTTCCCGCCAC |
| | | CAGATTCACATTTGATACCGCCGCCAAGAAGAGCGTGA |
| | | CCGAGTGGTGGAAGCCCATCCGGGATGTGAACTACTTC |
| | | GAGAAGGTGGTGGTGTTCAGACCCTTTCACGACGGCGC |
| | | CCCTATCTCTAGCTTTACCCAGATGGGATGGGCACTGA |
| | | GCCACCAAGTGGGCTGCTCCATCGTGATGTGCAGCGCC |
| | | TCCAATAGATACGTGGGCGTGTGCCGGTATTCCCCTCG |
| | | CGGCAACATCGTGGACAACAACGTGTACATGGTGGGCA |
| | | ATCCATGCTCTGCCTGTCACAGCGGAGCAACCACATGC |
| | | TCCTCTGCCGAGGGCCTGTGCTTTCATCACCACCACCA |
| | | CCACTGAGGA |
| 43 | ANCCAN_07727 | ATGCTGGTGCTGGTGCCCCTGCTGGCCCTGCTGGCCGT |
| | | GTCCGTGCACGGCATGAACTCTCCCTACGCCACCATCG |
| | | TGGACAATCAGGATTTCCACCCTGCCCTGTATGCCGAG |
| | | AAGATCGATCGGGCCCTGAGAGCCGGCGACAAGGATA |
| | | CCATCGTGAAGGTGCTGACAAGCATCTCCAACAATCAG |
| | | AGGCAGCAGCTGCGCGAGCCTTACAAGATCAAGTATGG |
| | | CAAGGACATCATCGCCGCCCTGGATAAGAAGTTCAGCG |
| | | GCGACCTGGAGCGGACCATCTTTGCCCTGATGGACACA |
| | | CCACTGGACTACGATGTGAAGCAGCTGAAGCAGGCCAT |
| | | GAAGGGCCTGGGCACCGATGAGGCCGTGCTGATCGAG |
| | | ATCCTGTGCTCCAGGACACCAGACCAGATGGCCGCCAT |
| | | CAGGATCGCCTACGAGCGCGAGTATAAGACCCCCCTGG |
| | | AGAAGGACGTGGCCAGCGATACATCCGGCGAGTTTAAG |
| | | GATCTGCTGGTGGCCCTGGTGACCGGCTCTCGGGATG |
| | | GCAGCAGAAACACAAATGACCAGGAGGCCAAGGACGAT |
| | | GCCGTGAGGCTGTACGCCGATGGCAAGGCCAAGCTGG |
| | | TGGGCAAGGACTCTCCCAGCCACTTCCTGAAGATCCTG |
| | | GCCACCCAGAACCAGTATCAGCTGCGCAAGGTGTTCGC |
| | | CGCCTTTGCCGACCTGTCCGGCTCTACCATCGAGAAGG |
| | | CCATCGAGAAGGAGTTTAGCGGCGACCTCCAGAAGTCC |
| | | TACCTGACAATCGTGCAGGCCGCCTCTGACAAGCAGAA |
| | | GTTCTTTGCCCGGCAGCTGTATAACAGTATGAAAGGCCT |
| | | GGGCACCAGAGACAATGATCTGATCCGGGTGCTGGTGA |
| | | CAAGATCCGAGGTGGATCTGGAGCTGATCAAGCAGGAG |
| | | TTCCAGGCCCTGTACAACAAGCCTCTGGAGGACATGAT |
| | | CAAGGGCGATACATCTGGCCAGTATAGGGACGCACTGC |
| | | TGGCCATCGTGGTGGGAAATCGGATGCACCACCACCAC |
| | | CACCACTGAGGATCC |
| 44 | ANCCAN_00478 | ATGCTGGTGCTGGTGCCACTGCTGGCCCTGCTGGCCGT |
| | (Ac-ASP-2) | GTCTGTGCACGGCAACAGCATGAGATGTGGCAACAATG |
| | | GCATGACCGACGAGGCCAGACAGAAGTTCCTGGATGTG |
| | | CACAACTCCTACAGGTCTATGGTGGCCAAGGGCCAGGC |
| | | CAAGGACGCCATCTCCGGAAATGCACCAAAGGCAGCCA |
| | | AGATGAAGAAGATGATCTATGATTGTAACGTGGAGTCTA |
| | | CCGCCATGCAGAATGCCAAGAAGTGCGTGTTCGCACAC |
| | | TCTCACAGGAAGGGAGTGGGAGAGAATATCTGGATGAG |
| | | CACAGCAAGGCAGATGGACAAGGCACAGGCAGCACAG |
| | | CAGGCCTCCGATGGCTGGTTTTCTGAGCTGGCCAAGTA |
| | | CGGCGTGGGCCAGGAGAACAAGCTGACCACACAGCTG |
| | | TGGAATAGGGGCGTGATGATCGGCCACTATACCCAGAT |
| | | GGTGTGGCAGGAGAGCTACAAGCTGGGCTGCTATGTG |
| | | GAGTGGTGTAGCTCCATGACCTACGGCGTGTGCCAGTA |
| | | TAGCCCCCAGGGCAACATGATGAACAGCCTGATCTACG |
| | | AGAAGGGCAACCCTTGCACAAAGGACAGCGATTGTGGC |
| | | TCCAATGCCTCTTGCAGCGCCGGCGAGGCCCTGTGCGT |
| | | GGTGCGGGGACACCACCACCACCACCACTGAGGATCC |
| 45 | ANCCAN_07322 | ATGCTGGTGCTGGTGCCCCTGCTGGCCCTGCTGGCCGT |
| | | GTCTGTGCACGGCAAGGCAATCGCACCTGAGGGAGTG |
| | | GTGAAGGCCGCCGCCGGATACGCCTATGCCGTGGACC |
| | | TGGAGGTGCCCGTGCCTCTGAGCGGCTTCAATTGCATG |
| | | AAGAAGTACGGCTATAAGACCGTGTTTATCAGGGCCTAC |
| | | GACCCAACCGGAGCAGGCAGATTCGATACAAACGCCGT |
| | | GAACAATATCAGGAACGCAAATCAGGCAGGACTGGGCA |
| | | CCGAGGCCTACATGACACCCCAGCTGCACTCTAATAAG |
| | | CGGGGCAGCCAGCAGTTTAGAGAGCTGTATGAGGGCCT |
| | | GAGGAACGCCAAGATCCAGGTGCGCACCGTGTGGGTG |
| | | CAGGTGACAAGCCCAGTGAACTGGGGCCCCAATACACA |
| | | GGCCAACATCTACTTTATCAATGACATCGTGAGCATGGC |
| | | CAAGTACTATGGCGTGACCATCGGCTTCTACACAAACGT |
| | | GTACGATTGGAACCAGATCACCAAGGGAGCAAACGTGG |
| | | AGGGAGCAATGCTGTGGTATTGGAACGTGAATGGAGCA |
| | | GGAGTGAATGGAGAGACACCTGCAAACTTCGACGACTT |
| | | CCGGCCTTTCGCCAAGTTTACCAAGCCAACAGTGAAGC |
| | | AGTTCGGACAGGTGGAGCACCTGTGCGGAGTGACCGT |
| | | GAATCGCGACGTGTATACACTGAACAATAGCATCAACCT |
| | | GGTGAGCAGCAAGGCCGAGAAGCTGGATGGAGAGCTG |
| | | ATCGTGGGCACCCTGGGAGGAAATCAGATGTCCGGCCT |
| | | GGTGCAGTCTGTGCACCACCACCACCACCACTGA |
| 46 | ANCCAN_08034 | ATGCTGGTGCTGGTGCCCCTGCTGGCCCTGCTGGCCGT |
| | | GTCCGTGCACGGCAAGATGCAGAACGTGACCGTGAAG |
| | | GGCACCACAATCTGCAATAAGAAGCGGATGGCCGACGT |
| | | GACCGTGGAGCTGTGGGAGAGAGACACCCTGGACCCC |
| | | AACGACCTGCTGGATAGCAAGAAGACCAGCCGGGAGG |
| | | GAGAGTTCCTGGTGAAGGGAGGCCAGAACGAAGTGGG |
| | | CTCTATCGAGCCCTTTCTGAGGATCACCCACACATGCAA |
| | | TGTGAAGAAGCCTGGCTGTAAGCGCATCACCGAGTTCG |
| | | ACATCCCTAAGTCTAAGATCGACACAGTGTACGATATGA |
| | | CCTATGTGACACTGGACATCATCAGCGCCGTGGATAAG |
| | | GAGAAGTGTCATCATCACCACCACCACTGA |
| 47 | ANCCAN_07062 | ATGCTGGTGCTGGTGCCTCTGCTGGCCCTGCTGGCCGT |
| | | GTCTGTGCACGGCGCCCAGGCACCAAACTACCAGTGCT |
| | | GGAATTTCGGCCAGCAGGACGAGATCCGGAGAGATTAT |
| | | CTGGTGGAGGTGAACAAGCTGAGGGAGACAATCGCAAC |
| | | CGGCACAGCACCATCCAAGGACGGAAGGAAGTGCCCT |
| | | CAGGGCAAGAACATCTACAAGCTGAATTGGGACTGTAT |
| | | CCTGGAGAATTATGCACAGAAGGCCGCCGATGAGTGCG |
| | | CAGAGCCTAAGACCGTGCCCAAGAAGCTGAGCTTCGTG |
| | | TACAACGCCACCACACTGACCACATGCAATCCTATCCCA |
| | | GACATCAAGAAGCAGGTGAACAATTGGTGGTCTGAGGT |
| | | GGAGAGGGTGGGCCTGGCCAAGAACGCAGCCTTCAAG |
| | | CCTAAGCTGCGGAACTTTGCCGTGCTGGCCAATGGCCT |
| | | GGCCACAAGAATCGGCTGCGCCCAGAAGAACTGTAATG |
| | | GCGTGCTGCACATGGTGTGCGTGGTGTACGGCAAGGC |
| | | AGCCAGCTCCACCAGGCAGGCAATCTATGAAGTGGGAG |
| | | CAGGCTGCGAGGGCAATTACCAGTGTACCACATATAAG |
| | | GAGAGCAAGTGCCTGACAAAGAGGAAGCTGTGCGAGG |
| | | CAGGCTACCCCACCTCCGAGGCAGTGGTGCCCCCTACA |
| | | GTGTCTACCAGCAAGCCTACCATCACAAGCACCGAGGC |
| | | AACCCAGCCAGAGCCTCAGCCACCCAAGCCCGAGCCTC |
| | | CAAAGCCTGAGCCCCCTAAGCCAGAGACCACAAAGCCA |
| | | CCCGTGACATCCGAGCACTGCCCCAAGCAGGAGCACAT |
| | | GAACACCGACCTGGCCAGGGAGGAGTTCGTGAACCTG |
| | | CACAATCAGGCCAGAGCCACCATCGCAAAGGGAGGAGT |
| | | GAAGATGGGCGATGGCAAGATGAGCAGGCCATGTCCC |
| | | CGCATGAAGAAGTTCGACATCAAGAATTACGATTGCAAG |
| | | CTGGAGGAGGTGGCCTATGCCACAGCCGCCAAGTGTCA |
| | | GACCGAGGTGTACGTGCAGAACGAGAATTGGTTTATGG |
| | | CCTCTACAAAGGCCACCAACAGGAAGCTGGCCGCCAGA |
| | | GAGGCAGTGGAGCACTGGTATAATGAGATCAAGAGCGG |
| | | CCACATGGAGCAGAAGACCGGCTCCCAGAACCTGCTGA |
| | | TGCCATCTTTCAAGATCGACCACTTTGCCCGGATGGTGT |
| | | GGGATACAAATACCCAGATCGGCTGCGCCGCCCACAAG |
| | | TGTGGCAAGATCTACCACGTGGTGTGCCGGTATGGCCC |
| | | AAAAGTGGGCAAGTACGGCGACACAATCTATATGATGG |
| | | GCCCCACCTGCAACCAGTGTCAGGGCAGGTGCATCGA |
| | | GGGCGCCTTTTGTCCCCACCACCACCACCACCACTGAG |
| | | GATCC |
| 48 | ANCCAN_10127 | ATGCTGGTGCTGGTGCCCCTGCTGGCCCTGCTGGCCGT |
| | (Ac-FAR-2) | GTCCGTGCACGGCGCTCCTAACGTGGAAGTGAATAAGT |
| | | TCGAGGACATCCCCGAGCAGTACAGGGAGCTGATCCCT |
| | | AAGGAGGTGGCTGATCACCTGAAGGCCATCACCGACGA |
| | | TGAGAAGAAGATCCTGAAGGAGGTGCTGAAGGACTACG |
| | | CTAAGTATAAGGATGAGAACGAGTATCTGGCCGTGCTG |
| | | AAGGAGAAGTCCCCTAGCCTGCACGAGAAGGCTAAGAA |
| | | GTTCCATGAGTTTATCAAGGCTAAGGTGGACGCCCTGG |
| | | GCGATGAGCCAAAGGCCTTCGTGAAGAAAGTGATCGCC |
| | | GCTGCCAGGAAGCTGCACGCTGAGCTGCTGGCCGGCA |
| | | ACAAGCCCTCCCTGGAGGAGCTGAAGAATACCGTGAAG |
| | | ACATACATGGCTGAGTTTAACGCCCTGTCTCCAGCTGCC |
| | | AAGGAGGACCTGAAGAAGAACTTCCCCATCCTGACCTC |
| | | CGTGTTTACAAATGAGAAGGCTAAGGCCCTGATGGATAA |
| | | GCACCTCCAGAATGAATTCTGCAGATATCCAGCACAGTG |
| | | GCGGCCGCTCGAGTCTAGAGGGCCCCATCATCACCATC |
| | | ACCAT |
| 49 | ANCCAN_01926 | ATGCTGGTGCTGGTGCCTCTGCTGGCCCTGCTGGCCGT |
| | | GAGCGTGCACGGCATGAAGAAGGCCGTGAAGTGGGGA |
| | | CTGCTGGCCGCCGTGGTGGTGGCCGTGATCGCAGCAA |
| | | TCGTGGCAGTGGTGCTGGTGCTGGTGCTGAAGAAGAAG |
| | | GATGAGGGCCCATCTGAGAGAGTGATGTACTTTGCAAT |
| | | GCACCTGGGCGACGATGGCGCCCTGTATCGGAATAGAT |
| | | ACGAGGATTATAAGAGCGGCATGTGCAACCTGAAGATG |
| | | AATGTGGACAACACCCGCAGCTCCATCAAGGCCATGAA |
| | | GTCCCTGACACAGAAGTACTCTTTCATCCTGTTTACCGA |
| | | TAAGATCGAGACCACAGCCCCAATGTCTAGCGACGAGG |
| | | CCATCCAGAAGCTGGGCTCCATCGAGCCTAAGGCCCAC |
| | | GAGTCTCCATTCAATCAGGAGGCCGTGCTGAAGGAGTT |
| | | TACCGGAAGGGGAGGCGACAACCTGCTGGTGTACTATA |
| | | TCCCCTGTAGCTTCGACTACGATTCCGAGAAGGTGAAG |
| | | CAGTTTGTGGGCGAGATGAAGAAGGCCGGCCTGGAGA |
| | | AGAAGATCCTGATCGTGAGCAATACCAGGACAGAGGGC |
| | | GAGATCCGCCAGCTGTATGAGCAGAACAATGTGGCCGG |
| | | CTCCGATACAGAGAACGTGGCCGAGAAGATCATCGACT |
| | | TCGGCCACAGCGAGTCCGAGAAGACCACACTGGAGCC |
| | | CAAGACCTCTCCTAAGCCAGGCACCACAAAGACCACAC |
| | | CCCAGAAGCCTAGCCAGGAGCCAACCAAGCCAACAAGG |
| | | CCTACCAAGCCCCCTGGCCCCTCCCCTAACCCAAATGG |
| | | CCTGCACTGCCTGTTCGTGGGCGATCTGTACAATTTTGG |
| | | CACCCGGACAGAGAACTATGACAAGGAGGCCGCCCTGT |
| | | TCGGCGACATCGGCTACGATTTCTTTGAGGAGTCCTCTA |
| | | TCTCCAGAATCGGCCTGTGGGCCTACGGCCACACAAAT |
| | | TATCCAAAGAAGCCCGATCTGTCCAACATGAACAGCAAC |
| | | TTCGAGGACTTTTATTCTGATCTGAACAACATGGAGTTT |
| | | CAGAATACCAAGGACCCTATGAACACCGAGACAGCCAT |
| | | CAGAGTGATCAATGGCCTCCAGGACGATGGCAGCATCG |
| | | TGAACTGCCTGGTGTTCTTTTCCGCCAGACAGGATACAC |
| | | AGTCTCTGCCCAAGCTGGACCCTCAGAACAAGAAGTTC |
| | | AAGAGGATCGTGGCCGTGGGCTACAACAATACCGATCT |
| | | GTCTGGAGTGGTGGGCAGCCGCGGCGTGGCCGTGTCC |
| | | GTGCCCTACCACTATCTGGACGGCGATGTGGCCACAGT |
| | | GCTGGACGCCATCATGGGCAGACCCGTGACCAAGACCA |
| | | CACCTAAGACAGTGCCCACCCCTAGGCCAACCACAAGC |
| | | ACCGAGGTGCCACCTATGGACAAGTCCAAGTGTCTGCT |
| | | GGTGGGCGACCTGTACAACTTCGGCAGGGAGGACGAG |
| | | AAGTATGACATCGAGGCCGACCTGATCTCTAAGATCGG |
| | | CTACGATTTCCTGCTGGGCAGCGACGGCGACCTCCAGC |
| | | TGGCCCTGTGGGCCTACGGCTATACACAGTTTAGCAAG |
| | | ACCGTGAATGACTCCCTGAAGGCCATGCGGAAGAACTA |
| | | CGAGGAGTTCGAGCCAGACCTGGATAAGCTGGACTTTA |
| | | AGGATGTGAGCAATCCCTGGAGCAGCGAGTCCGCCATC |
| | | AAGGCCATCAACGAGATGTATGATAGCCAGAAGAGAGT |
| | | GGACTGTCTGGTGTTCTTTTCTGCCCAGCAGAATCCACA |
| | | GAGCCTGCCCAAGCTGAATCCTAAGAACACCAAGTTCC |
| | | GCAAGATCATCGCCGTGGGCTTTAACAATGTGGACCTG |
| | | AGGCACATCGTGCCAGCAAACGGCCTGGCCTTCAAGGT |
| | | GCCTTTTCACTACCTGGACGAGGACATCACCCCCATCG |
| | | TGAAGGCCATGCACGGCCGCTATGTGCCAAAGACCACA |
| | | GTGAAGCCCACCACAAAGAAGACCACAACCACAGCCCC |
| | | TCCACCCCCTAAGAAGCTGAATTGTCTGTTCGTGGGCG |
| | | ATATGTACAACTTTGGCAGCATCCTGGACCACATCGAGG |
| | | GCGAGGTGCAGTTCATGTCCGATCTGGCCTACGACTTC |
| | | TTTGGCCAGGGCGAGAATTGGAACGCCGGCCTGTGGG |
| | | CCTATGGCTTCACACACTTTCCTACCCTGCCAAATCTGA |
| | | AGAACATGAAGAACAACTACAAGGACTTCGAGAAGGAG |
| | | CTGGGCACAATGGAGTTTACCGACCGCGATAACTATCT |
| | | GACCACAAGCAAGGCCATCGAAGTGATCAACCGGCTGA |
| | | TGGACGATGGCTCTAGCGCCAATTGTCTGGTGTTCTTTT |
| | | CCGCCCAGAAGAATACCAACATCCTGCCAAGACTGTCT |
| | | CCCATCTACAAGAAGTGGGAAAGAATCGTGGCCGTGGG |
| | | CTTCAATGACACAGATCTGTCCGGAGTGGTGTACGGCA |
| | | CCGGAGTGGCCGTGAGGGTGCCCTTTCACTATCTGGAT |
| | | GAGCACGTGAACAAAGTGATGGACGCCATCATGGGCCG |
| | | GTATGTGCCTCAGACCACAAAGAGACCAGTCACAGTGC |
| | | CCACTCCTAGGCCAACCACACCTGTGCCACCCGGCACA |
| | | GGCCAGAATTGTCTGCTGGTGACCGACATGTCCAACTTT |
| | | GGCCGCGACATGGATAAGTACGAGATCGAGCTGGATCT |
| | | GCTGTCTAAAGTGGGCTATGACTTCTTTGAGAAGTCTAA |
| | | TAAGAGCGTGGTGGCCCTGTGGGCCTACGGCTACACC |
| | | GATTACTCTAAGGACGTGGCCAAGAGCCTGGAGAAGCT |
| | | GTTCCCTAACTATCGCCAGCTGGCCACAGAGCTGGAGC |
| | | GGATGAAGCTGCACGAGACCTCCTCTCCACTGACCACA |
| | | GCCAAGCACCACCACCACCACCACTGAGGATCC |
| 50 | ANCCAN_19762 | ATGCTGGTGCTGGTGCCCCTGCTGGCCCTGCTGGCCGT |
| | | GTCCGTGCACGGCCAGTCTCTGATCCCTTTCGGCATGG |
| | | AGATGTGGTACTGCCACAGCGTGCCAAGGTCCGGACCA |
| | | CAGCTGAGGAACGTGTTCCTGGACCTGTGCCCTAATAG |
| | | CGCCTTTGATAAGGAAGTGATCGAGGAGCTGGTGCTGG |
| | | GCCACATCAACGACCAGCGGTACAAGCTGCTGAGAGGA |
| | | CTCCAGCTGAACGGACCTTGGCAGGGCAAGGATAAGTG |
| | | GACCCCAGAGACACAGGGCTATGGCAAGAAGCTGCCAA |
| | | CCGGCAAGACAATGAACAAGCTGGAGTGGTCCTGCAAG |
| | | CTGGAGAAGAATGCCAAGGCCGCCCTGGACCCCAAGT |
| | | GTACCGATGTGGCACCAAACGCACCTCAGGGCAAGACA |
| | | GGCCTGTTTTATAAGGCCGACATCGATTGGGAGGTGCC |
| | | CGACATCACCTCTGCCGCCTGGGCATGGATGGAGCAGA |
| | | TCGAGAAGTTCGCCGTGAGCGACGATGCCATCTCCGAT |
| | | AAGCAGGTCATCTTCAAGGACCACGATCAGCACCACTC |
| | | TAAGACCACAATCTGGTGCATCAGGCTGGGCAAGGTGG |
| | | CAGCAACCAAGGAGAGGACAGTGCGCATGGACAGCCA |
| | | CGCCAAGATGGAGTGCGTGCGGAAGCAGACACACCGG |
| | | AGAGGCCTGCACTCTCTGATGAAGAGCCACCACCACCA |
| | | CCACCACTGA |
| 51 | ANCCAN_04194 | ATGCTGGTGCTGGTGCCCCTGCTGGCCCTGCTGGCCGT |
| | (Ac-NIF) | GTCTGTGCACGGCAACGAGCACAATCTGAGGTGCCCAC |
| | | AGAACGGCACCGAGATGCCTGGCTTCAATGATAGCATC |
| | | CGGCTCCAGTTCCTGGCCATGCACAACGGCTACAGATC |
| | | CAAGCTGGCCCTGGGCCACATCTCTATCACAGAGGAGA |
| | | GCGAGTCCGACGATGACGATGACTTCGGCTTTCTGCCA |
| | | GATTTCGCCCCCAGGGCCTCCAAGATGCGCTACCTGGA |
| | | GTATGACTGCGAGGCCGAGAAGTCTGCCTATATGAGCG |
| | | CCCGGAACTGTTCTGATAGCTCCTCTCCCCCTGAGGGC |
| | | TACGACGAGAACAAGTATATCTTCGAGAACAGCAACAAT |
| | | ATCTCTGAGGCCGCCCTGAAGGCCATGATCTCCTGGGC |
| | | CAAGGAGGCCTTTAACCTGAATAAGACCAAGGAGGGCG |
| | | AGGGCGTGCTGTACAGGAGCAATCACGACATCTCCAAC |
| | | TTCGCCAATCTGGCCTGGGACGCCCGCGAGAAGTTTGG |
| | | ATGCGCAGTGGTGAACTGTCCTCTGGGCGAGATCGATG |
| | | ACGAGACCAATCACGACGGCGAGACATACGCCACCACA |
| | | ATCCACGTGGTGTGCCACTATCCAAAGATCAACAAGACA |
| | | GAGGGCCAGCCAATCTACAAAGTGGGCACCCCCTGCGA |
| | | TGACTGTAGCGAGTATACAAAGAAGGCCGATAATACCAC |
| | | CAGCGCCGACCCCGTGTGCATCCCAGATGACGGCGTG |
| | | TGCTTTATCGGCTCTAAGGCCGATTACGACAGCAAGGA |
| | | GTTCTATCGGTTTAGAGAGCTGCACCACCACCACCACC |
| | | ACTGAGGATCC |
| 52 | ANCCAN_12561 | ATGCTGGTGCTGGTGCCCCTGCTGGCCCTGCTGGCCGT |
| | | GTCCGTGCACGGCATGAACGCCATCACATGGTCTTGCG |
| | | ACCTGGAGAAGGTGACCCAGGAGGGCCTGAAGAGCCA |
| | | CTCCTGTGACTCCAACTTCCCACCTGTGGACCCCCAGG |
| | | GAAGGGCCCAGCTGTACACATCTGCCGATAATGAGTAT |
| | | GAGAGCACCGTGGAGGAGTTTATCGCCTCCCGCGTGGA |
| | | CCTGATCGATTCTAATGCCCTGAAGGACGTGGGCCCAA |
| | | ACAGCGTGAAGTACACCGATGCCACAGCCACCTCTGAG |
| | | TATAGCAACGTGATGAGGGCCACCACAACCCAGATCGC |
| | | CTGCACAAAGAAGGTGTGCACCGACATCAATATGTACGT |
| | | GATCTATTGCATCACAAATCAGAAGGCCCTGACCAACG |
| | | GCGAAGTGATCTACGAAGTGGGAATCGCAGGACAGTGC |
| | | ACCGACTGTCCAGCCGGCACAGTGTGCGATCCCACCTC |
| | | CAAGCTGTGCGTGGCCTCCGGAGGCACAGTGTCTGCC |
| | | GGCACCGCCAGCACAACCCCTGCAACAACCGCAACAGC |
| | | CGCCTCCACAACCCTGCCAACCTCTCCCGAGGCCAATT |
| | | TCCCTTCTGGCGCCAGCTCCAAGTGCCCAAACAGCCGG |
| | | ATGACAGACACCCTGCGGGATGAGTATGTGAGACTGCA |
| | | CAACTTTCGGAGAGGCCTGCTGGCCAAGGGCCAGATCC |
| | | CCAGAAAGGACGGCAAGTACCTGCCTATGGCCAGCAAT |
| | | ATGATGGCCATCACCTATGATTGCAGCCTGGAGGAGGG |
| | | AGCCATCTCCTGGGCAGCCCAGTGTCCTAAGGCCTCTA |
| | | GCCCACAGTCTTTCCGGCCCGGCCTGGGAGAGAATTGC |
| | | TACAGCTTTCCAGCCACAAGATTCAACTTTGACACCGCC |
| | | GCCAAGAAGTCCGTGACAGAGTGGTGGAAGCCCATCAG |
| | | GGATGTGAATTATTTCGAGAAGGTGGTGGTGTTCCGCC |
| | | CATTTCACGAGGGCGCCCCCATCAGCACCTTCACCCAG |
| | | ATGGGCTGGGCCACCAGCAATAAGCTGGGCTGCTCCAT |
| | | CGTGAAGTGTACAACCAGCAACAGATACGTGGGCGTGT |
| | | GCCGGTATTCCCCCAGAGGCAATATCGTGAACAATAAC |
| | | GTGTACCAAGTGGGCACCCCTTGCAATGCCTGTCCAAG |
| | | CGGCACAACCTGCAACTCCTCTGAGGGCCTGTGCTTCC |
| | | ACCACCACCACCACCACTGA |
| 53 | ANCCAN_11519 | ATGCTGGTGCTGGTGCCTCTGCTGGCCCTGCTGGCCGT |
| | | GTCTGTGCACGGCACCGCCTTCGGCTGTAATGACCGGC |
| | | TGATCACAGATGAGTGGAGAGCCATGATCCTGAAGTTTC |
| | | ACAACAATCACAGGAGAACCGTGGCACAGGGAAACCAG |
| | | GCAATGAAGGGAGGCAAGATGCCCCCTGGCAACGACAT |
| | | CAATGAGCTGCACTGGGATTGTAACATCGAGAATTACGC |
| | | CTCCCTGCACAATTGCAACAATAAGATCGGCATCATGCC |
| | | TAATTTCTCTACCATCAGCACAAACATCAAGCTGACCGG |
| | | CAAGCCATGCGACATCAACAAGGAGACCAAGACAGTGC |
| | | TGAACAAGCTGTGGAATGAGGCCAAGAACGCCGACCTG |
| | | AAGGACCCCAAGTATAATGATGCCACCATCAAGAACTTC |
| | | GGCATCATGGCCTACGGCAATACCACAGGCTTTGCCTG |
| | | CACATATAATAAGGAGTGTAGCGACAACCTGCTGTGCTG |
| | | GTACAATCGGAAGCCTGAGAACGGCAAGCCACTGTATA |
| | | CCAGCGGCTCCGACTGCACAAAGGGCGGCTGTACCAA |
| | | CTCCACATGCGTGGATTACCTGTGCCAGCTGAAGGAGT |
| | | ATACCCCAACAAAGGACTTTCTGCCCAGGTACTGTCACC |
| | | CTGACGTGGATGGCCTGACCTACGAGCAGCACCTGACA |
| | | GCCCAGTATATGATGAACTACTATAGGCGCCTGCTGGC |
| | | CACCGGATGGACACGCGATAAGAGCGCCTACGCCCCAA |
| | | CCGCAACAGCCCTGTGGAGCGTGGTGTACCTGTGCGC |
| | | CTCCATCGGCAAGGCAACCAAGGCCCTGGCCGACGCA |
| | | GTGGCCTGCCTGGACCCCCCCTTCCCCGTGGCCAGGG |
| | | GCTACACCCAGACACACCACGTGATCAACCGCCTGAAT |
| | | GTGGACCCTAAGGAGGCCCTGGAGGAGGCCATCAAGG |
| | | CCTGGGCAGAGCAGGCCAAGAAGGTGGACCTGAAGCC |
| | | AATCGGAGGAGCCGTGTTTTATCAGGATGAGGTGGAGG |
| | | AGAAGGCCTCTGACTTCGCCAAGATGATCACCGATAGA |
| | | AATAGCGCCTTTGGCTGCGGCATCTCCGAGTGTAAGGC |
| | | CAAGTCTTACACACTGGTGCTGTGCCACTTCAACGGCCT |
| | | GTTCACCAGCCGGGACCCCATCTATACAGTGGGCAAGC |
| | | CCTGCTCCCGGTGTGCCAAGTTCAATAAGAAGTGTGAG |
| | | GACGCACTGGGAGGAGGACTGTGCATCAACAAGCACCA |
| | | CCACCACCACCACTGA |

In some embodiments, the nucleotide sequence encoding the recombinant protein may have at least 70%, at least 80%, at least 90%, at least 95%, at least 98%, or at least 99% sequence identity to any one of SEQ ID NOs:21-53, especially any one of SEQ ID NOs: 21, 24, 26, 28 and 31, more especially any one of SEQ ID NOs: 24, 28 and 31, even more especially any one of SEQ ID NOs: 24 and 28. For example, the nucleotide sequence may have at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, at least 90% sequence identity to any one of SEQ ID NOs:21-53, especially any one of SEQ ID NOs: 21, 24, 26, 28 and 31, more especially any one of SEQ ID NOs: 24, 28 and 31, even more especially any one of SEQ ID NOs: 24 and 28.

In some embodiments, the nucleotide sequence encoding the recombinant protein is any one of SEQ ID NOs:21-53, that is, the nucleotide sequence has 100% sequence identity to any one of SEQ ID NOs:21-53, especially any one of SEQ ID NOs: 21, 24, 26, 28 and 31, more especially any one of SEQ ID NOs: 24, 28 and 31, even more especially any one of SEQ ID NOs: 24 and 28.

In some embodiments, the recombinant protein is encoded by a nucleotide sequence having at least 70% sequence identity to any one of SEQ ID NOs:21-28, especially any one of SEQ ID NOs: 21, 24, 26 and 28, more especially any one of SEQ ID NOs: 24 and 28. In these embodiments, the nucleotide sequence encoding the recombinant protein may have at least 70%, at least 80%, at least 90%, at least 95%, at least 98%, or at least 99% sequence identity to any one of SEQ ID NOs:21-28, especially any one of SEQ ID NOs: 21, 24, 26 and 28, more especially any one of SEQ ID NOs: 24 and 28. For example, the nucleotide sequence may have at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, at least 90% sequence identity to any one of SEQ ID NOs:21-28, especially any one of SEQ ID NOs: 21, 24, 26 and 28, especially any one of SEQ ID NOs: 24 and 28.

In some embodiments, the nucleotide sequence encoding the recombinant protein is any one of SEQ ID NOs:21-28, that is, the nucleotide sequence has 100% sequence identity to any one of SEQ ID NOs:21-28, especially any one of SEQ ID NOs: 21, 24, 26 and 28, more especially any one of SEQ ID NOs: 24 and 28.

Biologically active fragments and variants of the protein of the invention as described herein are also provided.

As used herein, a “biologically active fragment” or “fragment” is intended to refer to a portion of a protein of the invention, including a domain thereof, that retains substantially similar functional activity or substantially the same biological function or activity as the polypeptide, as shown in assays disclosed herein. As used herein, the term “domain” is intended to refer to a part of a protein that shares common structural, physiochemical and/or functional features, for example hydrophobic, polar, globular, helical, or netrin-like (NTR) domains, or other properties, for example a protein-binding domain, a receptor-binding domain, a co-factor binding domain, and the like.

As used herein, a “biologically active variant” or “variant” is intended to encompass proteins or polypeptides having an amino acid sequence sufficiently similar to the amino acid sequence of a protein of the present invention. The term “sufficiently similar” in this context means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, such variants will be sufficiently similar to the amino acid sequence of the preferred proteins of the invention. Such variants generally retain the functional activity of a protein of the present invention. Variants include proteins or polypeptides that differ in amino acid sequence due to mutagenesis.

The term “variant” is also intended to encompass a peptidomimetic of a protein of the invention as described herein. A protein or polypeptide that contains at least one residue that is not naturally synthesised may be referred to as a “peptidomimetic”. A peptidomimetic as used herein is a synthetic chemical compound that has substantially the same structure and/or functional characteristics of a protein of the invention as described herein. Non-natural components of peptidomimetic compounds may be according to one or more of: a) residue linkage groups other than the natural amide bond (‘peptide bond’) linkages; b) non-natural residues in place of naturally occurring amino acid residues; or c) residues which induce secondary structural mimicry, i.e., to induce or stabilise a secondary structure, e.g., a beta turn, gamma turn, beta sheet, alpha helix conformation, and the like. Peptidomimetics can be synthesised using a variety of procedures and methodologies described in the scientific and patent literatures, e.g., Organic Syntheses Collective Volumes, Gilman et al. (Eds) John Wiley & Sons, Inc., NY, al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

The term “variant” is further intended to encompass an ortholog of a protein of the invention as described herein. As used herein, the term “ortholog” is intended to refer to an ortholog of a protein of the invention as described herein from another intestinal helminth (i.e., hookworms, whipworms and roundworms), including but not limited to human hookworms such as Necator americanus, Ancylostoma duodenale and Ancylostoma ceylanicum and pig whipworms such as Trichuris suis and Trichuris trichiura.

As used herein, the terms “substantially similar functional activity” and “substantially the same biological function or activity” each mean that the degree of biological activity is within about 50% to about 100% or more, within about 80% to about 100% or more, or within about 90% to about 100% or more, of that biological activity demonstrated by the protein to which it is being compared when the biological activity of each protein is determined by the same procedure or assay. The biological activity may be evaluated using standard testing methods and bioassays recognisable to those skilled in the art as generally being useful for identifying such activity.

“Similarity” between two proteins is determined by comparing the amino acid sequence of a first protein to the sequence of a second protein. An amino acid of one protein is similar to the corresponding amino acid of a second protein if it is identical or a conservative amino acid substitution.

“Conservative amino acid substitutions” include those described in Dayhoff, M. O., ed., The Atlas of Protein Sequence and Structure 5, National Biomedical Research Foundation, Washington, D.C. (1978), and in Argos, P. (1989) EMBO J. 8:779-785. For example, amino acids belonging to one of the following groups represent conservative changes or substitutions:
Ala, Pro, Gly, Gln, Asn, Ser, Thr:
Cys, Ser, Tyr, Thr;
Val, Ile, Leu, Met, Ala, Phe;
Lys, Arg, His;
Phe, Tyr, Trp, His; and
Asp, Glu.

Other conservative amino acid substitutions may also be made by another one of the same class, the classes being as follows:
Non-polar: Ala, Val, Leu, Ile, Pro, Met, Phe, Trp
Uncharged polar: Gly, Ser, Thr, Cys, Tyr, Asn, Gln
Acidic: Asp, Glu
Basic: Lys, Arg, His

Other conservative amino acid substitutions may also be made as follows:
Aromatic: Phe, Tyr, His
Proton Donor: Asn, Gln, Lys, Arg, His, Trp
Proton Acceptor: Glu, Asp, Thr, Ser, Tyr, Asn, Gln

In some embodiments, a biologically active fragment of a protein of the invention constitutes less than about 225, less than about 200, less than about 175, less than about 150, less than about 125, less than about 100, less than about 75, less than about 50, or less than about 25 contiguous amino acids of an amino acid sequence of a protein of the invention as described herein. Multiple fragments of proteins of the invention are also contemplated.

In some embodiments, a biologically active variant of a protein of the invention may share at least about 70%, at least about 75%, at least about 80%, at least about 85%, at least about 90%, or at least about 95% sequence identity with a reference amino acid sequence such as any one of SEQ ID NOs:1-20.

In order to determine percent sequence identity, optimal alignment of amino acid and/or nucleotide sequences may be conducted by computerised implementations of algorithms (e.g., Geneworks program by Intelligenetics; and GAP, BESTFIT, FASTA and TFAFASTA in the Wisconsin Genetics Software Package Release 7.0, Genetic Computer Group, WI, USA) or by inspection and the best alignment (i.e., resulting in the highest percentage homology over the comparison window) generated by any of the methods selected. Reference may also be made to the BLAST family of programs, for example as disclosed in Altschul et al (Nucl, Acids Res. 25:3389-402, 1997). A detailed discussion of sequence analysis can be found in Unit 19.3 of CURRENT PROTOCOLS IN MOLECULAR BIOLOGY Eds. Ausubel et al (John Wiley & Sons Inc NY, 1995-1999).

The proteins of the invention (and fragments and variants thereof) may be produced by any suitable method known to those skilled in the art. Examples of suitable methods include but are not limited to chemical synthesis (e.g., solid phase peptide synthesis), use of recombinant expression systems (e.g. recombinant DNA technology), and isolation, purification and/or enrichment from a naturally occurring source or from a recombinant source.

In some embodiments, the proteins of the invention (including fragments and variants thereof) are produced by chemical synthesis. Chemical synthesis methods are known in the art. Examples of suitable chemical synthesis methods include those described in Chapter 18 of CURRENT PROTOCOLS IN PROTEIN SCIENCE Eds Coligan et al (John Wiley & Sons NY, 1995-2001).

In preferred embodiments, the proteins of the invention (including fragments and variants thereof) are produced by using recombinant DNA techniques, including cell-based and cell-free technologies. Recombinant techniques are known in the art. Examples of suitable recombinant techniques include those described in Sections 16 and 17 of Sambrook et al (MOLECULAR CLONING. A Laboratory Manual, Cold Spring Harbor Press, 1989), Chapters 10 and 16 of CURRENT PROTOCOLS IN MOLECULAR BIOLOGY Eds. Ausubel et al (John Wiley & Sons Inc NY, 1995-1999) and Chapters 1, 5 and 6 of CURRENT PROTOCOLS IN MOLECULAR BIOLOGY Eds. Coligan et al (John Wiley & Sons Inc NY, 1995-1999).

In some embodiments, the proteins of the invention or fragments or variants thereof) may be modified to enhance suitability for administration, i.e., by the covalent attachment of any type of molecule to the composition such that covalent attachment does not prevent the activity of the composition. For example, but not by way of limitation, derivatives include compositions that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc. Additionally, the derivative can contain one or more non-proteinogenic amino acids

In some embodiments, the proteins of the invention or fragments of variables thereof may be modified to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

In embodiments where the proteins of the invention (including fragments or variants thereof) are prepared by recombinant DNA technologies, the protein may be prepared from a nucleic acid sequence encoding the protein.

### Nucleic Acids

Accordingly, the present invention provides a nucleic acid comprising a nucleotide sequence encoding the protein of the invention as described herein. Suitably, the nucleic acid may be provided in isolated, purified or substantially purified form.

The nucleic acids of the invention comprise a sequence of DNA or RNA, including one having an open reading frame that encodes the protein of the invention and is capable, under appropriate conditions, of being expressed as one of the proteins of the invention. The term “nucleic acid” also encompasses genomic DNA, cDNA, mRNA, splice variants, antisense RNA, RNAi, DNA comprising one or more single-nucleotide polymorphisms (SNPs), and vectors comprising the subject nucleic acid sequences. Also encompassed in this term are nucleic acids that are homologous or substantially similar or identical to the nucleic acids encoding the proteins of the invention. Thus, the subject invention provides genes encoding a subject protein, and homologs thereof.

Nucleic acids or polynucleotides of the invention refer to polymeric forms of nucleotides of any length. The nucleic acids or polynucleotides can contain deoxyribonucleotides, ribonucleotides, and/or their analogs or derivatives. For example, nucleic acids can be naturally occurring DNA or RNA, or can be synthetic analogs, as known in the art. Polynucleotides of the invention also encompass genomic DNA, genes, gene fragments, exons, introns, regulatory sequences, or regulatory elements, such as promoters, enhancers, initiation and termination regions, other control regions, expression regulatory factors, and expression controls; DNA comprising one or more single-nucleotide polymorphisms (SNPs), allelic variants, isolated DNA of any sequence, and cDNA; mRNA, tRNA, rRNA, ribozymes, splice variants, antisense RNA, antisense conjugates, RNAi, and isolated RNA of any sequence; recombinant polynucleotides, heterologous polynucleotides, branched polynucleotides, labelled polynucleotides, hybrid DNA/RNA, polynucleotide constructs, vectors comprising the subject nucleic acids, nucleic acid probes, primers, and primer pairs.

Nucleic acids of the invention encompass modified nucleic acid molecules, with alterations in the backbone, sugars, or heterocyclic bases, such as methylated nucleic acid molecules, peptide nucleic acids, and nucleic acid molecule analogs, which may be suitable as, for example, probes if they demonstrate superior stability and/or binding affinity under assay conditions. They also encompass single-stranded, double-stranded, and triple helical molecules that are either DNA, RNA, or hybrid DNA/RNA and that may encode a full-length gene or a biologically active fragment thereof.

Nucleic acids of the invention include single nucleotide polymorphisms. Single nucleotide polymorphisms (SNPs) occur frequently in eukaryotic genomes. The nucleotide sequence determined from one individual of a species may differ from other allelic forms present within the population. The present invention encompasses such SNPs. The subject polynucleotides include those that encode variants of the polypeptides described in the instant specification. Thus, in some embodiments, a subject nucleic acid encodes variant proteins that include insertions, deletions, or substitutions compared with the polypeptides described herein. Conservative amino acid substitutions include serine/threonine, valine/leucine/isoleucine, asparagine/histidine/glutamine, glutamic acid/aspartic acid, etc, as described herein.

Alterations of a native amino acid sequence to produce mutant proteins, such as by insertion, deletion and/or substitution, can be done by a variety of means known to those skilled in the art. For instance, site-specific mutations can be introduced by ligating into an expression vector a synthesized oligonucleotide comprising the modified site. Oligonucleotide-directed site-specific mutagenesis procedures can also be used, such as disclosed in Walder et al., Gene 42: 133 (1986); Bauer et al., Gene 37: 73 (1985); Craik, Biotechniques, 12-19 (January 1995); and U.S. Pat. Nos. 4,518,584 and 4,737,462.

The present invention also provides a vector or expression construct comprising the nucleic acid of the invention as described herein.

The present invention also provides a cell comprising the nucleic acid of the invention or the vector or expression construct of the invention as described herein. Suitably, the cell may be an isolated cell.

### Methods of Production

The present invention also provides a method of preparing a recombinant protein of the invention comprising incubating the cell of the invention as described herein under conditions that allow the cell to express the recombinant protein.

The nucleic acid may, for example, be inserted into a suitable vector or expression construct for production of a recombinant protein by insertion of the vector or expression construct for into a prokaryotic or eukaryotic host cell. Successful expression of the recombinant protein requires that the expression vector contains the necessary regulatory elements for transcription and translation which are compatible with, and recognised by the particular host cell system used for expression. A variety of host cell systems may be utilized to express the recombinant protein, which include, but are not limited to bacteria transformed with a bacteriophage vector, plasmid vector, or cosmid DNA; yeast containing yeast vectors; fungi containing fungal vectors; insect cell lines infected with virus (e.g. baculovirus); and mammalian cell lines transfected with plasmid or viral expression vectors, or infected with recombinant virus (e.g. vaccinia virus, adenovirus, adeno-associated virus, retrovirus, etc).

Using methods known in the art of molecular biology, various promoters and enhancers can be incorporated into the expression vector, to increase the expression of the recombinant protein, provided that the increased expression of the amino acid sequences is compatible with (for example, non-toxic to) the particular host cell system used.

The selection of the promoter will depend on the expression system used. Promoters vary in strength, i.e. ability to facilitate transcription. Generally, it is desirable to use a strong promoter in order to obtain a high level of transcription of the coding nucleotide sequence and expression into recombinant protein. For example, bacterial, phage, or plasmid promoters known in the art from which a high level of transcription have been observed in a host cell system including E. coli include the lac promoter, trp promoter, recA promoter, ribosomal RNA promoter, the P_{R} and P_{L} promoters, lacUV5, ompF, bla, Ipp, and the like, may be used to provide transcription of the inserted nucleotide sequence encoding amino acid sequences.

Other control elements for efficient transcription or translation include enhancers, and regulatory signals. Enhancer sequences are DNA elements that appear to increase transcriptional efficiency in a manner relatively independent of their position and orientation with respect to a nearby coding nucleotide sequence. Thus, depending on the host cell expression vector system used, an enhancer may be placed either upstream or downstream from the inserted coding sequences to increase transcriptional efficiency. Other regulatory sites, such as transcription or translation initiation signals, can be used to regulate the expression of the coding sequence.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. In preferred embodiments, the cell is a mammalian cell, especially a HEK cell such as HEK293F cells or a CHO cell. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal, or human immunoglobulin G (IgG) signal). Additional examples of suitable signal sequences include those set out in Table 5 below.

TABLE 5

| SEQ | | Amino acid sequence |
|---|---|---|
| ID | Name | of signal peptide |
| 54 | AFS-APN | MLRLALFAVLFACAFS |
| 55 | AHA-CKC | MRVAIVFIACFAVAHA |
| 56 | SFA-QAP | MRLSVVLAVFVAGSFA |
| 57 | AQA-PNY | MRLSLVLAVFVAGNLAQAAQA |
| 58 | SQG-QSL | MIAYHFIGFVALLLPLALSQG |
| 59 | SSA-TAF | MYLWLLGIFVQLLCNAAVSSA |
| 60 | ALC-QNA | MAKFYYIVLVITALPLALC |
| 61 | CLA-KAI | MKVLFILPVVLATCLA |
| 62 | VHA-KMQ | MRSLCLLLAVVLVAVHA |
| 63 | VEA-TTR | MWGILLLLGAVVPDVLVTVEA |
| 64 | CLA-LRQ | MRQVLLLLAIVVCSCLA |
| 65 | CEG-RPV | MANLNFTALAIASVLLALCEG |
| 66 | STA-IEF | MLGSVLGIAVTACILFDVPVSTA |
| 67 | TLA-LCP | MKSSMALVALCIATTLA |
| 68 | AHS-NEH | MEAYLVVLIAIAGIAHS |
| 69 | VHG-NSM | MLVLVPLLALLAVSVHG |

In some embodiments, the signal sequences of any one of SEQ ID NOs: 54-69 is suitably used for the expression of any one of the proteins of the invention (including fragments and variants thereof). For example, the signal sequences of any one of SEQ ID NOs: 54-69 may be suitably used for the expression of an isolated, recombinant or synthetic protein comprising an amino acid sequence of any one of SEQ ID NOs:1-20 or a recombinant protein encoded by a nucleotide sequence any one of SEQ ID NOs:21-53.

In particular embodiments, one or more of the following apply:
a signal sequence of SEQ ID NO:54 is used for the expression of a protein comprising an amino acid sequence of SEQ ID NO:11;
a signal sequence of SEQ ID NO:55 is used for the expression of a protein comprising an amino acid sequence of SEQ ID NO:10;
a signal sequence of SEQ ID NO:56 is used for the expression of a protein comprising an amino acid sequence of SEQ ID NO:1;
a signal sequence of SEQ ID NO:57 is used for the expression of a protein comprising an amino acid sequence of SEQ ID NO:9;
a signal sequence of SEQ ID NO:58 is used for the expression of a protein comprising an amino acid sequence of SEQ ID NO:13;
a signal sequence of SEQ ID NO:59 is used for the expression of a protein comprising an amino acid sequence of SEQ ID NO:20;
a signal sequence of SEQ ID NO:60 is used for the expression of a protein comprising an amino acid sequence of SEQ ID NO:2;
a signal sequence of SEQ ID NO:61 is used for the expression of a protein comprising an amino acid sequence of SEQ ID NO:6;
a signal sequence of SEQ ID NO:62 is used for the expression of a protein comprising an amino acid sequence of SEQ ID NO:8;
a signal sequence of SEQ ID NO:63 is used for the expression of a protein comprising an amino acid sequence of SEQ ID NO:7;
a signal sequence of SEQ ID NO:64 is used for the expression of a protein comprising an amino acid sequence of SEQ ID NO:14;
a signal sequence of SEQ ID NO:65 is used for the expression of a protein comprising an amino acid sequence of SEQ ID NO:19;
a signal sequence of SEQ ID NO:66 is used for the expression of a protein comprising an amino acid sequence of SEQ ID NO:15;
a signal sequence of SEQ ID NO:67 is used for the expression of a protein comprising an amino acid sequence of SEQ ID NO:16;
a signal sequence of SEQ ID NO:68 is used for the expression of a protein comprising an amino acid sequence of SEQ ID NO:17;
a signal sequence of SEQ ID NO:69 is used for the expression of a protein comprising an amino acid sequence of any one of SEQ ID NOs: 1 to 20.

Exemplary promoters active in mammalian cells include cytomegalovirus immediate early promoter (CMV-IE), human elongation factor 1-αpromoter (EF1), small nuclear RNA promoters (U1a and U1b), a-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines include monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's Fl0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

The present invention also provides a method for isolating (or purifying or substantially purifying) a protein of the invention. Methods for isolating a protein are known in the art.

Where a protein of the invention is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

A protein prepared from the cells of the invention can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a polypeptide tag, e.g., a poly-histidine tad such as a hexa-histidine tag, or a myc tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

The skilled person will also be familiar with the use of appropriate tags to allow for enzymatic ligation, including but not limited to the use of the “SPYCATCHER” and/or SPYTAG sequence motifs, and the use of butelase, asparaginyl peptidase or sortase.

### Pharmaceutical Compositions

The present invention also provides a pharmaceutical composition comprising, consisting essentially of, or consisting of the protein of the invention as described herein and at least one pharmaceutically acceptable carrier.

As used herein, the term “consisting essentially of” and “consisting of” in the context of the pharmaceutical composition of the invention will be understood to imply that the composition does not comprise any additional active agents other than those specified in the composition.

Suitably, the pharmaceutical composition comprises an appropriate pharmaceutically acceptable carrier. Preferably, the pharmaceutically acceptable carrier is suitable for administration to mammals, especially humans.

As used herein, the term “pharmaceutically acceptable carrier” refers to a solid or liquid filler, diluent, excipient, solvent or encapsulating substance that may be safely used in topical or systemic administration. The carrier(s) must be “acceptable” in the sense of being compatible with the other ingredients of the composition and not deleterious to the recipient thereof.

The pharmaceutical composition may be suitably formulated for administration by a particular route. Suitable routes of administration include oral, rectal, transmucosal, transdermal, and parenteral administration. In some embodiments, the composition is formulated for oral administration, topical administration such as buccal or sublingual administration, nasal administration, intra-rectal administration, transdermal administration, or parenteral administration such as subcutaneous, intramuscular, intraperitoneal or intravenous administration. In preferred embodiments, the composition is formulated for parenteral administration, especially subcutaneous or intraperitoneal administration.

Pharmaceutical formulations include those suitable for oral, rectal, nasal, topical (including buccal and sub-lingual) or parenteral (including intramuscular, sub-cutaneous and intravenous) administration or in a form suitable for administration by inhalation or insufflation. The proteins of the invention, together with a conventional adjuvant, carrier, excipient, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. The proteins of the invention can be administered in a wide variety of parenteral dosage forms. It will be obvious to those skilled in the art that the following dosage forms may comprise, as the active component, either a protein of the invention (including a fragment or variant thereof) or a pharmaceutically acceptable derivative of the protein.

For preparing pharmaceutical compositions from a protein of the invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilisers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water-propylene glycol solutions. For example, parenteral injection liquid preparations can be formulated as solutions in aqueous polyethylene glycol solution.

The proteins according to the invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis, the proteins according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomizing spray pump. To improve nasal delivery and retention, the proteins according to the invention may be encapsulated with cyclodextrins, or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively, the active ingredient may be provided in the form of a dry powder, for example a powder mix of the active compound in a suitable powder base such as lactose, starch, starch derivatives such as hydroxypropylmethyl cellulose and polyvinylpyrrolidone (PVP).

Conveniently, the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the active compound will generally have a small particle size for example of the order of 1 to 10 microns or less. Such a particle size may be obtained by means known in the art, for example by micronisation.

When desired, formulations adapted to give sustained release of the active ingredient may be employed.

The pharmaceutical preparations can be in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The pharmaceutical compositions may further comprise an additional active agent other than the proteins of the invention. Examples of suitable additional active agents include agents useful in the treatment of an inflammatory condition (anti-inflammatory agents) and agents useful in the treatment of an autoimmune condition (immunosuppressants).

The present invention also provides kits comprising the protein of the invention as described herein, or a biologically active fragment or variant thereof, or the pharmaceutical composition described herein, and an additional active agent. In some embodiments, the additional active agent is selected from an agent useful in the treatment of an inflammatory condition and an agent useful in the treatment of an autoimmune condition.

### Methods of Use

The proteins of the invention as described herein may be useful in the treatment of an inflammatory condition or an autoimmune condition.

Accordingly, the present invention provides a method for treating or preventing an inflammatory or autoimmune condition comprising administering to a subject in need thereof the protein of the invention as described herein, or a biologically active fragment or variant thereof, or the pharmaceutical composition described herein, thereby treating or preventing an inflammatory or autoimmune condition in the subject.

The present invention also provides a method for the treatment or prevention of an inflammatory or autoimmune condition in a subject comprising the steps of:
identifying a subject having an inflammatory or autoimmune condition; and
administering to the subject in need thereof a therapeutically effective amount of the protein of the invention as described herein, or a biologically active fragment or variant thereof, or the pharmaceutical composition described herein,
   thereby treating or preventing an inflammatory or autoimmune condition in the subject.

The present invention also provides the use of the protein described herein, or a biologically active fragment or variant thereof, in the manufacture of a medicament for treating or preventing an inflammatory or autoimmune condition.

The present invention also provides the protein described herein, or a biologically active fragment or variant thereof, for use in treating or preventing an inflammatory or autoimmune condition.

The methods and uses of the invention may further comprise administering an additional active agent other than the protein of the invention. Examples of suitable active agents include agents useful in the treatment of an inflammatory condition (anti-inflammatory agents) and agents useful in the treatment of an autoimmune condition (immunosuppressants).

The additional active agent and the protein of the invention (including a fragment or variant thereof) may be administered together in a single composition or in separate compositions. Accordingly, in some embodiments, the additional active agent and the protein of the invention are administered in a single composition, such as the pharmaceutical composition described herein. In other embodiments, the additional active agent and the protein of the invention are administered simultaneously or sequentially in separate compositions. The additional active agent and the protein of the invention may be administered as different times and at different frequencies, but in combination they exert biological effects at the same time or at overlapping times.

As used herein, term “inflammatory condition” refers to a condition, disorder or disease characterised by inflammation. Symptoms of inflammation may include redness, swelling, heat, pain and loss of function. Examples of inflammatory conditions include inflammatory bowel diseases (IBDs) such as ulcerative colitis and Crohn's disease, irritable bowel syndrome (IBS), type 2 diabetes and asthma.

As used herein, the term “autoimmune condition” refers to a condition, disorder or disease in which the body's immune system mistakenly attacks the body's own cells resulting in an abnormal immune response. Autoimmune conditions may be genetic and/or caused by environmental factors such as infection and chemicals including drugs. Examples of autoimmune conditions include rheumatoid arthritis, lupus, allergy including food allergy, and an autoimmune disease of the gastrointestinal system including coeliac disease. It will be appreciated that an autoimmune condition may also be an inflammatory condition.

As used herein, the term “treating” or “treatment” of a subject include the administration of a protein of the invention (or fragment or variant thereof) or a composition described herein to a subject with the purpose of delaying, slowing, stabilizing, curing, healing, alleviating, relieving, altering, remedying, less worsening, ameliorating, improving, or affecting the inflammation associated with the disease or condition, or the symptom of the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury.

As used herein, the terms “preventing” or “prevention” are intended to refer to at least the reduction of likelihood of the risk of (or susceptibility to) acquiring a condition, disorder or disease (i.e., causing at least one of the clinical symptoms of the disease not to develop in a patient that may be exposed to or predisposed to the disease but does not yet experience or display symptoms of the disease.

A subject in need of treatment of an inflammatory condition may present a number of symptoms depending on the type of condition, disorder or disease that the inflammation is associated with. In some embodiments, a subject in need of treatment may exhibit symptoms including pain, redness, swelling, fatigue, fever, rashes, chest pain, and abdominal pain.

The existence of, improvement in, or treatment or prevention of inflammation may be determined by any clinically or biochemically relevant method as described herein or known in the art. A relevant method may be measurement of symptoms including pain, redness, swelling, fatigue, fever, rashes, chest pain, and abdominal pain. The improvement, treatment or prevention may be determined directly from the subject, or a sample or biopsy therefrom. The sample or biopsy may be of the inflamed or diseased tissue. In this case, levels of pro-inflammatory cytokines and/or anti-inflammatory cytokines may be measured before and after treatment as an indicator of treatment success. Presence of inflammatory leukocytes may be yet another indicator. In some embodiments, it will be understood that decreased levels of one or more of IFN-γ, TNF-α, IL-17A compared to untreated tissue would be an indicator of decreased inflammation or an anti-inflammatory response.

A subject in need of treatment of an autoimmune condition may present a number of symptoms depending on the type of condition, disorder or disease that the inflammation is associated with. Symptoms vary for affected location, disease causing agents and individuals. Symptoms of early autoimmune disease include fatigue, fever, malaise, joint pain and rash. Autoimmune diseases are typically diagnosed using a combination of clinical history, blood tests (to assess autoantibodies, inflammation, organ function) and other investigations such as x-rays and biopsy of affected tissues.

The existence of, improvement in, or treatment or prevention of an autoimmune may be determined by any clinically or biochemically relevant method as described herein or known in the art. A relevant method may be measurement of symptoms including inflammation, fatigue, fever, rashes and pain. The improvement, treatment or prevention may be determined directly from the subject, or a sample or biopsy therefrom. The sample or biopsy may be of the affected tissue. In this case, levels of immune cells and molecule (e.g. antigens) may be measured before and after treatment as an indicator of treatment success. Presence of pro-inflammatory cytokines, anti-inflammatory cytokines and/or inflammatory leukocytes may be yet another indicator. In some embodiments, it will be understood that decreased levels of autoantibodies compared to untreated tissue would be an indicator of a decreased immune response.

In some embodiments, a method of the invention comprises administering a therapeutically or prophylactically effective amount of the protein of the invention as described herein or the pharmaceutical composition as described herein.

As used herein, the term “therapeutically effective amount” is generally intended to refer to an amount of an active agent, such as a protein of the invention (or fragment or variant thereof), that (i) treats the particular condition, disorder or disease, (ii) attenuates, ameliorates or eliminates one or more symptoms of the particular condition, disorder or disease, or (iii) delays the onset of one or more symptoms of the particular condition, disorder or disease as described herein. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

As used herein, the term “prophylactically effective amount” is intended to refer to a sufficient quantity of a protein of the invention (or fragment or variant thereof) to prevent or inhibit or delay the onset of one or more detectable symptoms of a clinical condition. Those skilled in the art will be aware that such an amount will vary depending on, for example, the particular subject and/or the type or severity or level of condition and/or predisposition (genetic or otherwise) to the condition. Accordingly, this term is not to be construed to limit the invention to a specific quantity, e.g., weight of the peptide, rather the invention encompasses any amount of the protein of the invention sufficient to achieve the stated result in a subject.

Suitable dosages of a protein of the invention will vary depending on the specific the condition to be treated and/or the subject being treated. It is within the ability of a skilled physician to determine a suitable dosage, for example by commencing with a sub-optimal dosage and incrementally modifying the dosage to determine an optimal or useful dosage. Alternatively, to determine an appropriate dosage for treatment/prophylaxis, data from cell culture assays or animal studies may be used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

Typically, a therapeutically effective dosage is formulated to contain a concentration (by weight) of at least about 0.1% up to about 50% or more of the protein of the invention, and all combinations and sub-combinations of ranges therein. The pharmaceutical composition as described herein can be formulated to contain a protein of the invention or a biologically active fragment or variant thereof a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 1 6, 1 5, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue in the subject.

The protein of the invention as described herein or the pharmaceutical composition as described herein may be administered, or formulated for administration by, any route described herein. As used herein, the term “administered” means administration of a therapeutically effective dose of the protein of the invention to the subject. As used herein, the term “formulated for administration” means a therapeutically effective dose of the protein of the invention is formulated in such a way that is suitable for the route of administration. In preferred embodiments, the protein of the invention (or pharmaceutical composition) is administered parenterally. In other preferred embodiments, the protein of the invention (or pharmaceutical composition) is formulated for parenteral administration.

The frequency of administration of a protein of the invention or pharmaceutical composition as described herein may be once daily, twice daily or three times daily. The treatment period may be for the duration of the detectable disease.

In some embodiments, the inflammatory or autoimmune condition is selected from an inflammatory bowel disease, irritable bowel syndrome, type 2 diabetes, asthma, rheumatoid arthritis, lupus, allergy, and coeliac disease, especially an inflammatory bowel disease. In embodiments where the inflammatory condition is an inflammatory bowel disease, the inflammatory bowel disease may be ulcerative colitis or Crohn's disease.

Inflammatory bowel disease (IBD) is a group of chronic inflammatory disorders of the digestive tract. It can develop as ulcerative colitis causing long-lasting inflammation and ulcers in the lining of large intestine and rectum, or as Crohn's disease characterized by inflammation of the lining of digestive tract dispersing into affected tissues such as mouth, esophagus, stomach and the anus.

Crohn's disease is a condition characterised by chronic inflammation in the lining of the digestive system. There may be a small patch of inflammation, or it may spread quite a way along the gut, or there may be several patches in different places. Typical symptoms include recurring diarrhoea, often with a feeling of urgency to get to the toilet, and often with a feeling of wanting to go to the toilet but with nothing to pass, abdominal pain and cramping, which is usually worse after eating, extreme tiredness (fatigue) and/or weight loss. Colonoscopy is used for diagnosis via sampling of small tissue samples (biopsies) for examination under the microscope.

Ulcerative colitis is a chronic inflammatory condition that usually occurs in the rectum (the part of the large bowel that lies just inside the anus) and lower part of the colon, but it may affect the entire large intestine (colon). The colon becomes inflamed and, if this inflammation becomes severe, the lining of the colon is breached and ulcers may form. Diagnosis of ulcerative colitis may be performed via blood test, to check for inflammation, anaemia and protein levels, stool sample, which is checked for infection, X-rays, to help assess the extent of the condition, sigmoidoscopy, to examine the extent of inflammation in the rectum and lower part of the colon and/or colonoscopy, to examine the inside of the entire colon.

Irritable bowel syndrome (IBS) is a gastrointestinal disorder characterised by a group of symptoms experienced together including abdominal pain and alternating diarrhoea and constipation. Diagnosis of IBS may be performed via blood test (including blood tests for coeliac disease), stool test, and investigation of the bowel (eg sigmoidoscopy or colonoscopy).

Type 2 diabetes is a progressive condition in which the body becomes resistant to the normal effects of insulin and/or gradually loses the capacity to produce enough insulin in the pancreas. Type 2 diabetes is typically characterised by high blood glucose in the context of insulin resistance and reduced insulin production. Symptoms of type 2 diabetes may include excessive thirst, increased hunger, frequent urination, feeling lethargic, and poor wound healing. Diagnosis of type 2 diabetes may be performed via blood glucose testing (fasting or random).

Asthma is a condition in which the airway becomes inflamed, narrow and swell and produce extra mucus. This may make breathing difficult and cause chest pain, coughing and wheezing. Diagnosis may be performed by monitoring the pattern of symptoms and response to therapy over time, and spirometry to determine the volume of air that can forcibly be blown out in first 1 second, after full inspiration (FEV1).

Rheumatoid arthritis (RA) is a chronic autoimmune and inflammatory condition that primarily affects joints, typically causing pain and swelling of the joints. In RA, the immune system targets the lining of the joints, causing inflammation and joint damage. Diagnosis may be performed by monitoring symptoms and from various test including blood tests for detecting inflammation and antibodies such as anti-cyclic citrullinated peptide (anti-CCP) and x-rays.

Lupus is a chronic autoimmune condition which can cause inflammation in many different parts of the body. Systemic lupus erythematosus (SLE) is a type of lupus that can affect almost any organ or system of the body. Common symptoms may include joint and muscle pain, skin rashes, fever, and fatigue. Diagnosis may be performed via blood test, typically including an antinuclear antibody (ANA) test.

Allergies are conditions caused by hypersensitivity of the immune system to a trigger or allergen. Examples of allergic conditions include hay fever, food allergies, atopic dermatitis, hives and allergic asthma. Symptoms or allergic reactions may include itchiness, sneezing, difficulty breathing, and anaphylaxis. Diagnosis may be performing by allergy tests, including skin prick tests, patch tests, challenge tests, and blood tests.

Coeliac disease is a chronic autoimmune condition that primarily affects the small intestine and is caused by a reaction to gluten. Coeliac disease usually occurs in people who are genetically predisposed. Symptoms typically include gastrointestinal problems including chronic diarrhoea, abdominal distention, malabsorption, loss of appetite, and among children failure to grow normally. Diagnosis may be performed by a combination of tests including blood test for detecting antibodies, intestinal biopsies and genetic testing.

Although the peptide of the invention finds application in humans, it will be understood that the invention is also useful for veterinary purposes. Thus in all aspects the invention is useful for domestic animals such as cattle, sheep, horses and poultry; for companion animals such as cats and dogs; and for zoo animals. Therefore, the general term “subject” or “subject to be/being treated” will be understood to include all animals (such as humans, apes, dogs, cats, horses, and cows) that may have an inflammatory condition.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1. Selection of Protein-Coding Genes

The proteins of SEQ ID NOs:1-20 were selected from protein-coding genes identified from two dataset inputs, namely Adult ES Proteome dataset input Mulvenna et al (Proteomic analysis of the excretory/secretory component of the blood-feeding stage of the hookworm, Ancylostoma caninum. Mol Cell Proteomics. 2009;8:109-21) and Activated L3 SSH Transcriptome databased input Datu et al (Transcriptional Changes in the Hookworm, Ancylostoma caninum, during the Transition from a Free-Living to a Parasitic Larva. PLoS Negl Trop Dis. 2008:2(1):e130), using the following bioinformatic workflow.
Step 1—Compiling gene lists from input datasets: Protein-coding genes identified in dataset 1 (Adult ES Proteome-Mulvenna et al) were based on their predicted Gene Ontology (GO) from the NCBI database (2007).
Protein-coding genes were identified at the mRNA level in dataset 2 (Activated L3 SSH Transcriptome-Datu et al) based on upregulation at the mRNA level in serum-activated L3 (Datu et al, Transcriptional Changes in the Hookworm, Ancylostoma caninum, during the Transition from a Free-Living to a Parasitic Larva. PLoS Negl Trop Dis. 2008:2(1):e130). The most highly expressed mRNAs were prioritised according to the Log2 signal intensity detected by SSH. The selection criteria excluded all mRNAs that were down-regulated upon serum stimulation. The NCBI reference database has been vastly improved and expanded due to advances in deep sequencing technologies and bioinformatic analyses since the Mulvenna et al and Datu et al studies were conducted. In these studies, protein-coding genes were predicted using bioinformatics software compared each read to gene models from other parasitic and free-living nematodes such as Brugia malayi and Caenorhabditis elegans, respectively (https://parasite.wormbase.org/Brugia_malayi_prjna10729/Info/Index/), (https://www.pacb.com/auto_tags/c-elegans/).
After the above initial studies were conducted, more than 1.5 million expressed sequence tags (ESTs; sequences derived from randomly selected cDNA clones) had been subsequently generated from different developmental stages of A. caninum (Wang Z et al, Characterizing Ancylostoma caninum transcriptome and exploring nematode parasitic adaptation. BMC Genomics. 2010 May 14;11:307). Therefore, subsequent blast comparisons of the selected ESTs were conducted against the up-to-date databases and to yield more accurate predicted gene ontologies. One hundred and seventy-four protein-coding genes were selected in step 1.
Step 2—Establishing whether the selected ESTs are full-length sequences: The NCBI BLASTx tool, using default parameters of the nucleotide sequence database (nr/nt) was used to establish whether the 174 selected ESTs were indeed full-length (https://blast.ncbi.nlm.nih.gov/Blast.cgi?PAGE_TYPE=BlastSearch). The individual translated sequence queries were searched against the up-to-date protein databases to ascertain if sequences were full-length by comparison to the top BLAST hits. It was evident that some of the assembled nucleotide sequences appeared to be truncated at the 5′ end or there was an observed reading frame shift and therefore the full-length protein-coding gene was not present; as a result, some of these proteins were flagged as partial-length EST sequences.
Step 3—Establishing whether the EST sequences have a defined signal peptide: Each EST sequence was queried for the presence of a defined N-terminal signal peptide (SP) by comparison to homologous sequences in NCBI (2016). SPs direct a protein to be transported through the secretory pathway and outside the cell. However, in cell-free recombinant protein engineering SPs are redundant because there is no facility for directing sequences through the secretory pathway. Furthermore, SPs are hydrophobic to facilitate translocation through membranes of the various organelles and the cell itself, and this is an undesirable attribute in recombinant protein expression where soluble proteins are required. The presence, location and cleavage site of an N-terminal SP was predicted for each query EST using SignalP 4.1 Server with default parameters (http://www.cbs.dtu.dk/services/SignalP/). Query EST sequences with close homologues that contained SPs proceeded to step 9. Any query EST sequence without an obvious SP proceeded to step 4.
Step 4—Mapping partial-length transcripts: The partial-length transcripts (without a defined start or stop codon or with an ambiguous SP status) were mapped to PacBio lsoseq sequencing Reads of Insert (Rol). This data was acquired through a collaboration with Dr Makedonka Mitreva at Washington University School of Medicine in St. Louis, U.S.A. PacBio IsoSeq sequencing technology generates long sequencing reads that are up to 10 kb with low systematic bias and a high consensus accuracy (https://www.pacb.com/products-and-services/). Pacific Biosciences long ead sequencing technology (PacBio) reads that mapped to the partial-length A. caninum ESTs were extracted. Query ESTs were mapped against the A. caninum reference assembly and gene models using HISAT2 (https://bio.tools/HISAT2). The transcript consensus sequences are available at http://nematode.net. High quality PacBio reads were extracted from A. caninum transcript data and were mapped to the same reference. Lists of the A. caninum PacBio Rol that mapped to those genes identified by the ESTs were built and the FASTQ data was extracted.
Steps 5 and 6—Obtaining reads: PacBio reads for 11 of the 18 protein-coding genes of interest were obtained. The A. caninum reference genome assembly was obtained from WormBase ParaSite (version: WBPS11-WS265-A_caninum_9.3.2.ec.cg.pg) and contained 25,339 contigs (step 6 of the bioinformatic workflow).
Steps 7 and 8—Aligning reads to reference genome and obtaining full-length protein-coding sequences: The PacBio reads were aligned to the reference genome using STAR transcriptome aligner (STAR_2.5.2b) with default parameters (https://github.com/alexdobin/STAR). The predicted gene model was obtained from WormBase ParaSite (version: WBPS11-WS265) which contained 30,198 predicted transcripts (https://parasite.wormbase.org/Ancylostoma_caninum_prjna72585/Info/Index/). Integrative Genomic Viewer (IGV) software (version 2.3.72) was used to view the alignments and further translate the PacBio reads into all 6 reading frames, which were subsequently compared to the predicted WormBase ParaSite gene model alignments (https://software.broadinstitute.org/software/igv/Rel Notes2.3.x).
In more than 50% of cases, the PacBio read transcript sequence did not match the gene model. Discrepancies detected between the WormBase ParaSite predicted gene model and the actual PacBio read transcripts included the detection of truncated proteins that were shorter than the transcript, small changes in exon boundaries, skipped exons and new exons.
After verification of the location of the start codon, SignalP online software was employed to identify whether the start codon was followed by a SP. If the start codon was not followed by an SP, then all six frames were examined to deduce if there was a change in the ORF to locate an SP. The location of the stop codon was determined and untranslated regions (UTRs) were also excluded. The final full-length protein-coding sequences were taken forward to step 9
Step 9—Generating open reading frames for cloning: Any identified SP and stop codons were removed from the corresponding nucleotide sequences to generate ORFs for cloning into the plasmid vector for cell-free protein expression.

### Example 2. Cell-Free Lysate Protein Production in the Leishmania tarentolae Extract (LTE) System

The selected protein-coding ORFs were synthesised and ligated into the pLTE vector by Protein CT Biotechnologies. The pLTE DNA was propagated and cell-free lysate protein expression was optimised at James Cook University (JCU). eGFP-fused lysate protein expression was monitored in relative fluorescence units (RFU) overtime on a POLARstar® Omega Plate Reader Spectrophotometer. Detection of eGFP-expression from a selection of the 101 LTE fusion proteins. Expression was measured in relative fluorescence units (RFU) over time of the reaction in minutes a wavelength range of 485 nm excitation and 520 nm emission. Protein expression was further validated by SDS-PAGE electrophoresis of the non-denatured cell-free lysate. Fluoresceinated proteins were visualised using the 485 nm excitation to 520 nm emission filter on a BIO-RAD™ VersaDoc Imaging System.

### Example 3. Administration of Non-Recombinant Cell-Free Lysate to Mice

To investigate whether recombinant cell-free lysates could be used in vivo without the need to purify the hookworm recombinant proteins, mice with trinitrobenzoic acid (TNBS)-induced colitis were administered either positive or negative non-recombinant lysate by intraperitoneal (i.p.) injection. There was no apparent effect of i.p. administration of the positive or negative non-recombinant lysates on the measured parameters of TNBS-induced colitis in mice. This result strengthened the rationale for using the TNBS-induced colitis model to test cell-free lysates containing hookworm recombinant proteins in vivo.

### Example 4. Effect of Recombinant Protein Lysates in TNBS-Induced Colitis Model

The recombinant protein lysates were assessed in a mouse model of TNBS-induced colitis directly, without prior assessment of the in vitro biological activity of the lysates. Naïve mice received DPBS only, control mice received non-recombinant cell-free lysate followed by TNBS and the remaining experimental groups received hookworm recombinant protein lysate followed by TNBS. After the induction of colitis by TNBS, the mice developed various manifestations of acute colitis such as inconsistent stool formation, bloody diarrhoea and a dramatic loss of body weight.

### Procedure

Leishmania tarentolae Expression (LTE) Protocol

Hookworm cDNA open reading frames (ORFs) were cloned into the plasmid vector, pLTE eGFP. Protein-coding ORF sequences were sent to Protein CT Biotechnologies and genes were synthesised. Sufficient lysate (600 μL) for each recombinant protein was prepared to enable intraperitoneal (IP) injection of 100 μL LTE lysate to each of 5 mice. Lysate reactions were conducted in RNase/DNase-free 96-well culture plates. Four hundred and twenty (420) μL of lysate was added to 1 pg DNA (50 μg/mL), 1.5 μL RNAse OUT ribonuclease inhibitor (final concentration 0.25%) and topped up to a final volume of 600 μL with MilliQ H20. The relative fluorescence units (RFU) produced by translation of eGFP-fused protein was continuously monitored for 2 hours on a POLARstar Omega Plate Reader Spectrophotometer (BMG LABTECH) with a wavelength range of 485 nm excitation and 520 nm emission. The lysate reaction was centrifuged for 1 minute at 300 g and the pellet was discarded. Ten (10) μL of the supernatant was loaded onto a 12% SDS-PAGE gel in an XCell SureLocka Mini-Cell Electrophoresis System (ThermoFisher Scientific) and subjected to 140 volts for 30 minutes using a PowerPac Basic Power Supply (BIO-RAD). The fluorescence signal emitted by the eGFP-tagged recombinant protein band was visualised using a BIO-RAD VersaDoc Imaging System with the wavelength range of 485 nm excitation and 520 nm emission.

The following lysates were evaluated:
Lysate 2, containing SEQ ID NO:6 described herein.
Lysate 9, containing SEQ ID NO:15 described herein.
Lysate 11, containing SEQ ID NO:17 described herein.
Lysate 13, containing SEQ ID NO:1 described herein.
Lysate 15, containing SEQ ID NO:13 described herein.
Lysate 16, containing SEQ ID NO:2 described herein.
Lysate 17, containing SEQ ID NO:19 described herein.
Lysate 19, containing SEQ ID NO:9 described herein.

The following lysates were also evaluated for comparison:
Lysate 10, containing SEQ ID NO:70 shown in Table 6.
Lysate 12, containing SEQ ID NO:71 shown in Table 6.
Lysate 14, containing SEQ ID NO:72 shown in Table 6.
Lysate 20, containing SEQ ID NO:73 shown in Table 6.

TABLE 6

| SEQ | | |
|---|---|---|
| ID | Lysate | Amino acid sequence of protein |
| 70 | 10 | MRDDFLDEHNMRRSDVAEGAIPLGPRGKLCPTAARMPKL |
| | | SYDCNLEKNAYEFARQCSLGGSSESSRQDQGENFHVAPY |
| | | NGSPTEVGIRAINEWFNEITRKGMNRQMNFTPGIPLVKP |
| | | KLTSFTQ |
| 71 | 12 | TDFQCWNFKSTDTLREHYLKSINNLRKKIADGSAENKSG |
| | | KCPQGKNIYKLSWDCELELKAQQAVDQCKPNVPEPAGYS |
| | | QILKKVKSTCDPTKVLKKQIEAWWTKSVKDAGVDNPPNN |
| | | KQGLEDFAKLANGKATKIGCAQKNCNEQLYVACVINEPA |
| | | PAVGMPIYEVGAGCNSKDDCTTYLQSKCSNKVCVAGHPG |
| | | DATTTTSTPATTAPTTPTIPAGPTTAPAPPPTTAAPTTT |
| | | STIGSIDNTICPQNQVITDSVRLTFLNTHNGLRSQLAQG |
| | | QIFMGNGARARPASKMRRMVYNCDAESSARNSAAQCLSS |
| | | PGSPSGYTENLHVINNNFVDHNSAATQAFNAWWSEINTG |
| | | YMRQAETERNMYSLSVGIPNFAKMAWETNAHLGCAIVRC |
| | | GLNTNVVCPYSPKSDGGQIYKMGPFCRRCPDYPGTFCNQ |
| | | GLCSF |
| 72 | 14 | LEIVVDNQGSGCSVDRQYREAIDKFHNELRQRIARGDAE |
| | | KPGQQMYGLVYDCGLEKEADYETRLPGYADLHHHGVTRF |
| | | SGDYERSALSALREMLKTFSTDKNAMRQVTYPKATRFGC |
| | | TARLRRNKKTGMRRMDWVCVYDKKPKDGESFEGGKPCNE |
| | | NKDCTYYKGSTCEWNLCYTFF |
| 73 | 20 | MEFVCLYDKKPQDGESLEGGSYCKVDQDCTFYKRSYCED |
| | | NLCYVQ |

### Animal Procedures

All animal experiments were conducted under the parameters stipulated in the JCU Animal Ethics Committee approved project #A2180. Male BALB/c mice aged between 5 and 6 weeks old were purchased from the Animal Resource Centre (ARC) in Perth, Western Australia. Mice were weight-matched to within 1 g of each other prior to shipping to Cairns. Mice were housed in accordance with JCU animal rights and regulations under specific pathogen-free conditions (Cairns Campus). All procedures were performed after appropriate training was undertaken on the relevant handling procedures, and procedural records were kept by the manager of the facility. Mice were assessed for health status and body weight upon arrival to the facility. Based on their weight, mice were equally distributed into groups (n=5 mice per group) and housed in plastic cages with unlimited access to food and water. Mice were rested for seven days prior to the commencement of an experiment. In each experiment, each group of five mice received an injection of cell-free lysate containing a distinct hookworm recombinant protein. Each experiment had a naïve group that was administered with 100 μl DPBS (Gibco 14190144) via the intra-peritoneal (i.p.) route, a negative control group that received cell-free lysate containing empty pLTE vector expressing GFP alone, and 5 to 10 groups each of which received a different hookworm recombinant lysate. One hundred (100) μL of lysate was administered to each animal within an experimental group via IP injection 20 hours prior to the intra-rectal (IR) administration of TNBS.

### Group Treatments:

1. Naïve (DPBS only) n=5
2. TNBS+non-recombinant negative control (empty pLTE vector) n=5
3+. TNBS+Experimental groups (Lysate with hookworm recombinant protein) n=5

### Preparation of Anaesthetic

Anaesthetic was prepared aseptically to a concentration of 6.25% ketamine (as hydrochloride) (Ketamil; Provet) (50 mg/kg) and 6.25% xylazine (2-2,6 Xylidine-5,6 dihydro-4H-1,3-thiazine hydrochloride) (Xylazil; Provet) (5 mg/kg) in sterile phosphate buffered saline (DPBS) (Gibco) solution and administered to mice at a dose of 200 μL per mouse. Anaesthetic drugs were prepared on an ad hoc basis and stored at 4° C. until use under DS4-DS8 approval from the Queensland Government. A copy of the approval is held by JCU Australian Institute of Tropical Health and Medicine. Anaesthetic drugs were held in a secure location and all usage was recorded in drug usage logs.

### Administration of TNBS to Mice

In a laminar flow cabinet, mice were administered a 200 μL i.p. injection of anaesthetic solution with a 29-gauge needle in the lower right quadrant of the peritoneum. During this procedure, animals were lightly restrained with the use of a scruff pad. After sedation, when each mouse was unresponsive to stimuli, their baseline weight was recorded. For administration of the TNBS solution, mice were inverted, lubricant (Durex K-Y Jelly) was placed on the rectum and a SRPLO I.V. soft catheter (Radiopaque/ETFE, Gauge 20 G×11/4″, I.D. 0.80×32 mm) was inserted 4 mm into the colon. A volume of 100 μL of 1.5 mg 5% (w/v) 2,4,6-trinitrobenzenesulfonic acid solution (TNBS) (Sigma-Aldrich) in sterile filtered 50% ethanol in Milli-Q purified water was slowly administered into the colon and mice were held upside down for 1 minute to prevent anal leakage. Mice were returned to the appropriate cage and monitored for 30 minutes or until they regained consciousness.

### Measuring and Monitoring

Each animal was monitored daily for the three days post-TN BS administration for changes in health and physical welfare. The clinical score of each mouse was recorded daily which included body weight changes, decreased motor activity, piloerection, stool consistency and rectal bleeding.

### Clinical Score:

| Score | % Weight loss | Piloerection | Faeces | Mobility |
|---|---|---|---|---|
| 0 | Gain | None | Normal | Normal |
| 1 | Same | Mild | Mild diarrhoea | Mobile |
| 2 | Loss | Severe | Severe diarrhoea | Lethargic |

Animals exposed to TNBS treatment in the absence of therapeutic intervention were expected to cease weight loss by experimental day three. Any mouse suffering from undue distress or that lost more than one-third of its initial body weight over a 24-hour period was sacrificed based on ethical obligations in the approved protocol. In the event of this occurrence or any unexpected deaths, an “Adverse Event Form” was lodged with the JCU Animal Ethics Committee. It is widely accepted that there is no reliable positive control drug for use in TNBS colitis, and all of the standard immunosuppressive agents and biologics perform relatively poorly and inconsistently in this acute model of disease. As such, we did not include a positive control treatment in these studies, and instead relied on groups of healthy mice that did not receive TN BS and negative control mice that received empty vector lysate and were administered TNBS.

### Sacrifice and Tissue Collection

On the third day of the experiment, mice were euthanized by carbon dioxide asphyxiation and necropsied. After necropsy, the remnants of the mouse were autoclaved and disposed of via JCU approved channels. The colon was removed, measured and photographed. The macroscopic pathology score was assessed by longitudinally sectioning the colon, washing the intra-rectal contents out with sterile DPBS (Gibco) and the tissue integrity was observed under light microscopy (Olympus SX61, 0.67-45x). Macroscopic pathology score was used to assess the severity of bowel wall thickening, adhesion to internal organs and tissues, ulceration, and mucosal oedema as below.

### Macroscopic Pathology Score:

| | | | Bowel wall | Mucosal |
|---|---|---|---|---|
| Score | Adhesion | Ulceration | thickening | oedema |
| 0 | Easily removed | None | Normal thin | No oedema |
| | | | colon | |
| 1 | Minimal | Minimal (1 ulcer) | Slight | Slight |
| | resistance | | thickening | |
| 2 | Adhesions | Mild (multiple) | Noticeable | Clearly |
| | | | thickening | noticeable |
| 3 | Severe adhesion | Severe | Severe | Severe |
| | | (necrosis) | thickening | |

### Colon Sectioning

A 0.5 cm section of tissue proximal to the rectum was discarded. The next most proximal 0.5 cm section of tissue was flushed with Dulbecco's phosphate-buffered saline (DPBS) and fixed in 4% formaldehyde (Sigma-Aldrich) for a maximum of 24 hours, after which the tissue was transferred to 70% ethanol and stored at room temperature.

### Within-Experiment Statistical Analysis

Statistical analysis of different outcomes to determine overall significance of a hookworm protein lysate's performance within an experiment was conducted by undertaking statistical comparisons between each hookworm recombinant protein lysate and the nonrecombinant empty vector lysate control in each experiment using R version 3.5.1. The percentage weight change on day three and colon length were assessed using a two-sample t-test. Macroscopic and clinical scoring were assessed using a Mann-Whitney U test as this is most appropriate for ordinal data. P values were adjusted for multiple comparisons using Benjamini and Hochberg False Discovery Rate (FDR). The geometric mean of significance of these four outcomes was used to rank the protective capacity of the protein lysates.

### Statistical Analysis

In order to combine the four clinical outcomes into one value, the Z-score of each outcome for each animal was determined. The mean, standard deviation and sample size of the data set was determined and used to calculate the Z-score for each outcome of each animal. The Z-score transformation was applied to groups by subtracting the mean from each outcome, and the result was divided by the standard deviation. The signs of outcomes 3 and 4 were reversed because macroscopic scores (outcome 3) and clinical scores (outcome 4) are lower in healthy mice and elevated in diseased mice, whereas healthy mice have higher weights (outcome 1) and longer colons (outcome 2). A combined score was created by summing together the four Z-scores for each outcome. The significance of the combined Z-score of each protein lysate was determined using a Mann-Whitney one-tailed student's t-test of the combined Z-score of each animal within an experimental group compared to the negative control combined Z-scores and the data was adjusted to compensate for multiple testing.

### Results

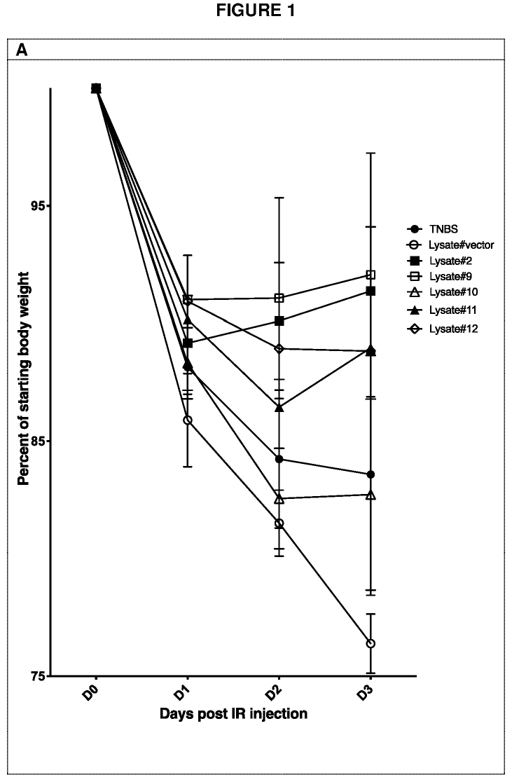

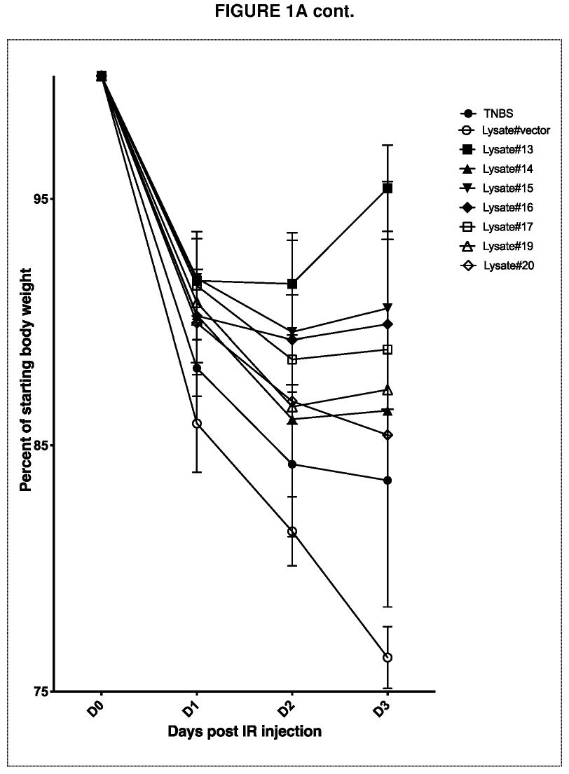

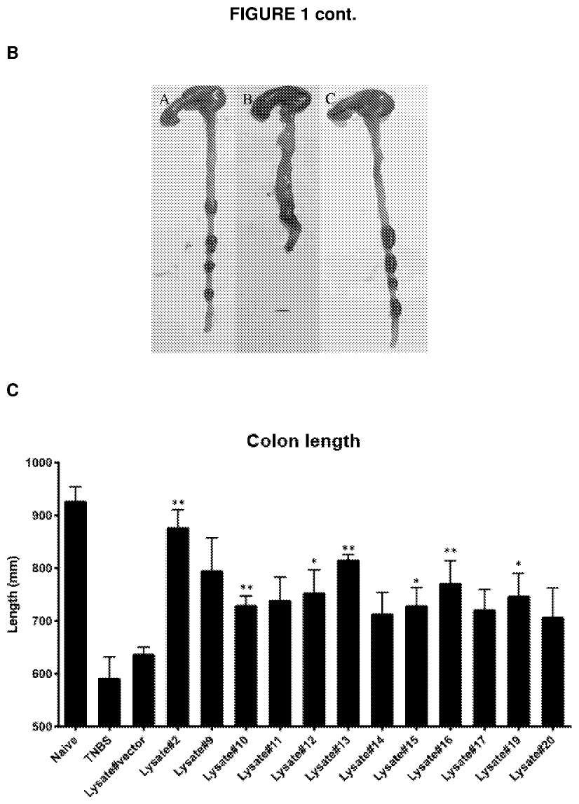

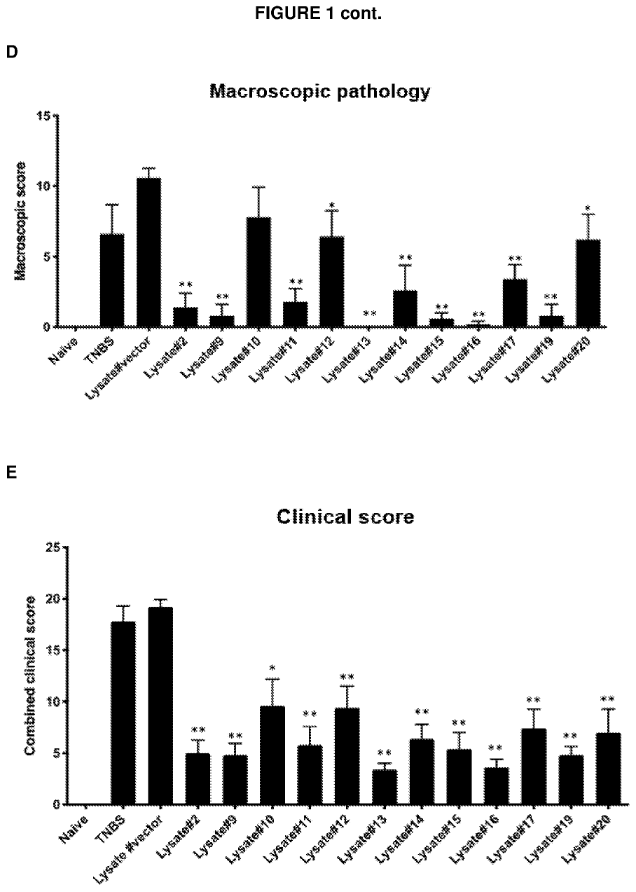

FIG. 1A shows daily weight change of mice receiving hookworm lysates before administration of TNBS. The negative control group quickly lost weight by day one post-TNBS administration and continued to do so until the termination of the experiment. Mice treated with cell-free lysates containing hookworm proteins prior to administration of TNBS displayed varying degrees of protection against weight loss. Mice treated with lysates 2, 13, 16 and 17 remained healthy and lost significantly less body weight by day 3 than negative control mice.

Upon necropsy, the colons were removed from mice for measurement and further analysis. Colon length is a good indicator of the extent of TNBS-induced colitis because in this model inflammation disseminates in a transverse manner, eventuating in transmural colitis with colon shortening (Wirtz S et al, Chemically induced mouse models of acute and chronic intestinal inflammation. Nat Protoc. 2017 July;12(7):1295-1309). FIG. 1B shows the visual differences between the colons of the naïve (A), the negative control (B) and the hookworm recombinant protein 13-containing lysate group (C). The representative colon removed from a mouse in the naïve group appears to be healthy, while the colon removed from a mouse in the negative control group was severely inflamed and shortened. The colon removed from a mouse in the group that received hookworm recombinant protein 13-containing lysate prior to TNBS was protected from TNBS-induced shortening of the colon.

FIG. 1C shows colon lengths of mice receiving hookworm protein-containing lysates prior to administration of TNBS. Mice treated with lysates 2, 10, 13 and 16 had significantly longer colons than mice in the empty vector lysate control group.

FIG. 1D shows macroscopic pathology scores of mice receiving hookworm protein-containing lysates before administration of TNBS. The colons of the negative control group scored highly due to extensive colitic inflammation, whilst groups receiving hookworm recombinant protein lysates (notably lysates 2, 9, 11, 13, 14, 15, 16, 17 and 19) maintained the integrity of the colon and their scores were significantly lower. Mice treated with lysate 2, 13 and 16 were protected against TNBS-induced weight loss as well as macroscopic pathology.

Throughout the three-day experiment, the extent of TNBS-colitis on the physical appearance of the animals was monitored and a clinical score determined for each experimental group. The clinical score incorporates non-specific physiological signs that point to an overall deterioration in the health and wellbeing of the animal including physical appearance (piloerection), faecal consistency, body weight change and behaviour (decreased movement and mobility) throughout the entirety of the experiment. A low clinical score is demonstrative of the protective properties of a hookworm recombinant protein lysate against TNBS-induced colitis.

FIG. 1E shows the combined clinical score of mice within each experimental group during the experiment. The negative control group had a significantly increased clinical score compared to the naïve group. Meanwhile, mice that received cell-free lysates 2, 13 and 6 had a clinical score comparable to the naïve group, which was in congruence with the weight loss findings. Mice treated with lysates 9, 11, 12, 14, 15, 17, 19 and 20 also had a significantly lower clinical score.

### Example 4. Bioactivity of Recombinant Proteins on Human Immune Cells

To investigate the activity of the recombinant proteins on human immune cells, human T-cells were isolated from punch biopsies obtained from ulcerative colitis patients, and stimulated with aCD3/CD28 coated beads in the presence of recombinant proteins SEQ ID NOs:11, 1, 4, 6, 8 and 9 (also referred to as AcFar2, Ac22177, Ac07727, Ac07322, Ac08034 and Ac07062, respectively; 10 μg/mL for AcFar2, 50 μg/mL for the other proteins). The recombinant proteins were expressed from HEK cells and purified. No bead stimulation was used as the negative control, and 10 μg/ml cyclosporine-A (CSA; an established T-cell inhibitor) was used the positive control. Hookworm protein Na-AlP-1, which has anti-inflammatory activity, was also used as a comparison. Na-AIP-1 was expressed in yeast and purified as per Buitrago, G. et al (A netrin domain-containing protein secreted by the human hookworm Necator americanus protects against CD4 T cell transfer coli. Transl Res. 2021 Mar. 3;S1931-5244(21)00049-9). After 72 hours the culture supernatants were collected and cytokine levels were assessed with a Legendplex (BioLegend) assay.

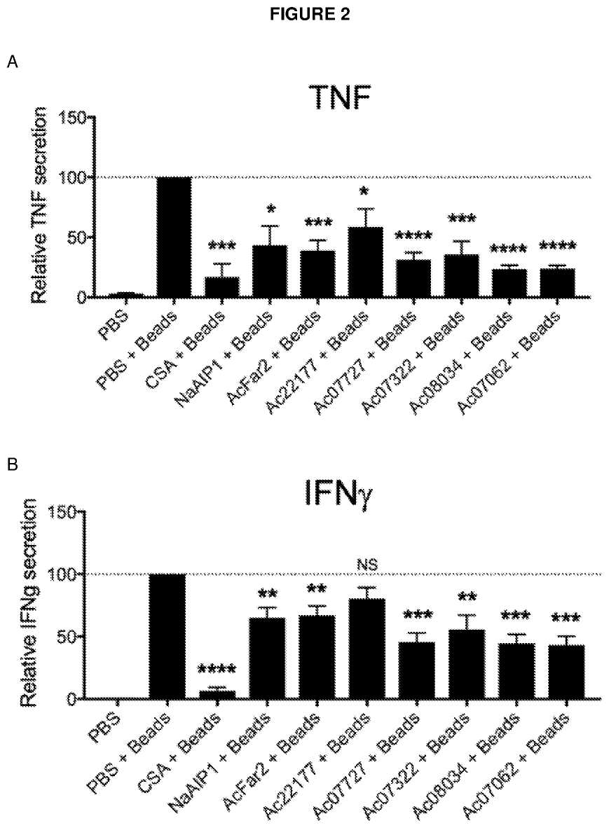

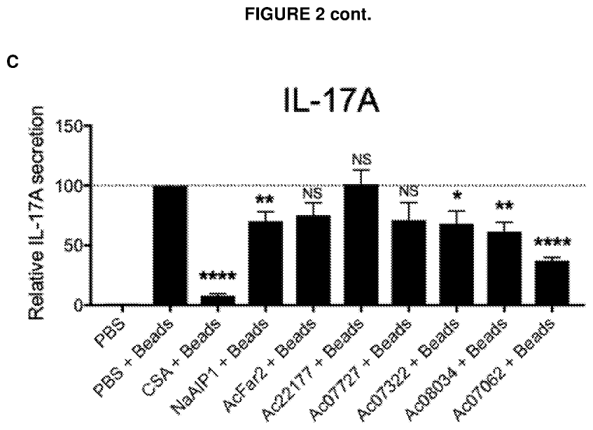

FIG. 2 shows the effect of the recombinant proteins on the secretion of inflammatory cytokines tumour necrosis factor alpha (TNFα; FIG. 2A), interferon gamma (IFNγ; FIG. 2B) and interleukin-17A (IL-17A; FIG. 2C). TNFα secretion was significantly reduced by all proteins. All proteins except for SEQ ID NO:13 (Ac2 2177) significantly reduced levels of IFNγ. Recombinant proteins SEQ ID NOs:6 (Ac07322), 8 (Ac08034) and 9 (Ac07062) significantly reduced levels of IL-17A. Collectively, these data show the recombinant proteins are effective at reducing inflammatory cytokine secretion in human T-cells.

### Example 5. Effect of Recombinant Protein Lysates in TNBS-Induced Colitis Model

Crude recombinant protein-containing lysates were assessed in a mouse model of TNBS-induced colitis directly, without prior assessment of the in vitro biological activity of the lysates. The protens were selected and expressed as described in Examples 1 and 2. Testing a single administration of the crude hookworm protein-containing lysates enabled high-throughput in vivo screening of 78 proteins within 3 days. Mice were treated with lysates containing a protein of interest, or eGFP alone as a control, via intra-peritoneal injection one day before TNBS administration, or were left untreated (naïve).

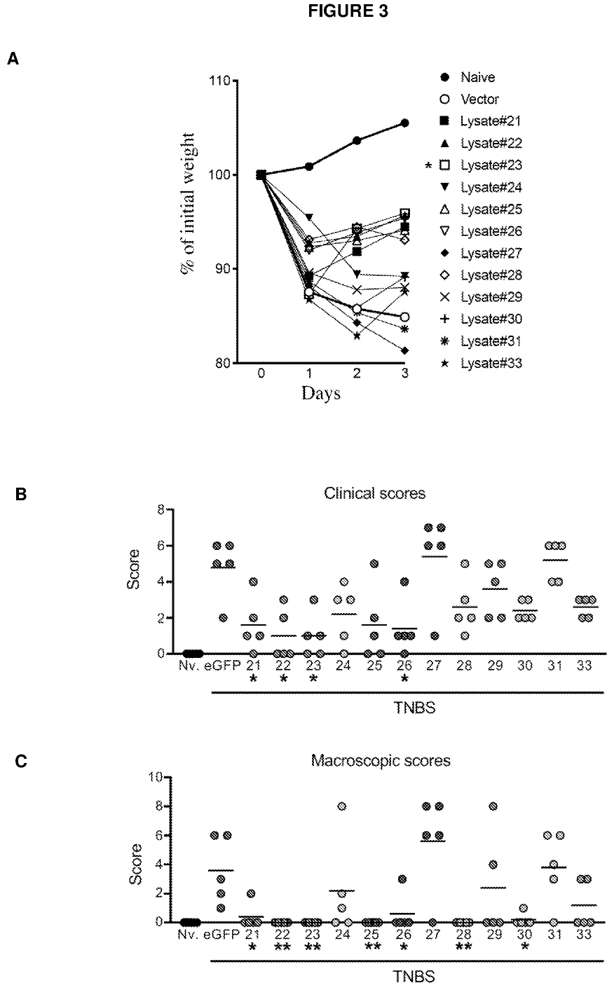

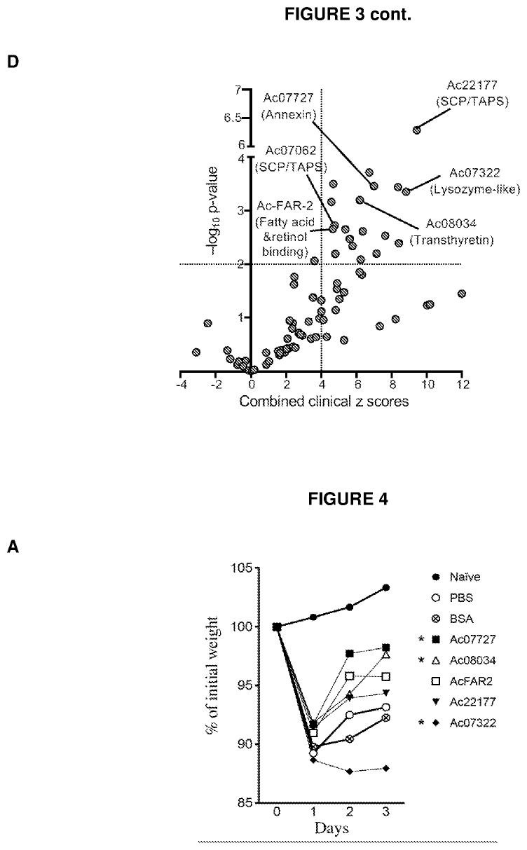

The results of the following lysates are presented in FIGS. 3A-C:
Lysate 21, containing SEQ ID NO:18 described herein.
Lysate 22, containing SEQ ID NO:7 decsribed herein.
Lysate 23, containing SEQ ID NO:3 described herein.
Lysate 28, containing SEQ ID NO:20 described herein.

The following lysates were also compared for comparison:
Lysate 24, containing SEQ ID NO:74 shown in Table 7.
Lysate 25, containing SEQ ID NO:75 shown in Table 7.
Lysate 26, containing SEQ ID NO:76 shown in Table 7.
Lysate 27, containing SEQ ID NO:77 shown in Table 7.
Lysate 29, containing SEQ ID NO:78 shown in Table 7.
Lysate 30, containing SEQ ID NO:79 shown in Table 7.
Lysate 31, containing SEQ ID NO:80 shown in Table 7.
Lysate 32, containing SEQ ID NO:81 shown in Table 7.
Lysate 33, containing SEQ ID NO:82 shown in Table 7.

TABLE 7

| SEQ | | |
|---|---|---|
| ID | Lysate | Amino acid sequence of protein |
| 74 | 24 | MIECPTMDAELRRKIMDRHRKYQPAARGDFEIPKYDCKLEKI |
| | | AKLYLDEPWTPLSSEYGSIKGLGKRGKSIDENLDEAFKAYEW |
| | | NKLKEAAEGGHGREPLIPEHYGCSLRRRIGGVGMYLRCSDL |
| | | ESGLLKLHGNVISVLTINYSQNNQKEKKKKK |
| 75 | 25 | MLKFLLLPTHRQHRGDRGNTIDQKLDEAFKSWEWKKLKEVR |
| | | PCLLRKRF |
| 76 | 26 | MDKHFSIKIKELLKDLRGKDDLWYDCLLEQEARRKQENPEYN |
| | | LDSFYGTVLVKLNKKDGKITPESLEEAFNPLKEKNGKRLRQIK |
| | | YEKVSRYGCWGKFYPQISSELSVLCVYDWKAPQTTN |
| 77 | 27 | MKDSGASHEAIAAHVEEFINAITDEKNIAQALRAAKGCKKVYG |
| | | VARRMRRDHHDHEHNLKEAMEKYLSWMTPEQQAEIKRIFEE |
| | | EGRPAVYKKVMEEFDSASGEVKEKAAEELKAACKHYVKDYI |
| | | GAENAEKIKEMKDSGASKEAIAAKVDEFIAAISDDKRKETAMK |
| | | ASANCKKIYGVARRLRREHHHEHNLEEAMKNYLT |
| 78 | 29 | MNTFANGEWGKEERKSNPIKKGDSFDIRIRAHDDRFQIIIDQK |
| | | EFKDYEHRLPLTSITHLSIDGDLYLNHVHWGGKYYPVPYESGI |
| | | AAGFQVDKTLLIFGTVEKKGKRFNVNLLRRNGGIALHFNPRF |
| | | DEKAVIRNALAANEWGNEEREGKMPFEKGVGFDLAIKNEAY |
| | | AFQIFVNGERFASFAHRSDPNDIT GLQIQGDIELTGIQIQ |
| 79 | 30 | MKELPQKYYGKFDLDHSENFDEYLEAKGNVRHVAAAIFFEDI |
| | | PLRGTTRFLS |
| 80 | 31 | MVHYKLTYFNGRGLGECARQLFALADQQYEDIRVTHEDFPEI |
| | | KPNLPFGQLPLLNEDGKELAQSNAINRYLARKFGFAGKTPFE |
| | | EALVDSLADQMTDYRVEIKPFVYTAYGHQKFGDLETLKKDV |
| | | MLPARDKFLGFITKFLKNNPSGFLVGDSVTWIDLLLAEHASDI |
| | | QSKVPEYLEGFPEVKAHMEKVRSIPKLKKWIETTPDTHF |
| 81 | 32 | MAPNVEVNKFEDIPEQYRELIPKEVADHLKAITDDEKKILKEVL |
| | | KDYAKYKDENEYLAVLKEKSPSLHEKAKKFHEFIKAKVDALG |
| | | DEPKAFVKKVIAAARKLHAELLAGNKPSLEELKNTVKTYMAE |
| | | FNALSPAAKEDLKKNFPILTSVFTNEKAKALMDKHLQN |
| 82 | 33 | MYNFGSILDHIEGEVQFMSDLAYDFFGQGENWNAGLWAYG |
| | | FTHFPTLPNLKNMKNNYKDFEKELGTMEFTDRDNYLTTSKAI |
| | | EVINRLMDDGSSANCLVFFSAQKNTNILPRLSPIYKKWERIVA |
| | | VGFNDTDLSGVVYGTGVAVRVPFHYLDEHVNKVMDAIMGRY |
| | | VPQTTKRPVTVPTPRPTTPVPPGTGQTSLKGTDMSNFGRRY |
| | | G |

Combined clinical Z-scores were then generated to rank and compare the effects of each of the 78 hookworm protein-containing lysates across all screening experiments, based on the four disease outcomes (weight change, clinical score, macroscopic pathology and colon length). Results are shown in FIG. 3D.

### Mice

Male and female specific pathogen free (SPF) BALB/cArc mice were purchased from the Animal Resources Centre (Perth, Australia) and used at 5-10 weeks of age. The animals were maintained in SPF conditions and rested for minimum 7 days between arrival at our facilities and experiments. All experiments were approved by the James Cook University Animal Ethics Committee, and in compliance with the National Health and Medical Research Council (NHMRC) Australian Code for the Care of Animals for Scientific purposes, and the Queensland Animal Care and Protection Act.

### Testing Proteins in TNBS-Induced Colitis Model

Following anaesthesia with 200 ml of 6.25% ketamine (Ketamil, Provet) and 6.25% xylazine (Xylazil, Provet) in PBS, mice were administered intrarectally with 100 μl of 1.5 mg (5% w/v) 2,4,6-Trinitrobenzenesulfonic acid (TNBS) solution in 50% EtOH. Test proteins were administered intraperitoneally with 200 ml of 100 mg/ml protein in PBS on day −1 of the TNBS colitis protocol. The mice were monitored daily for weight loss and clinical scores (combined from weight loss, piloerection, faecal consistency and mobility, each scored from 0-2). Macroscopic scores on day of euthanasia included colon tissue adhesion, ulceration, bowel wall thickening and mucosal oedema, each scoring from 0-3.

### Statistical Analysis

Data were analysed using GraphPad Prism 9.0. Comparison of data to control populations was performed by Mann-Whitney U-test. Normalised data was compared to 100% by one-sample t-test. Technical errors were removed from data prior to analysis. Sample sizes and statistical analysis methods are indicated in the figure legends.

To assess the performance of lysates in the TNBS colitis model, combined clinical Z-scores were generated. The raw data of the four outcomes were used to calculate a combined Z-score. The Z-score transformation combined the four major outcomes of TNBS colitis—% of starting weight on day 3 (weight change), macroscopic pathology, clinical score and colon length—per mouse within a group of 5 mice treated with the same lysate. The Z-score transformed the raw data into units of standard deviation and showed whether the value of the raw score was below or above the population mean. In this case, the transformation reflected the number of standard deviations of the raw score of a given colitis parameter for a test group of mice was from mean of the whole population in the screen. When the population mean scores of both the test (hookworm protein-containing lysate) and negative control (eGFP only lysate) groups were the same value then the Z-score was zero. A positive Z-score was when the test group mean was higher than the negative control group mean, and a negative Z-score was when the test group score was less than the negative control population mean. The four assessed parameters were summed to produce the combined Z-score value, which was compared to the negative control group of each experiment using a two-tailed student's t-test with two sample unequal variance (heteroscedastic). The determined p-value then allowed each test group to be compared against others across the entire screen.

To assess −Log₁₀ p-value, day 3 colon length and weight loss raw scores from test groups (hookworm protein lysates) were compared to the control (eGFP lysates) group, and significance was determined by a two-sample t-test. Macroscopic score and clinical score raw data scores from test groups were compared to the control group and significance was determined by a Mann-Whitney U test. Resultant p-values were adjusted for multiple testing and transformed the four outcomes to produce the geometric mean of p-value for each experiment. The geometric mean was chosen because it is less likely to be influenced by outliers and therefore would not skew the ranking of overall efficacy. The significance of the geometric mean of test groups compared to the control group was determined.

### Results

As expected, mice treated with lysate containing eGFP alone prior to TNBS administration exhibited rapid weight loss (FIG. 3A), elevated clinical scores (FIG. 3B) and macroscopic pathology scores (FIG. 3C) compared to naïve mice. As shown in FIGS. 3A-C, one protein-containing lysate (lysate 23 containing SEQ ID NO:3) was found to reduce all three disease parameters, while six other lysates (lysates 21, 22, 25, 26, 28 and 30) were found to reduce either clinical score or macroscopic colon pathology.

The combined Z-scores generated for the 78 hookworm protein-containing lysates resulted in an overall difference of means score and a p-value for each protein compared to eGFP control. This was used to rank the proteins (FIG. 3D), which allowed 20 proteins to be idenfitied that displayed significant protection in the TNBS colitis model, namely SEQ ID NOs:1-20. These 20 proteins were tested and evaluated in subsequent studies. The proteins that were subsequently expressed in mammalian cells are annotated with their protein name and Pfam status in FIG. 3D.

### Example 6. Effect of Recombinant Proteins in TNBS-Induced Colitis Model and on Human Immune Cells

Proteins contained in the lysates that performed well in Example 5 were prepared as purified recombinant proteins and evaluated for efficacy in a TNBS colitis model and for bioactivity against gut immune cells from human colitis patients. Protein lysates that performed well in Example 5 were expressed in Expi293F human embryonic kidney cells and purified to provide the recombinant proteins. Five of the proteins (SEQ ID NOs:11, 1, 4, 6 and 8, also referred to as AcFar2, Ac22177, Ac07727, Ac07322 and Ac08034 respectively) were produced and purified in sufficient quantities to test in the TNBS colitis model and to explore the impact of co-culturing the purified proteins ex vivo with colon biopsies from ulcerative colitis patients. Protein SEQ ID NO:9 (Ac07062) was produced in a sufficient amount for ex vivo testing with colon biopsies.

### Recombinant Protein Expression in Human Embryonic Kidney Cells

Cell-free lysate proteins were expressed in Expi293F human embryonic kidney cells (Thermo Fisher). ORFs consisted of the signal peptide from A. caninum Ac-ASP-2 (SEQ ID NO:69) followed by the respective ORF (minus the endogenous signal peptide) and a C-terminal 6-His tag. cDNAs were synthesized with mammalian codon bias by Genscript and cloned into the pcDNA3.1 plasmid (Thermo Fisher) by restriction cloning. Plasmids were purified and introduced into Expi293F cells by lipofection using an ExpiFectamine 293 transfection kit (Thermo Fisher) as per the manufacturer's instructions. Recombinant proteins were purified on an AKTA FPLC by immobilised metal affinity chromatography using His-trap excel nickel column and buffer exchanged into tissue culture grade DPBS using Amicon ultra-15 centrifugal concentrators and quantified using a Bicinchoninic Acid kit (Thermo Fisher). Recombinant proteins were assessed for endotoxin using a Limulus Amoebocyte Assay (Thermo Fisher) and only used if endotoxin levels were less than 0.5 endotoxin units/mg protein. Wherever possible, endotoxin-free plasticware was used

### Mice

Male and female specific pathogen free (SPF) BALB/cArc mice were purchased from the Animal Resources Centre (Perth, Australia) and used at 5-10 weeks of age. The animals were maintained in SPF conditions and rested for minimum 7 days between arrival at our facilities and experiments. All experiments were approved by the James Cook University Animal Ethics Committee, and in compliance with the National Health and Medical Research Council (NHMRC) Australian Code for the Care of Animals for Scientific purposes, and the Queensland Animal Care and Protection Act.

### Testing Proteins in TNBS-Induced Colitis Model

Following anaesthesia with 200 ml of 6.25% ketamine (Ketamil, Provet) and 6.25% xylazine (Xylazil, Provet) in PBS, mice were administered intrarectally with 100 μl of 1.5 mg (5% w/v) 2,4,6-Trinitrobenzenesulfonic acid (TNBS) solution in 50% EtOH. Test proteins were administered intraperitoneally with 200 ml of 100 mg/ml protein in PBS on days −1, +1 and +2 of the TNBS colitis protocol. The mice were monitored daily for weight loss and clinical scores (combined from weight loss, piloerection, faecal consistency and mobility, each scored from 0-2). Macroscopic scores on day of euthanasia included colon tissue adhesion, ulceration, bowel wall thickening and mucosal oedema, each scoring from 0-3.

### Isolation of Cells From Human Colon Biopsies

Samples were obtained at the Gastroenterology unit of the Prince Charles Hospital in Chermside, QLD. The study was approved by the Human Research Ethics Committee at Prince Charles Hospital. Studies with human samples were approved by the James Cook University Human Research Ethics Committee. Twelve clinically-diagnosed patients with ulcerative colitis, who were not currently taking other biologic therapies, were recruited to the study. Fresh colon biopsies taken from non-inflamed regions of gut tissue were collected, and single cell suspensions of intraepithelial and lamina propria lymphocytes were isolated. Up to 10 punch biopsies were collected from the colon and stored in 5% foetal bovine serum (FBS) in PBS on ice until further processing. To isolate immune cells, all biopsies from each patient were pooled and placed in Mg⁺⁺-and Ca⁺⁺-free Hanks' Balanced Salt Solution (HBSS) supplemented with 5 mM EDTA, 1 mM dithiothreitol (DTT), 5% FBS and incubated for 30 minutes at 37° C. in a shaking incubator at 250 rpm. The supernatants were collected through a 70 mm strainer, resuspended in RPMI with 10% FBS and kept on ice until further processing. The remaining tissues were placed in RPMI with 0.2 mg/mI DNAse-I (Merck) and 400 U/ml collagenase type I (Gibco), and incubated for 30 minutes at 37° C. in a shaking incubator at 250 rpm. The samples were then filtered and meshed through a 70 mm cell strainer, resuspended in RPMI with 10% FBS and combined with the fractions set aside after incubation with DTT.

### Human Cytokine Release Assays

A total of 50,000 cells were plated per well in 96-well round bottom plates (Falcon). The cultures were supplemented with 50 mg/ml recombinant protein or CSA, except for Ac-FAR-2 which was added at 10 mg/ml based on previous optimisation. Human T-Activator CD3/CD28 Dynabeads (ThermoFisher) were added according to the manufacturer's recommendation. The cells were incubated at 37° C., 5% CO₂ overnight, and supernatants were collected and cryopreserved at −80° C. until further processing. For cytokine release analysis the supernatants were thawed on ice and analysed using a Legendplex Human Inflammation Panel I kit (Biolegend) as per the manufacturer's recommendation.

### Statistical Analysis

Data were analysed using GraphPad Prism 9.0. Comparison of data to control populations was performed by Mann-Whitney U-test. Normalized data was compared to 100% by one-sample t-test. Sample sizes and statistical analysis methods are indicated in the figure legends.

### Results

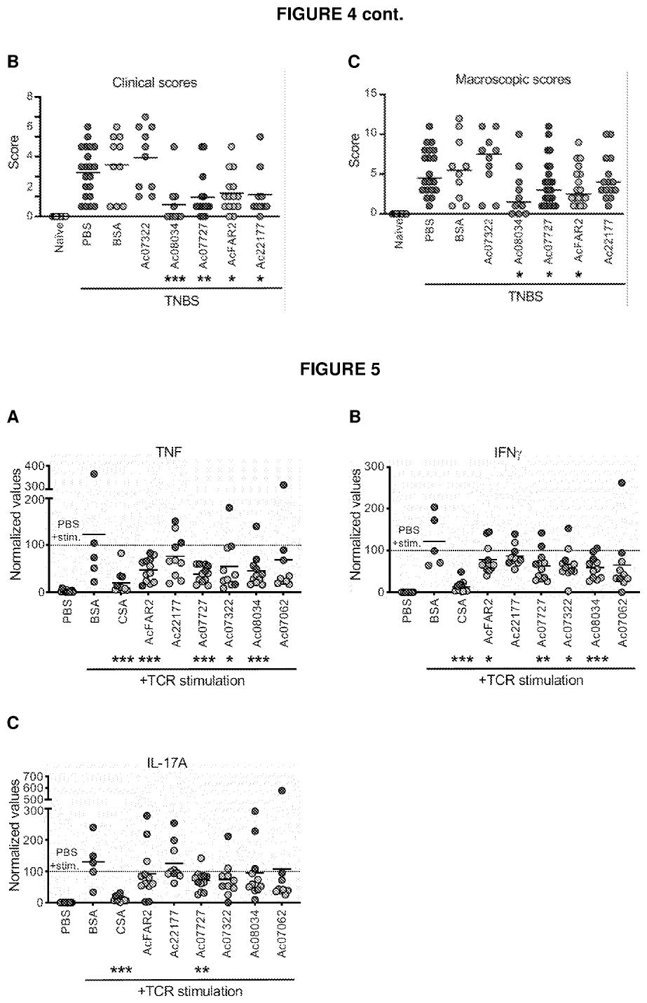

The efficacies of recombinant proteins SEQ ID NOs:11, 1, 4, 6 and 8 (AcFar2, Ac22177, Ac07727, Ac07322 and Ac08034 respectively) were assessed in the TNBS colitis model, compared to PBS vehicle control and an irrelevant control protein (bovine serum albumin, BSA) that was expressed and purified in identical fashion. As expected, PBS vehicle-treated mice displayed weight loss (FIG. 4A), elevated clinical scores (FIG. 4B) and macroscopic pathology scores (FIG. 4C), and this was not affected by co-treatment with the negative control recombinant BSA. As shown in FIGS. 4A-C, Ac08034 and Ac07727 were able to significantly limit weight loss, clinical scores and macroscopic scores, while Ac-FAR-2 and Ac22177 significantly suppressed at least one disease parameter compared to control mice.

The recombinant proteins SEQ ID NOs:11, 1, 4, 6, 8 and 9 (AcFar2, Ac22177, Ac07727, Ac07322, Ac08034 and Ac07062 respectively) were also assessed for bioactivity against gut immune cells from human colitis patients. Analysis of culture supernatants using a bead-based multiplex assay to detect human inflammatory cytokines showed that TCR stimulation in the presence of PBS alone led to secretion of the IBD-relevant cytokines TNF, IFN-γ and IL-17A, and this response was unaffected by co-treatment with BSA control, and was significantly suppressed by CSA (FIGS. 5A-C). As shown in FIGS. 5A-C, Ac07727 was found to significantly reduce the secretion of all three cytokines, while Ac07322 (despite not protecting in TNBS colitis when expressed in Expi293F cells), Ac-FAR-2 and Ac08034 were able to suppress secretion of at least two of the inflammatory cytokines.

1. An isolated, recombinant or synthetic protein comprising an amino acid sequence having at least about 70% sequence identity to any one of SEQ ID NOs:4, 8, 11, 1, 6, 2, 3, 5, 7, 9, 10, 12, 13, 14, 15, 16, 17, 18, 19 and 20, preferably any one of SEQ ID NOs: 4, 8, 11, 1 and 6, more preferably any one of SEQ ID NOs: 4, 8 and 11, or a biologically active fragment or variant thereof.

2. The protein according to claim 1 wherein the amino acid sequence has at least about 80%, at least about 90%, at least about 95%, at least about 98%, or at least about 99% sequence identity to any one of SEQ ID NOs:1-20, preferably any one of SEQ ID NOs: 4, 8, 11, 1 and 6, more preferably any one of SEQ ID NOs: 4, 8 and 11.

3. The protein according to claim 1 or claim 2 wherein the amino acid sequence is any one of SEQ ID NOs:1-20, preferably any one of SEQ ID NOs: 4, 8, 11, 1 and 6, more preferably any one of SEQ ID NOs: 4, 8 and 11.

4. The protein according to any one of claims 1 to 3 wherein the amino acid sequence has at least about 70% sequence identity to any one of SEQ ID NOs:4, 8, 1, 6, 2, 3, 5 and 7, preferably any one of SEQ ID NOs: 4, 8, 1 and 6, more preferably any one of SEQ IDs: 4 and 8.

5. The protein according to any one of claims 1 to 4 wherein the amino acid sequence has at least about 80%, at least about 90%, at least about 95%, at least about 98%, or at least about 99% sequence identity to any one of SEQ ID NOs:1-8, preferably any one of SEQ ID NOs: 4, 8, 1 and 6, more preferably any one of SEQ IDs: 4 and 8.

6. The protein according to any one of claims 1 to 5 wherein the amino acid sequence is any one of SEQ ID NOs:1-8, preferably any one of SEQ ID NOs: 4, 8, 1 and 6, more preferably any one of SEQ IDs: 4 and 8.

7. An isolated, recombinant or synthetic protein comprising an amino acid sequence in which a total of up to about 250 amino acids have been substituted, inserted and/or deleted in any one of SEQ ID NOs:4, 8, 11, 1, 6, 2, 3, 5, 7, 9, 10, 12, 13, 14, 15, 16, 17, 18, 19 and 20, preferably any one of SEQ ID NOs: 4, 8, 11, 1 and 6, more preferably any one of SEQ ID NOs: 4, 8 and 11, or a biologically active fragment or variant thereof.

8. The protein according to claim 7 in which a total of up to about 225, up to about 200, up to about 175, up to about 125, up to about 100, up to about 75, up to about 50, up to about 40, up to about 30, up to about 25, up to about 20, up to about 15 or up to about 10 amino acids have been substituted, inserted and/or deleted in any one of SEQ ID NOs:1-20, preferably any one of SEQ ID NOs: 4, 8, 11, 1 and 6, more preferably any one of SEQ ID NOs: 4, 8 and 1.

9. The protein according to claim 7 or claim 8 wherein the amino acid sequence has a total of up to about 250 amino acids which have been substituted, inserted and/or deleted in any one of SEQ ID NOs:1-8, or a biologically active fragment or variant thereof.

10. The protein according to claim 7 wherein the amino acid sequence has a total of up to about 225, up to about 200, up to about 175, up to about 125, up to about 100, up to about 75, up to about 50, up to about 40, up to about 30, up to about 25, up to about 20, up to about 15 or up to about 10 amino acids which have been substituted, inserted and/or deleted in any one of SEQ ID NOs:4, 8, 1, 6, 2, 3, 5 and 7, preferably any one of SEQ ID NOs: 4, 8, 11, 1 and 6, more preferably any one of SEQ IDs: 4 and 8.

11. A recombinant protein encoded by a nucleotide sequence having at least about 70% sequence identity with any one of SEQ ID NOs:24, 28, 31, 21, 26, 43, 46, 48, 41, 45, 22, 23, 25, 27, 29, 30, 32, 33, 34, 35, 36, 37, 38, 39, 40, 42, 43, 44, 47, 49, 50, 51, 52 and 53, preferably any one of SEQ ID NOs:24, 28, 31, 21 and 26, more preferably any one of SEQ ID NOs: 24, 28 and 31, or a biologically active fragment or variant thereof.

12. The protein according to claim 11 wherein the nucleotide sequence has at least about 80%, at least about 90%, at least about 95%, at least about 98%, or at least about 99% sequence identity to any one of SEQ ID NOs:21-53, preferably any one of SEQ ID NOs:24, 28, 31, 21 and 26, more preferably any one of SEQ ID NOs: 24, 28 and 31.

13. The protein according to claim 11 or claim 12 wherein the nucleotide sequence is any one of SEQ ID NOs:21-53, preferably any one of SEQ ID NOs:24, 28, 31, 21, 26, 43, 46, 48, 41 and 45, more preferably any one of SEQ ID NOs: 24, 28 and 31.

14. The protein according to any one of claims 11 to 13 wherein the nucleotide sequence has at least about 70% sequence identity to any one of SEQ ID NOs:21-28, preferably any one of SEQ ID NOs:24 and 28.

15. The protein according to any one of claims 11 to 14 wherein the nucleotide sequence has at least about 80%, at least about 90%, at least about 95%, at least about 98%, or at least about 99% sequence identity to any one of SEQ ID NOs:21-28, preferably any one of SEQ ID NOs:24 and 28.

16. The protein according to any one of claims 11 to 15 wherein the nucleotide sequence is any one of SEQ ID NOs:21-28, preferably any one of SEQ ID NOs:24 and 28.

17. A pharmaceutical composition comprising the protein according to any one of claims 1 to 16, or a biologically active fragment or variant thereof, and at least one pharmaceutically acceptable carrier.

18. A method for treating or preventing an inflammatory or autoimmune condition comprising administering to a subject in need thereof the protein according to any one of claims 1 to 16, or a biologically active fragment or variant thereof, or the pharmaceutical composition according to claim 17, thereby treating or preventing an inflammatory or autoimmune condition in the subject.

19. A method for the treatment or prevention of an inflammatory or autoimmune condition in a subject comprising the steps of:
identifying a subject having an inflammatory or autoimmune condition; and
administering to the subject in need thereof a therapeutically effective amount of the protein according to any one of claims 1 to 16, or a biologically active fragment or variant thereof, or the pharmaceutical composition according to claim 17
thereby treating or preventing an inflammatory or autoimmune condition in the subject

20. The method according to claim 18 or claim 19 wherein the inflammatory or autoimmune condition is selected from an inflammatory bowel disease, irritable bowel syndrome, type 2 diabetes, asthma, rheumatoid arthritis, lupus, allergy and coeliac disease.

21. Use of the protein according to any one of claims 1 to 16, or a biologically active fragment or variant thereof, in the manufacture of a medicament for treating or preventing an inflammatory or autoimmune condition.

22. The use of claim 21 wherein the inflammatory or autoimmune condition is selected from an inflammatory bowel disease, irritable bowel syndrome, type 2 diabetes, asthma, rheumatoid arthritis, lupus, allergy and coeliac disease.

23. The protein according to any one of claims 1 to 16, ora biologically active fragment or variant thereof, for use in treating or preventing an inflammatory or autoimmune condition.

24. The protein according to claim 23 wherein the inflammatory or autoimmune condition is selected from an inflammatory bowel disease, irritable bowel syndrome, type 2 diabetes, asthma, rheumatoid arthritis, lupus, allergy and coeliac disease.

25. An isolated nucleic acid comprising a nucleotide sequence encoding the protein according to any one of claims 1 to 16 or a biologically active fragment or variant thereof.

26. A vector or expression construct comprising the nucleic acid according to claim 25.

27. A cell comprising the nucleic acid according to claim 25 or the vector or expression construct according to claim 26.

28. A method of producing a protein or a biologically active fragment or variant thereof comprising incubating the cell of claim 27 under conditions that allow it to express the protein or biologically active fragment or variant thereof.


SEQUENCE LISTING (82 sequences)

SEQ ID NO: 1 - protein, 446 aa, Artificial Sequence
  ANCCAN_22177
QAPDYQCWNFGQTNEIRHIYLSEVNKLRQPIGEGKAPNKDGMCPQGKNIYKLFWDCILENYAQEAVDKCGAKPTVPAELSMVWNETTLTTCNPTPSFKKQVNDWWDEVKRVGLGTDAAFKPGLENFAVLANGLATRIGCAQKNCNGKLQMACVVYGKAASNAGQAIYEVGAGCSGDQDCTTYAGSKCLTKKKFCVAGYPTSAVVVPPTVGTTEAMTSSTQSTSTSTSTSTQPEKTTTSEKPSTSATGTSNICPHSPPDANKDLAREELLKVHNEKRAAIAKGEVKMGNGKMARRCPRMKKFVRYNCTLEVAAYEKAKTCPTADPNIENVSCFLSNARNKKLAAREAMDAWFGEISKSNAHMEQATGSQNLLLPALNIRQFARMVWDRNAEMGCGIAECKGSHKYVVVCQYGQGVGKYGDTIYMMGPTCNQCGGPSGGRCTDGAFCP

SEQ ID NO: 2 - protein, 458 aa, Artificial Sequence
  ANCCAN_12564
QNAYCDGGDLLGAEIFEYILDPVNEHRTTLVDGTQKNGLSGQNLPPAKGMTRLDWDCDLEKEAKKIVTSHCFDYVDEPPVMNNRAQVFLSAYADGLPNSPVVMQQLFAQALAEIDTTALEGVTPGATNVIYKSGSVENLLNYFNLMRPANTRMGCAWTKCTNVDGDEFINVYCLLNSQQLKDGDVIYNVGTAGVCSDCPTGTSCDRNTKLCVRTTSVPTTGPAATTTPSTTTTTPAPAQTTTTPPTSPEANFPSGDSSRCSHPLASRMTNTLRSEYERLHNFRRGLLATGQIPRKDGKHLPTASNMQKMTYDCTLEEGAIQYASTCPTRLSELSSRPNVGENFKTFPATRFTFDTAAKKSVTEWWKVIRDVNFFEKVVVFRPFHDGHPISSFTQMAWATSNRLGCSIVNCSTSNRYVGVCRYTQRGNIVNNNVYEVGAVCSACPSGMTSCNRDEGLCF

SEQ ID NO: 3 - protein, 218 aa, Artificial Sequence
  ANCCAN_12569
MSFTTWLQQPHGGGGGGRCAGSATADRMTDTLRDEFERLHNFRRGLLATGQIPRKDGKYLPKASNMRKIIYDCALEEGAIQYASQCPTTHSEQSSRPNVGENFKTFPATRFTFDTAAKKSVTEWWKPIRDVNYFEKVVVFRPFHDGAPISSFTQMGWALSHQVGCSIVMCSASNRYVGVCRYSPRGNIVDNNVYMVGNPCSACHSGATTCSSAEGLCF

SEQ ID NO: 4 - protein, 320 aa, Artificial Sequence
  ANCCAN_07727
MNSPYATIVDNQDFHPALYAEKIDRALRAGDKDTIVKVLTSISNNQRQQLREPYKIKYGKDIIAALDKKFSGDLERTIFALMDTPLDYDVKQLKQAMKGLGTDEAVLIEILCSRTPDQMAAIRIAYEREYKTPLEKDVASDTSGEFKDLLVALVTGSRDGSRNTNDQEAKDDAVRLYADGKAKLVGKDSPSHFLKILATQNQYQLRKVFAAFADLSGSTIEKAIEKEFSGDLQKSYLTIVQAASDKQKFFARQLYNSMKGLGTRDNDLIRVLVTRSEVDLELIKQEFQALYNKPLEDMIKGDTSGQYRDALLAIVVGNRM

SEQ ID NO: 5 - protein, 201 aa, Artificial Sequence
  ANCCAN_00478 (Ac-ASP-2)
NSMRCGNNGMTDEARQKFLDVHNSYRSMVAKGQAKDAISGNAPKAAKMKKMIYDCNVESTAMQNAKKCVFAHSHRKGVGENIWMSTARQMDKAQAAQQASDGWFSELAKYGVGQENKLTTQLWNRGVMIGHYTQMVWQESYKLGCYVEWCSSMTYGVCQYSPQGNMMNSLIYEKGNPCTKDSDCGSNASCSAGEALCVVRG

SEQ ID NO: 6 - protein, 253 aa, Artificial Sequence
  ANCCAN_07322
KAIAPEGVVKAAAGYAYAVDLEVPVPLSGFNCMKKYGYKTVFIRAYDPTGAGRFDTNAVNNIRNANQAGLGTEAYMTPQLHSNKRGSQQFRELYEGLRNAKIQVRTVWVQVTSPVNWGPNTQANIYFINDIVSMAKYYGVTIGFYTNVYDWNQITKGANVEGAMLWYWNVNGAGVNGETPANFDDFRPFAKFTKPTVKQFGQVEHLCGVTVNRDVYTLNNSINLVSSKAEKLDGELIVGTLGGNQMSGLVQSV

SEQ ID NO: 7 - protein, 412 aa, Artificial Sequence
  ANCCAN_26187
TTRFNCRNSLISDEWRQYVLDELNDYRRSLARGEVYDATKNFLPKAKDMNELKWDCNLEEVAYKSLQSLMSTCDVPLFSISRPYQTMQKRFKRKECNITSQAKVLLYGWWKEIKNTTVKYVNGNPSVPTGLEFIVMATATTTGFACTYEFCNNMPRGIIACIYDDKTTDVSVANRIIYQATQNTQEICSSCSKNCIKSLCNVPHTPVTLPTTCSDDKLTQDSNNAALWMHNYYRKLLASGWAKDKKSKSGYALTAKKMLKLEYDCAPAAGGISNGATKTYDLIKDCPKSGNPTPSNGYSMNFLRLKNHTISEQEALEQAIKKWWGVLETKGLGSENTFSDNSEIASFAKMAHDGTEKLACAVQNCKQNGETLVACQYSPAIQDGDKIYETGKVCGGCKALSKKCSDPAGLCE

SEQ ID NO: 8 - protein, 112 aa, Artificial Sequence
  ANCCAN_08034
KMQNVTVKGTTICNKKRMADVTVELWERDTLDPNDLLDSKKTSREGEFLVKGGQNEVGSIEPFLRITHTCNVKKPGCKRITEFDIPKSKIDTVYDMTYVTLDIISAVDKEKC

SEQ ID NO: 9 - protein, 440 aa, Artificial Sequence
  ANCCAN_07062
PNYQCWNFGQQDEIRRDYLVEVNKLRETIATGTAPSKDGRKCPQGKNIYKLNWDCILENYAQKAADECAEPKTVPKKLSFVYNATTLTTCNPIPDIKKQVNNWWSEVERVGLAKNAAFKPKLRNFAVLANGLATRIGCAQKNCNGVLHMVCVVYGKAASSTRQAIYEVGAGCEGNYQCTTYKESKCLTKRKLCEAGYPTSEAVVPPTVSTSKPTITSTEATQPEPQPPKPEPPKPEPPKPETTKPPVTSEHCPKQEHMNTDLAREEFVNLHNQARATIAKGGVKMGDGKMSRPCPRMKKFDIKNYDCKLEEVAYATAAKCQTEVYVQNENWFMASTKATNRKLAAREAVEHWYNEIKSGHMEQKTGSQNLLMPSFKIDHFARMVWDTNTQIGCAAHKCGKIYHVVCRYGPKVGKYGDTIYMMGPTCNQCQGRCIEGAFCP

SEQ ID NO: 10 - protein, 124 aa, Artificial Sequence
  ANCCAN_13497 (Ac-TIMP-1)
CKCEKKPRPPLEKLLCQSQFVTHAKVTKKRIDGYFIYYDLEHKEVYKPKDRSIPIELFSWREKENCGVPDLEEGKEYLIGGKVTDYGDGDLVISVSRCDLLRNWTDVSGEEKKLLGTFKCENQS

SEQ ID NO: 11 - protein, 165 aa, Artificial Sequence
  ANCCAN_10127 (Ac-FAR-2)
APNVEVNKFEDIPEQYRELIPKEVADHLKAITDDEKKILKEVLKDYAKYKDENEYLAVLKEKSPSLHEKAKKFHEFIKAKVDALGDEPKAFVKKVIAAARKLHAELLAGNKPSLEELKNTVKTYMAEFNALSPAAKEDLKKNFPILTSVFTNEKAKALMDKHLQN

SEQ ID NO: 12 - protein, 949 aa, Artificial Sequence
  ANCCAN_01926
MKKAVKWGLLAAVVVAVIAAIVAVVLVLVLKKKDEGPSERVMYFAMHLGDDGALYRNRYEDYKSGMCNLKMNVDNTRSSIKAMKSLTQKYSFILFTDKIETTAPMSSDEAIQKLGSIEPKAHESPFNQEAVLKEFTGRGGDNLLVYYIPCSFDYDSEKVKQFVGEMKKAGLEKKILIVSNTRTEGEIRQLYEQNNVAGSDTENVAEKIIDFGHSESEKTTLEPKTSPKPGTTKTTPQKPSQEPTKPTRPTKPPGPSPNPNGLHCLFVGDLYNFGTRTENYDKEAALFGDIGYDFFEESSISRIGLWAYGHTNYPKKPDLSNMNSNFEDFYSDLNNMEFQNTKDPMNTETAIRVINGLQDDGSIVNCLVFFSARQDTQSLPKLDPQNKKFKRIVAVGYNNTDLSGVVGSRGVAVSVPYHYLDGDVATVLDAIMGRPVTKTTPKTVPTPRPTTSTEVPPMDKSKCLLVGDLYNFGREDEKYDIEADLISKIGYDFLLGSDGDLQLALWAYGYTQFSKTVNDSLKAMRKNYEEFEPDLDKLDFKDVSNPWSSESAIKAINEMYDSQKRVDCLVFFSAQQNPQSLPKLNPKNTKFRKIIAVGFNNVDLRHIVPANGLAFKVPFHYLDEDITPIVKAMHGRYVPKTTVKPTTKKTTTTAPPPPKKLNCLFVGDMYNFGSILDHIEGEVQFMSDLAYDFFGQGENWNAGLWAYGFTHFPTLPNLKNMKNNYKDFEKELGTMEFTDRDNYLTTSKAIEVINRLMDDGSSANCLVFFSAQKNTNILPRLSPIYKKWERIVAVGFNDTDLSGVVYGTGVAVRVPFHYLDEHVNKVMDAIMGRYVPQTTKRPVTVPTPRPTTPVPPGTGQNCLLVTDMSNFGRDMDKYEIELDLLSKVGYDFFEKSNKSVVALWAYGYTDYSKDVAKSLEKLFPNYRQLATELERMKLHETSSPLTTAK

SEQ ID NO: 13 - protein, 219 aa, Artificial Sequence
  ANCCAN_19762
QSLIPFGMEMWYCHSVPRSGPQLRNVFLDLCPNSAFDKEVIEELVLGHINDQRYKLLRGLQLNGPWQGKDKWTPETQGYGKKLPTGKTMNKLEWSCKLEKNAKAALDPKCTDVAPNAPQGKTGLFYKADIDWEVPDITSAAWAWMEQIEKFAVSDDAISDKQVIFKDHDQHHSKTTIWCIRLGKVAATKERTVRMDSHAKMECVRKQTHRRGLHSLMKS

SEQ ID NO: 14 - protein, 195 aa, Artificial Sequence
  ANCCAN_17044
LRQQSVGITGRLMCGDKPAAGVKVKLYDEDDGLDRDDLLDQGFTDARGEFTLKGSERETTNIDPVFKVYHDCDDGIKQSVGITGRLMCGDKPAAGVKVKLYDEDDGLDRDDLLDQGFTDARGEFTLKGSERETTNIDPVFKVYHDCDDGIKPGQRKVKFHIPNQYIYSGGVPRRLFNIGVLNLETIFPDEERDLI

SEQ ID NO: 15 - protein, 165 aa, Artificial Sequence
  ANCCAN_06741
IEFGCKDTLISDKWRETVLNFHNNNRRKISQGQQVSKAGAVMPAAKDMNQLYWDCNIEYNAYLLNCNNAQAKLPTDFGENTASINLGRKCKSPEENTLPILRQWWNEVKLEDLSTPTIDITATPQLKEFAMMANGPTTGFACTYNKKCSDKLLCLYNTQPSKNPT

SEQ ID NO: 16 - protein, 120 aa, Artificial Sequence
  Lysate 74
LCPDLDTTLKKSDFDSANIVLGGNLISVEEKEAEFGLYGRRFNHVVLNMRYSKFYKWKSEFRKDLSPNKLTVPEWTYKRCNMTYTTGNVYIVGCFDLHRCSFMKRLNQFTPNEVSLLNSL

SEQ ID NO: 17 - protein, 257 aa, Artificial Sequence
  ANCCAN_04194 (Ac-NIF)
NEHNLRCPQNGTEMPGFNDSIRLQFLAMHNGYRSKLALGHISITEESESDDDDDFGFLPDFAPRASKMRYLEYDCEAEKSAYMSARNCSDSSSPPEGYDENKYIFENSNNISEAALKAMISWAKEAFNLNKTKEGEGVLYRSNHDISNFANLAWDAREKFGCAVVNCPLGEIDDETNHDGETYATTIHVVCHYPKINKTEGQPIYKVGTPCDDCSEYTKKADNTTSADPVCIPDDGVCFIGSKADYDSKEFYRFREL

SEQ ID NO: 18 - protein, 388 aa, Artificial Sequence
  ANCCAN_12561
MNAITWSCDLEKVTQEGLKSHSCDSNFPPVDPQGRAQLYTSADNEYESTVEEFIASRVDLIDSNALKDVGPNSVKYTDATATSEYSNVMRATTTQIACTKKVCTDINMYVIYCITNQKALTNGEVIYEVGIAGQCTDCPAGTVCDPTSKLCVASGGTVSAGTASTTPATTATAASTTLPTSPEANFPSGASSKCPNSRMTDTLRDEYVRLHNFRRGLLAKGQIPRKDGKYLPMASNMMAITYDCSLEEGAISWAAQCPKASSPQSFRPGLGENCYSFPATRFNFDTAAKKSVTEWWKPIRDVNYFEKVVVFRPFHEGAPISTFTQMGWATSNKLGCSIVKCTTSNRYVGVCRYSPRGNIVNNNVYQVGTPCNACPSGTTCNSSEGLCF

SEQ ID NO: 19 - protein, 195 aa, Artificial Sequence
  ANCCAN_11005
RPVTFVQPQCQPNGYLDKTTIDNGILQPINDERAKVAKHEQQNGMSPTQKLPPATGMTQLKWSCTLEQTAIKALGETCDYDPPANTGGMADFFFSYVTLLKFYVIIIFTYLSIMLVFKPSHFDQGFSRMYEGIQREDFQFFSFFLGCYTPLKVLLFIIIFVAHQYLPLFSCSLALFTTSLSFHTNRCIQSLDSKS

SEQ ID NO: 20 - protein, 400 aa, Artificial Sequence
  ANCCAN_11519
TAFGCNDRLITDEWRAMILKFHNNHRRTVAQGNQAMKGGKMPPGNDINELHWDCNIENYASLHNCNNKIGIMPNFSTISTNIKLTGKPCDINKETKTVLNKLWNEAKNADLKDPKYNDATIKNFGIMAYGNTTGFACTYNKECSDNLLCWYNRKPENGKPLYTSGSDCTKGGCTNSTCVDYLCQLKEYTPTKDFLPRYCHPDVDGLTYEQHLTAQYMMNYYRRLLATGWTRDKSAYAPTATALWSVVYLCASIGKATKALADAVACLDPPFPVARGYTQTHHVINRLNVDPKEALEEAIKAWAEQAKKVDLKPIGGAVFYQDEVEEKASDFAKMITDRNSAFGCGISECKAKSYTLVLCHFNGLFTSRDPIYTVGKPCSRCAKFNKKCEDALGGGLCINK

SEQ ID NO: 21 - DNA, 1137 bp, Artificial Sequence
  ANCCAN_22177
caagctccaggttattatcagtgctggaactttggccagacgaatgagataagacatatctatttatccgaagttaacaaactaagacagccaatcggcgaaggaaaagctccaaacaaggatggcatgtgcccacaggggaagaatatttataagctgttctgggactgtattctggaaaactatgcccaagaagcagtggacaaatgtggtgcaaagccaacagtgccggccgaattatcgatggtttggaacgaaacaacgctaaccacatgcaatccaactccatcattcaagaagcaggtgaatgactggtgggatgaggtgaaaagagtaggactgggcacggatgccgccttcaaaccgggcttggagaattttgccgtgttggcaaatggattggcaacaaggattggctgcgcacaaaagaattgcaacggaaaactccagatggcatgcgtggtttacggcaatagcaacaaccatggctctcatttttatcctaccactatcctgatgctgtgttcaaagaagttcttggataacatctgcccacactctccaccagacgcgaacaaagatctagctagagaagaactcttgaaagtgcacaatgaaaagagagcagcaatcgctaaaggagaagtgaaaatgggaaatggaaagatggctcgtcggtgtccacgaatgaagaaatttgttagatataattgcactcttgaagtagcagcctatgaaaaagctaaaacgtgccctacagcagatccgaatattgagaatgttagctgtttcctgtcgaatgcaagaaataagaagctagccgcgcgcgaggccatggatgcctggtttggagaaattagtaaaagcaatgcccatatggagcaagcgacaggcagccaaaatctgcttctaccagctctcaacataaggcagtttgccaggatggtttgggaccgcaatgctgagatgggctgcggaatcgcagagtgcaaaggcagccataaatatgtcgtggtttgccaatatggacagggtgtgggcaaatatggagataccatctatatgatgggtccaacctgtaaccaatgcggcggcccaagtggcggcagatgtacagacggcgcattctgcccctag

SEQ ID NO: 22 - DNA, 1377 bp, Artificial Sequence
  ANCCAN_12564
caaaatgcatactgtgacggtggagacctgctcggagctgaaatcttcgaatacattctcgatccagtgaacgaacaccgaacgactttggttgatggaactcaaaagaatggtctctccggccaaaacttgccgccagcaaaaggcatgactcgactggattgggactgtgatcttgaaaaggaagctaaaaagattgtaaccagtcattgtttcgactacgttgatgaacctcccgttatgaataacagggctcaagtatttctctcagcatacgcggacggtctgcctaatagtcctgtggtaatgcaacaactcttcgcacaagctctagctgaaattgatactactgccctcgaaggtgtcacccccggcgcgactaacgtcatctacaaatctggcagcgttgaaaatctgttgaactacttcaacctaatgcgacccgcgaatacacgtatgggatgtgcttggacgaagtgtactaatgtcgatggagacgagttcatcaatgtgtactgccttttgaactcacaacaactcaaagacggcgatgtcatctacaatgtgggaacagctggagtttgctccgactgccctactgggacaagttgtgatcgaaatacgaaactatgtgttcgtactacttctgtaccaaccaccggtcccgcggcaacgaccacaccatcaacaaccacaactacacctgcacccgctcaaacaacaacaacacctcctacttctccggaagcgaatttcccttcaggtgacagcagtcgatgttctcatccccttgcaagcaggatgactaatactctccggagtgaatacgagagattacacaatttccgaagaggccttctcgcaaccggacaaattccaagaaaggacgggaaacatctaccaacagcttctaatatgcagaagatgacatatgactgcaccttagaggagggagccatacaatatgctagcacgtgccccacgagactttccgaattgagttccagaccaaatgtgggagagaacttcaaaacattcccagcgacgaggttcacatttgacacagcagctaaaaaatccgttactgagtggtggaaggtgattcgtgacgtgaacttcttcgagaaggtcgtcgtcttccgcccgttccatgacggacatccgatatcctccttcacacagatggcatgggccacaagtaacaggctgggatgctccatcgtcaactgctcgacatccaaccgatacgtgggcgtatgccgatatacgcaaaggggaaatatcgtaaataacaacgtctacgaagttggagccgtttgcagtgcctgtcccagtggaatgacgtcctgcaacagggatgaaggactgtgcttctaa

SEQ ID NO: 23 - DNA, 693 bp, Artificial Sequence
  ANCCAN_12569
atgtcatttacaacgtggctacaacaaccgcaccggtgcgctggcagtgccactgctgacaggatgactgatactcttcgcgatgagttcgagagattgcacaatttccgaagaggcctgctcgcaaccggacaaattccacgaaaggacgggaaatatctaccaaaagcttctaatatgcggaaaattgtagatttattttcacttaacccatttgcggcgatctattatgcaacaattcagatatatgattgcgccttagaggaaggagccatacaatacgcgagccaatgtcccacgacacattccgaacagagctcgagaccgaatgtgggggagaacttcaaaacattcccagcgacaagattcacattcgacacagcagctaagaagtctgttactgaatggtggaagccgattcgtgacgtgaactacttcgaaaaggtcgtcgtcttccgtcccttccatgacggagcaccgatatcctctttcacacagatgggatgggccttaagccaccaggtggggtgctccatcgtcatgtgctcggcgtccaaccgctacgtaggcgtgtgccgatattcgccaagaggcaacatcgtggataataacgtctacatggtaggaaacccctgtagtgcctgccccagcggagcgaccacctgcagcagcgccgaagggctgtgcttttag

SEQ ID NO: 24 - DNA, 1080 bp, Artificial Sequence
  ANCCAN_07727
atgaactctccatacgctacgatcgtcgacaaccaggatttccatcctgccctgtacgcagagaaaatcgaccgtgctctgcgagcaggcgacaaggacaccatcgtgaaggtgctcacctcgatcagcaacaaccagcgacagcagctgcgtgagccatacaaaatcaaatatggcaaggacatcatcgctgcgctcgacaagaagttcagtggcgatctcgagaggaccatcttcgctcttatggatactccacttgactacgacgtcaagcagctgaagcaggctatgaagggccttggcactgacgaggctgtgctgatcgaaattctctgctcccgaactcccgaccagatggctgctatccgaattgcctacgaaagagagtacaaaacgccactggagaaagacgttggttctacattccattcagttcgagagaaaatgaaagcattgaaaacattagatatggtagcgtcagacacatccggagagttcaaggatctgcttgtcgcacttgtcaccggtagcagggacggcagccgcaacaccaacgatcaggaggccaaagatgacgctgtccgtctctacgcggacggcaaggcgaaattggtcggcaaagactcgccatcccacttcctcaagattcttgccacccaaaatcagtaccagcttaggaaggtaagtgtttctcattgcatggttaatcatcgcgaagtaaatgttggtgttaaggtgttcgctgctttcgcggatctttctggatccaccatcgagaaggcgatcgagaaggagttcagcggtgatctccagaagagttacctcaccatcgtccaagccgcttccgacaagcagaagttcttcgctaggcagctgtacaactcgatgaaggtaggcctgggaacccgcgacaacgacctcatccgtgtgctggttacccgctcggaggtggatcttgagctcatcaagcaggagttccaagcgctttacaacaagccactcgaagacatgatcaagggagacacctctggtcagtaccgagacgctcttctcgccatcgtggtcggaaaccgcatgtag

SEQ ID NO: 25 - DNA, 606 bp, Artificial Sequence
  ANCCAN_00478 (Ac-ASP-2)
aattctatgagatgcggaaataatggaatgaccgacgaagcccggcagaaattcctcgacgtgcacaacagttacagatctatggttgccaaaggacaggcaaaggatgcaatttcgggaaatgctccgaaggctgccaaaatgaagaaaatgatctacgactgcaacgtcgaatcaactgcaatgcaaaatgcgaaaaaatgtgttttcgcccattcgcacaggaagggagttggcgaaaatatttggatgtcgactgcgcgtcagatggacaaagcacaagctgctcaacaggctagtgacggttggttcagtgagcttgcgaagtatggtgtaggccaggaaaacaagctaacaacgcagttgtggaacaggggagttatgataggacattacactcagatggtctggcaggagtcctacaaactcggatgttatgtggaatggtgttcatcgatgacctatggtgtctgccagtacagtcctcagggtaatatgatgaactcactcatctacgagaaaggaaacccgtgcacaaaagactctgactgtggctcgaacgccagttgcagcgctggggaggcgctttgcgtcgtgcgtggctag

SEQ ID NO: 26 - DNA, 763 bp, Artificial Sequence
  ANCCAN_07322
aaaagcaatcgctcctgaaggagtagtgaaagcagccgctggctatgcttacgccgtcgatctcgaagtgccagttccactgagcggtttcaactgcatgaaaaaatatggctacaaaactgtattcataagggcatatgatcccacaggtgctggccggttcgatacaaatgctgttaacaacattcgcaacgctaaccaagctggcctgggaaccgaggcgtacatgacaccgcagcttcactcgaacaagagaggcagtcagcaatttcgagaactctatgagggcctcaggaacgccaaaatccaagtgaggaccgtctgggtgcaggtcacctcacccgttaactggggacccaacactcaagcaaatatctatttcatcaacgacatagtatccatggcaaagtactatggtgttacgattggattctatacaaatgtctacgactggaaccagatcacaaaaggcgccaacgtagagggagctatgctctggtactggaacgtgaacggagccggagtgaacggtgagacgccggccaacttcgacgacttccgcccattcgcgaaattcacgaaaccgaccgtgaagcagttcggacaggtggaacatctgtgtggagtcactgtgaacagggacgtctacacgctgaacaactccataaacctcgtctcctcaaaagcagagaaattggacggagagctgattgttggcacccttggaggtaatcagatgtcgggtcttgtacaaagtgtgtaa

SEQ ID NO: 27 - DNA, 1275 bp, Artificial Sequence
  ANCCAN_26187
gctgtggttcccgatgtactcgtcactgtcgaagcaacgacaagattcaactgcagaaactcgctgatttctgacgagtggaggcaatatgttttagacgaacttaatgactatcggagatctctcgcacgtggtgaagtatacgacgcgacgaaaaatttcttgcccaaggctaaagacatgaacgaattgaaatgggactgcaatttggaagaggtggcatataagagcttgcaatcgttaatgagtacttgcgatgtacctcttttcagtatctctcgcccatatcaaaccatgcaaaaacggttcaagaggaaggaatgcaacatcaccagccaagctaaagtattgctatacggatggtggaaggaaattaagaacacaactgtgaagtatgtgaatggcaaccccagtgttccgactggcttggaatttatcgtgatggccaccgccacaacgacaggatttgcctgcacatatgagttttgtaataatatgccgagagggattattgcctgcatttacgacgataaaacaacagacgtgtctgttgctaacagaattatctaccaagcaacgcagaatacgcaagaaatttgtagctcgtgcagcaagaattgcatcaaatccctctgcaatgttccacatactcctgttactcttcctacgacctgctcagatgataagttgacgcaagactcgaataatgcggcactatggatgcacaactattatagaaagctactagcatcgggatgggccaaggataagaagtccaagagtggttatgctttaactgcgaaaaagatgctaaaactagagtacgactgtgctcccgccgctggcggaatttcaaacggtgccacaaagacttatgatttaatcaaggactgtccgaaaagtgggaatcccacaccgtcgaacgggtactccatgaattttttgaggcttaaaaaccacacgatatcggaacaagaggctcttgagcaggcgattaagaaatggtggggagttctcgaaacgaagggccttggctccgaaaacacgttcagcgacaactctgaaattgccagttttgccaaaatggcacacgacggaaccgaaaagcttgcatgtgcggttcagaactgtaagcagaatggagaaacgcttgtcgcgtgccagtattctccagcgatccaagatggcgacaagatttacgagacaggaaaagtatgcggtggttgtaaagctctcagcaagaaatgttcggacccagcgggactttgtgagtaa

SEQ ID NO: 28 - DNA, 337 bp, Artificial Sequence
  ANCCAN_08034
aatgcagaacgtcaccgtcaaggggaccaccatctgcaacaagaagcgaatggccgatgtgacggtggaactgtgggagagagacaccctcgaccccaacgacctcctcgactccaagaagacctctagggaaggcgagttcctcgtgaaaggtggtcagaacgaagtcggctcgattgagccattcctcagaattacacacacctgcaatgtcaagaaaccgggctgcaagagaatcactgagttcgacatcccgaagtcgaagatcgacacggtctacgacatgacctacgtgacgctggatatcatttccgcagtcgataaggagaagtgctag

SEQ ID NO: 29 - DNA, 1323 bp, Artificial Sequence
  ANCCAN_07062
ccaaattaccagtgctggaactttggccagcaggatgagataagacgagactatctagtcgaggttaacaaactaagagagacaatcgccaccggaacagcacccagcaaagatggccgcaagtgtcctcaaggaaagaacatttataagctgaattgggactgtattctggaaaactatgcgcaaaaggctgcggacgaatgcgctgaaccgaagacggttccaaaaaaattgtcatttgtttacaatgcaacaacgctaaccacgtgcaatccaatccctgatatcaagaaacaggtgaacaattggtggagtgaggtagaaagagtaggactggccaaaaatgccgctttcaaacccaagttaaggaatttcgccgtgttggcaaatggattggcaacaaggattggctgtgcacaaaagaactgcaatggagtactgcacatggtgtgtgtggtttatggcaaagcagctagcagcactaggcaagccatatacgaagtgggagcaggctgtgaaggaaactaccagtgtacaacttacaaagaatcgaaatgtctaacaaagagaaaattatgtgaggccggctacccaacaagtgaagcggtagttccaccaaccgtgagcacttctaaaccaacgataacatcgacagaagcaacacaaccagaaccacaaccaccaaaaccagagccaccaaaaccagaaccaccaaaaccagaaacaacaaagcctcctgtaacatctgagcactgcccaaagcaagaacacatgaacactgatctagctagagaagaatttgtgaatctgcacaatcaagctagggcaacaattgctaaaggaggagtcaaaatgggagatggaaagatgtctcgcccgtgtccgcgaatgaagaaatttgatataaagaattatgattgcaaactggaggaagtggcgtacgcaacagccgctaagtgccaaacagaagtttacgttcagaatgaaaactggtttatggcgtcgacgaaagcaacaaatagaaagctagcagctagagaggccgtggaacactggtacaatgaaattaaatcaggccatatggagcaaaaaacagggagtcaaaatttgctcatgcccagcttcaaaatagaccactttgcaaggatggtttgggacaccaatactcaaatcggctgcgcagcccacaagtgcggtaaaatttaccacgtagtgtgccgctatggaccgaaggttggcaaatatggagatacgatttatatgatgggtccaacctgcaaccagtgccaaggtagatgcatagagggagctttttgcccttag

SEQ ID NO: 30 - DNA, 372 bp, Artificial Sequence
  ANCCAN_13497 (Ac-TIMP-1)
tgcaagtgcgaaaagaaacctcgtcctccattggagaaactgctttgccaatcacaatttgttactcacgcgaaagtgacgaagaagagaattgatggttacttcatctattacgacttggagcataaggaagtttataagcccaaagataggagtatcccaatcgaactcttctcatggagggaaaaggaaaattgtggtgttccggatctcgaagaaggcaaagaatacctgataggaggtaaagtgacggattatggcgacggtgatttggtaatttctgtttcacggtgcgaccttctccgaaactggacagacgtctctggagaggagaagaaattgctcggaacgttcaaatgtgaaaatcagtca

SEQ ID NO: 31 - DNA, 498 bp, Artificial Sequence
  ANCCAN_10127 (Ac-FAR-2)
gcccctaacgttgaggtgaacaagtttgaggatattcctgagcagtacagagaactaatccccaaagaagttgccgaccacctcaaagccatcaccgatgacgagaagaagatcttgaaggaggtgctgaaggactacgccaaatacaaggatgaaaacgagtacttggcagtcctgaaggaaaagtctcccagcctacacgagaaggccaagaagttccacgagtttattaaggctaaggtcgacgcacttggggatgaaccgaaggcgttcgtgaagaaagttatcgctgccgctcgaaaactgcatgcagagctccttgccggaaacaagccttctcttgaggaactgaagaacactgtcaagacgtacatggccgaattcaacgccttgtcacccgctgccaaagaggatctgaagaaaaacttccctattctcacctcagtttttacgaacgagaaggcaaaggcgttgatggacaagcacttgcagaactag

SEQ ID NO: 32 - DNA, 2850 bp, Artificial Sequence
  ANCCAN_01926
atgaagaaggcagtcaaatggggacttttggctgctgttgtagtggcggttatcgccgctatagtcgcggttgtcctcgtgctcgtgttgaaaaagaaggacgagggaccttccgaacgagttatgtattttgcaatgcacctcggagatgatggtgcgctgtataggaatagatatgaggattataaaagtggcatgtgcaaccttaaaatgaatgtggataacacccgcagttcgataaaggctatgaagagtttgacacaaaagtacagcttcatattgttcacggacaaaatagaaacgacggcaccaatgagcagcgacgaggcgatccagaaattgggctccattgaaccgaaggcgcatgaatcaccgttcaaccaggaagcggttctgaaagaattcactggccgaggtggagacaatcttcttgtatactacatcccatgcagcttcgattatgactccgaaaaggtgaaacagttcgttggagagatgaagaaggcaggattagaaaaaaagatacttatcgtttcgaatacacgaacagagggcgaaatcaggcagttgtacgagcaaaacaacgtcgctggatccgacacagagaacgtcgcggagaaaatcatcgatttcggtcacagtgaatccgaaaagacaacgctggaaccaaagacgtcgccaaagcctggaaccactaagaccacaccgcaaaagccttcgcaggagcccacgaagccgacaagacccaccaagcctccaggcccttcaccaaaccctaacggactgcactgcctcttcgttggtgatctgtacaatttcggaactagaacagaaaactacgataaggaagcggcactatttggtgacatcggttacgatttcttcgaagagtcgtccatttcgaggatcggtctgtgggcctatggacacacgaactaccccaagaaaccagatcttagcaatatgaacagcaatttcgaagacttctatagtgacttgaataatatggagttccagaacaccaaagatcctatgaatactgaaaccgcaattagagtgatcaacggtctacaagacgatggcagtatcgtcaattgcctcgttttcttctcagcacggcaagatacccagtcccttccaaagctcgacccacagaacaagaagttcaagaggattgtcgcagttggatacaacaacacggatttgagtggagtggtaggatcaagaggagtagccgttagtgtcccttatcactacttagatggtgatgtcgcaacggtcctcgatgcaatcatgggaaggccggtaacgaaaacgacacctaagacggttccgacccctagacctactacttctacagaagtgccaccgatggacaaatcaaaatgtttgctggttggagatctgtacaacttcggtcgcgaggacgagaaatatgacattgaagcggatcttattagcaagatcggttacgacttcctccttggttccgacggcgatctccagcttgcgctttgggcatatggctacacccaattctctaaaacagtgaacgactcactcaaagcgatgcgtaaaaactatgaggaattcgaacctgatctggacaaattggactttaaagatgtttccaatccatggtcatcggagagcgccataaaggctatcaatgagatgtacgacagccagaaacgagtagactgtttggttttcttctcagctcagcagaatcctcagagcttgccgaagctaaatccgaagaacacgaagttcaggaagatcattgcagtgggattcaataatgtggacctgcgacacattgtacctgctaacgggttagccttcaaagttcctttccattacttggatgaagatatcaccccaattgtgaaggcaatgcacggcaggtacgtgccgaaaacgacggtgaaacctaccaccaagaaaacgacaacaactgctccacctcctccaaaaaagcttaactgcctatttgtcggagatatgtacaactttggaagcattctggatcacatcgaaggggaagtacaattcatgtccgatcttgcgtacgatttcttcggccagggtgagaactggaatgctggtctttgggcgtacggattcacacatttccccacacttccaaacctcaagaatatgaaaaacaactacaaagactttgaaaaagaactaggaacaatggaattcaccgaccgcgacaattacctaacaacttcgaaagctattgaggtcatcaacaggctaatggatgacggcagttcagcaaattgtctggtcttcttctctgcacaaaagaacaccaatattctccctcgtctctcgccaatttacaagaaatgggaaagaattgttgcagtagggttcaacgacacagaccttagtggagtggtgtatggaacaggagttgctgtcagggtgcctttccactatttggatgagcatgtgaacaaagtgatggatgcaattatgggaagatatgttccgcagaccactaagagacctgtcactgttcctacaccacgacctacaacgccagtgccgccagggacaggacagaattgcctattggttactgatatgtctaacttcggccgcgatatggataaatacgaaatagaactagacctcttgtccaaagttggatacgacttcttcgaaaaatctaacaaatcagttgtggcactttgggcctatggatacacggactactctaaagacgtggcgaaatcacttgaaaagctgtttccaaactatagacagcttgcaacagaactggagagaatgaagttgcacgagaccagctcacctctgacaacggcgaagtaa

SEQ ID NO: 33 - DNA, 660 bp, Artificial Sequence
  ANCCAN_19762
cagagtttaattccctttggaatggaaatgtggtattgccacagcgtacctcgtagtggaccacaactgagaaacgtctttttagatctttgcccgaatagcgcctttgacaaagaagtgatcgaagaattggttcttggccacatcaacgaccaacgttataaactgctgagaggtctacagctaaatggaccgtggcagggtaaagacaaatggaccccggaaacgcagggatatggaaagaagctgccaacgggcaagacaatgaacaaattggagtggagctgtaaacttgaaaagaacgcgaaagctgcactggatcccaagtgtacagacgttgcacctaacgctcctcaaggaaagactggattgttttacaaggcagacattgactgggaagtgcccgacataacatcggcagcgtgggcatggatggaacaaatcgaaaagtttgctgtaagcgacgacgccatctcagataaacaggttatctttaaagaccatgaccagcaccactcaaagacaacgatatggtgtataaggctgggaaaggtggctgcgacaaaggagagaactgtccgaatggattcacatgcgaaaatggaatgtgtgcgaaagcaaacccataggcgaggccttcactcgcttatgaaatcttga

SEQ ID NO: 34 - DNA, 588 bp, Artificial Sequence
  ANCCAN_17044
ctacgccagcagtcagtcggcattactggacggctcatgtgtggagacaaaccggcagctggggttaaagtaaagctatatgatgaagacgacgggctagatcgtgacgatcttcttgaccaaggattcacagatgctagaggggaattcaccttgaagggttctgagcgcgaaactactaacatcgatccagtattcaaggtataccatgattgtgacgatggcatcaagcagtcagtcggcattactggacggctcatgtgtggagacaaaccggcagctggggttaaagtaaagctatatgatgaagacgacgggctagatcgtgacgatcttcttgaccaaggattcacagatgctagaggggaattcaccttgaagggttctgagcgcgaaactactaacatcgatccagtattcaaggtataccatgattgtgacgatggcatcaagcctggccaacgcaaagtgaagttccatatccccaaccaatacatttattccggaggagtgcccaggcgactattcaatattggcgtgcttaacttggaaactattttccccgatgaagagcgagacttgatctga

SEQ ID NO: 35 - DNA, 495 bp, Artificial Sequence
  ANCCAN_06741
atagagttcggttgcaaagacacgctgatatctgacaaatggcgcgaaaccgtgctgaactttcacaacaacaataggagaaaaatttctcaagggcaacaggtatcgaaagctggggcagtgatgcctgccgcgaaggacatgaaccaactatattgggactgcaacattgaatacaacgcctatctgctcaactgcaacaatgcacaggcaaagttaccaacagatttcggagaaaataccgcgagtatcaatcttggtcgcaaatgcaaaagtcctgaagaaaacacattgccaatcctgcgacagtggtggaacgaagtcaagctcgaggatctgtcaacccctaccatcgacataactgcaacaccgcaattaaaggagtttgcaatgatggctaatgggccgaccactggcttcgcatgcacatacaacaaaaagtgctctgacaaactgctttgtctgtacaatacacagccctccaaaaatcccact

SEQ ID NO: 36 - DNA, 363 bp, Artificial Sequence
  Lysate 74
ttatgtcccgatctggacaccacattaaagaaatccgactttgatagtgcgaacatcgttttgggaggtaatctgatctcggtggaggagaaagaagcagagttcggtctgtatggtcggcgtttcaatcatgtggtactcaacatgcgctactctaagttttacaagtggaagtcagagttccgcaaagatctctcccctaacaaactcaccgttccggaatggacctataaacgctgtaatatgacgtacacaactggaaatgtctacattgtggggtgctttgatctacacagatgctccttcatgaagcgccttaatcagttcacaccgaatgaagttagtctactgaatagcttatag

SEQ ID NO: 37 - DNA, 774 bp, Artificial Sequence
  ANCCAN_04194 (Ac-NIF)
aatgaacacaacctgaggtgcccgcagaatggaacagaaatgcccggtttcaacgactcgattaggcttcaatttttagcaatgcacaatggttacagatcaaaacttgcgctaggtcacatcagcataactgaagaatccgaaagtgacgatgatgacgatttcggttttttacccgatttcgctccaagggcatcgaaaatgagatatctggaatatgactgtgaagctgaaaaaagcgcctacatgtcggctagaaattgctcggacagttcttctccaccagagggctacgatgaaaacaagtatattttcgaaaactcaaacaatatcagtgaagctgctctgaaggccatgatctcgtgggcaaaagaggctttcaacctaaataaaacaaaagaaggagaaggagttctgtaccggtcgaaccacgacatatcaaacttcgctaatctggcttgggacgcgcgtgaaaagtttggttgcgcagttgttaactgccctttgggagaaatcgatgatgaaaccaaccatgatggagaaacctatgcaacaaccatccatgtagtctgccactacccgaaaataaacaaaactgaaggacagccgatttacaaggtagggacaccatgcgacgattgcagtgaatacacaaaaaaagcagacaataccacgtctgcggatccggtgtgtattccggatgacggagtctgctttattggctcgaaagccgattacgatagcaaggagttttatcgattccgagagttatga

SEQ ID NO: 38 - DNA, 498 bp, Artificial Sequence
  ANCCAN_12561
atgactgatactctacgagatgaatacgtcaggctgcacaatttccgaaggggcctactcgcaaaagggcaaattcctagaaaggatgggaagtatctaccaatggcttctaacatgatggcgattacatatgactgcagcttagaagaaggagccatatcgtgggctgcacagtgtcccaaagcaagttcaccgcaaagctttagacccggattgggagagaattgctactcattcccggcgacaaggttcaacttcgacacagcagctaaaaaatctgttactgaatggtggaagccgattcgtgacgtgaattacttcgaaaaggtcgtcgtattccgtccattccatgaaggtgcaccgatatccaccttcacacagatgggatgggctacaagtaacaagttaggatgttcaatcgtgaaatgcacgacgtccaaccgatacgtaggcgtttgccgctattcgccgaggtactcacttattcgttctccctga

SEQ ID NO: 39 - DNA, 204 bp, Artificial Sequence
  ANCCAN_11005
agaccggtgacattcgtccaaccacaatgtcagccaaacggttacctagacaagactacaattgacaacggcattcttcaacccatcaatgatgaaagagcaaaagtggctaaacacgagcaacaaaatggcatgagccctacacagaaactgccaccagcaacgggaatgactcagttggtcagttgttttctgagtggatga

SEQ ID NO: 40 - DNA, 1092 bp, Artificial Sequence
  ANCCAN_11519
atgcagccccctccactctcctgcgttcgctactgcaagcattgggactgcaacatagagaactatgcttcgctgcataattgcaacaacaaaatcggaattatgccaaatttttcgacaatttcaacaaacatcaagctgaccggcaagccttgcgacatcaataaagagacgaaaactgttctaaataaattatggaatgaggcaaagaacgcggatctgaaagatccaaagtacaatgacgctacaataaagaatttcggcataatggcttacgggaacaccactggtttcgcgtgcacctacaacaaagagtgttctgacaatctgctctgttggtacaatcgaaagccggagaacggcaagcctctttacacatctggctccgactgtactaagggtggctgcacaaacagcacctgtgtcgactatctgtgccaactaaaagaatacaccccaactaaggactttcttccaagatactgccacccggacgtcgacggattgacctacgaacaacaccttacagctcagtacatgatgaactattacagaagacttcttgcgaccggatggactcgagacaagagcgcttatgcgccaacagcgacagcgttgtggtcagtggtttacctctgtgcttcaattggaaaagcaaccaaagcattagcagatgcggtagcatgcctggatccaccatttccggtcgcgagaggttacacacagactcatcacgttatcaacagacttaatgttgacccgaaagaagctctcgaagaggcaatcaaagcatgggcggaacaggcaaaaaaagtggatctcaaaccaataggaggggcagttttctaccaggatgaagtcgaagagaaagccagcgattttgcaaaaatgataaccgacagaaattcagcatttggatgcggcatatcggagtgcaaggcgaagagttacacacttgtcttatgtcattttaacgggctctttacttctcgcgatccaatctacactgtcggcaagccatgttcgcgctgcgctaaatttaataagaagtgcgaagatgctcttggcggagggttatgtatcaacaaataa

SEQ ID NO: 41 - DNA, 1416 bp, Artificial Sequence
  ANCCAN_22177
atgctggtgctggtgccactgctggccctgctggccgtgtccgtgcacggccaggcaccagactaccagtgctggaacttcggccagaccaatgagatccggcacatctatctgtctgaggtgaacaagctgagacagcctatcggcgagggcaaggccccaaacaaggatggcatgtgcccccagggcaagaatatctacaagctgttttgggactgtatcctggagaattatgcacaggaggcagtggataagtgcggagcaaagccaaccgtgcctgccgagctgtctatggtgtggaacgagaccacactgaccacatgtaatcctacacccagcttcaagaagcaggtgaacgactggtgggatgaggtgaagagggtgggactgggcaccgacgcagccttcaagccaggcctggagaactttgccgtgctggccaatggcctggccacaagaatcggctgcgcccagaagaactgtaatggcaagctccagatggcctgcgtggtgtacggcaaggcagccagcaatgcaggacaggcaatctatgaagtgggagcaggctgctccggcgaccaggattgtaccacatacgccggctccaagtgcctgaccaagaagaagttttgcgtggcaggctatcccaccagcgccgtggtggtgccccctacagtgggcaccacagaggccatgaccagctccacacagtctacaagcacctccacatctaccagcacacagcccgagaagaccacaacctccgagaagccttccacatctgccaccggcacatccaacatctgtcctcactctccacccgacgccaataaggatctggccagggaggagctgctgaaggtgcacaacgagaagagggcagcaatcgcaaagggagaggtgaagatgggcaatggcaagatggcccggagatgcccacggatgaagaagttcgtgagatacaactgtaccctggaggtggccgcctatgagaaggccaagacctgcccaacagccgaccccaacatcgagaacgtgagctgtttcctgagcaacgccaggaataagaagctggccgcccgcgaggctatggacgcatggtttggcgagatcagcaagtccaacgcacacatggagcaggcaaccggcagccagaacctgctgctgcccgccctgaatatcaggcagttcgcccgcatggtgtgggataggaatgccgagatgggctgcggcatcgccgagtgtaagggctcccacaagtacgtggtggtgtgccagtatggacagggagtgggcaagtacggcgacaccatctatatgatgggccctacatgcaaccagtgtggaggaccatctggcggccggtgcaccgatggcgccttttgtcctcatcaccaccaccaccactgaggatcc

SEQ ID NO: 42 - DNA, 729 bp, Artificial Sequence
  ANCCAN_12569
atgctggtgctggtgcccctgctggccctgctggccgtgtccgtgcacggcatgtctttcaccacatggctccagcagcctcacggcggcggaggaggcggcaggtgcgcaggctccgccacagccgaccgcatgaccgatacactgcgggacgagttcgagagactgcacaactttcggagaggactgctggccaccggacagatcccaaggaaggatggcaagtacctgcccaaggcctctaatatgcgcaagatcatctacgactgcgccctggaggagggagcaatccagtatgcctctcagtgtcctaccacacacagcgagcagagcagccggcccaacgtgggagagaatttcaagacatttcccgccaccagattcacatttgataccgccgccaagaagagcgtgaccgagtggtggaagcccatccgggatgtgaactacttcgagaaggtggtggtgttcagaccctttcacgacggcgcccctatctctagctttacccagatgggatgggcactgagccaccaagtgggctgctccatcgtgatgtgcagcgcctccaatagatacgtgggcgtgtgccggtattcccctcgcggcaacatcgtggacaacaacgtgtacatggtgggcaatccatgctctgcctgtcacagcggagcaaccacatgctcctctgccgagggcctgtgctttcatcaccaccaccaccactgagga

SEQ ID NO: 43 - DNA, 1038 bp, Artificial Sequence
  ANCCAN_07727
atgctggtgctggtgcccctgctggccctgctggccgtgtccgtgcacggcatgaactctccctacgccaccatcgtggacaatcaggatttccaccctgccctgtatgccgagaagatcgatcgggccctgagagccggcgacaaggataccatcgtgaaggtgctgacaagcatctccaacaatcagaggcagcagctgcgcgagccttacaagatcaagtatggcaaggacatcatcgccgccctggataagaagttcagcggcgacctggagcggaccatctttgccctgatggacacaccactggactacgatgtgaagcagctgaagcaggccatgaagggcctgggcaccgatgaggccgtgctgatcgagatcctgtgctccaggacaccagaccagatggccgccatcaggatcgcctacgagcgcgagtataagacccccctggagaaggacgtggccagcgatacatccggcgagtttaaggatctgctggtggccctggtgaccggctctcgggatggcagcagaaacacaaatgaccaggaggccaaggacgatgccgtgaggctgtacgccgatggcaaggccaagctggtgggcaaggactctcccagccacttcctgaagatcctggccacccagaaccagtatcagctgcgcaaggtgttcgccgcctttgccgacctgtccggctctaccatcgagaaggccatcgagaaggagtttagcggcgacctccagaagtcctacctgacaatcgtgcaggccgcctctgacaagcagaagttctttgcccggcagctgtataacagtatgaaaggcctgggcaccagagacaatgatctgatccgggtgctggtgacaagatccgaggtggatctggagctgatcaagcaggagttccaggccctgtacaacaagcctctggaggacatgatcaagggcgatacatctggccagtatagggacgcactgctggccatcgtggtgggaaatcggatgcaccaccaccaccaccactgaggatcc

SEQ ID NO: 44 - DNA, 681 bp, Artificial Sequence
  ANCCAN_00478 (Ac-ASP-2)
atgctggtgctggtgccactgctggccctgctggccgtgtctgtgcacggcaacagcatgagatgtggcaacaatggcatgaccgacgaggccagacagaagttcctggatgtgcacaactcctacaggtctatggtggccaagggccaggccaaggacgccatctccggaaatgcaccaaaggcagccaagatgaagaagatgatctatgattgtaacgtggagtctaccgccatgcagaatgccaagaagtgcgtgttcgcacactctcacaggaagggagtgggagagaatatctggatgagcacagcaaggcagatggacaaggcacaggcagcacagcaggcctccgatggctggttttctgagctggccaagtacggcgtgggccaggagaacaagctgaccacacagctgtggaataggggcgtgatgatcggccactatacccagatggtgtggcaggagagctacaagctgggctgctatgtggagtggtgtagctccatgacctacggcgtgtgccagtatagcccccagggcaacatgatgaacagcctgatctacgagaagggcaacccttgcacaaaggacagcgattgtggctccaatgcctcttgcagcgccggcgaggccctgtgcgtggtgcggggacaccaccaccaccaccactgaggatcc

SEQ ID NO: 45 - DNA, 831 bp, Artificial Sequence
  ANCCAN_07322
atgctggtgctggtgcccctgctggccctgctggccgtgtctgtgcacggcaaggcaatcgcacctgagggagtggtgaaggccgccgccggatacgcctatgccgtggacctggaggtgcccgtgcctctgagcggcttcaattgcatgaagaagtacggctataagaccgtgtttatcagggcctacgacccaaccggagcaggcagattcgatacaaacgccgtgaacaatatcaggaacgcaaatcaggcaggactgggcaccgaggcctacatgacaccccagctgcactctaataagcggggcagccagcagtttagagagctgtatgagggcctgaggaacgccaagatccaggtgcgcaccgtgtgggtgcaggtgacaagcccagtgaactggggccccaatacacaggccaacatctactttatcaatgacatcgtgagcatggccaagtactatggcgtgaccatcggcttctacacaaacgtgtacgattggaaccagatcaccaagggagcaaacgtggagggagcaatgctgtggtattggaacgtgaatggagcaggagtgaatggagagacacctgcaaacttcgacgacttccggcctttcgccaagtttaccaagccaacagtgaagcagttcggacaggtggagcacctgtgcggagtgaccgtgaatcgcgacgtgtatacactgaacaatagcatcaacctggtgagcagcaaggccgagaagctggatggagagctgatcgtgggcaccctgggaggaaatcagatgtccggcctggtgcagtctgtgcaccaccaccaccaccactga

SEQ ID NO: 46 - DNA, 408 bp, Artificial Sequence
  ANCCAN_08034
atgctggtgctggtgcccctgctggccctgctggccgtgtccgtgcacggcaagatgcagaacgtgaccgtgaagggcaccacaatctgcaataagaagcggatggccgacgtgaccgtggagctgtgggagagagacaccctggaccccaacgacctgctggatagcaagaagaccagccgggagggagagttcctggtgaagggaggccagaacgaagtgggctctatcgagccctttctgaggatcacccacacatgcaatgtgaagaagcctggctgtaagcgcatcaccgagttcgacatccctaagtctaagatcgacacagtgtacgatatgacctatgtgacactggacatcatcagcgccgtggataaggagaagtgtcatcatcaccaccaccactga

SEQ ID NO: 47 - DNA, 1407 bp, Artificial Sequence
  ANCCAN_07062
atgctggtgctggtgcctctgctggccctgctggccgtgtctgtgcacggcgcccaggcaccaaactaccagtgctggaatttcggccagcaggacgagatccggagagattatctggtggaggtgaacaagctgagggagacaatcgcaaccggcacagcaccatccaaggacggaaggaagtgccctcagggcaagaacatctacaagctgaattgggactgtatcctggagaattatgcacagaaggccgccgatgagtgcgcagagcctaagaccgtgcccaagaagctgagcttcgtgtacaacgccaccacactgaccacatgcaatcctatcccagacatcaagaagcaggtgaacaattggtggtctgaggtggagagggtgggcctggccaagaacgcagccttcaagcctaagctgcggaactttgccgtgctggccaatggcctggccacaagaatcggctgcgcccagaagaactgtaatggcgtgctgcacatggtgtgcgtggtgtacggcaaggcagccagctccaccaggcaggcaatctatgaagtgggagcaggctgcgagggcaattaccagtgtaccacatataaggagagcaagtgcctgacaaagaggaagctgtgcgaggcaggctaccccacctccgaggcagtggtgccccctacagtgtctaccagcaagcctaccatcacaagcaccgaggcaacccagccagagcctcagccacccaagcccgagcctccaaagcctgagccccctaagccagagaccacaaagccacccgtgacatccgagcactgccccaagcaggagcacatgaacaccgacctggccagggaggagttcgtgaacctgcacaatcaggccagagccaccatcgcaaagggaggagtgaagatgggcgatggcaagatgagcaggccatgtccccgcatgaagaagttcgacatcaagaattacgattgcaagctggaggaggtggcctatgccacagccgccaagtgtcagaccgaggtgtacgtgcagaacgagaattggtttatggcctctacaaaggccaccaacaggaagctggccgccagagaggcagtggagcactggtataatgagatcaagagcggccacatggagcagaagaccggctcccagaacctgctgatgccatctttcaagatcgaccactttgcccggatggtgtgggatacaaatacccagatcggctgcgccgcccacaagtgtggcaagatctaccacgtggtgtgccggtatggcccaaaagtgggcaagtacggcgacacaatctatatgatgggccccacctgcaaccagtgtcagggcaggtgcatcgagggcgccttttgtccccaccaccaccaccaccactgaggatcc

SEQ ID NO: 48 - DNA, 615 bp, Artificial Sequence
  ANCCAN_10127 (Ac-FAR-2)
atgctggtgctggtgcccctgctggccctgctggccgtgtccgtgcacggcgctcctaacgtggaagtgaataagttcgaggacatccccgagcagtacagggagctgatccctaaggaggtggctgatcacctgaaggccatcaccgacgatgagaagaagatcctgaaggaggtgctgaaggactacgctaagtataaggatgagaacgagtatctggccgtgctgaaggagaagtcccctagcctgcacgagaaggctaagaagttccatgagtttatcaaggctaaggtggacgccctgggcgatgagccaaaggccttcgtgaagaaagtgatcgccgctgccaggaagctgcacgctgagctgctggccggcaacaagccctccctggaggagctgaagaataccgtgaagacatacatggctgagtttaacgccctgtctccagctgccaaggaggacctgaagaagaacttccccatcctgacctccgtgtttacaaatgagaaggctaaggccctgatggataagcacctccagaatgaattctgcagatatccagcacagtggcggccgctcgagtctagagggccccatcatcaccatcaccat

SEQ ID NO: 49 - DNA, 2925 bp, Artificial Sequence
  ANCCAN_01926
atgctggtgctggtgcctctgctggccctgctggccgtgagcgtgcacggcatgaagaaggccgtgaagtggggactgctggccgccgtggtggtggccgtgatcgcagcaatcgtggcagtggtgctggtgctggtgctgaagaagaaggatgagggcccatctgagagagtgatgtactttgcaatgcacctgggcgacgatggcgccctgtatcggaatagatacgaggattataagagcggcatgtgcaacctgaagatgaatgtggacaacacccgcagctccatcaaggccatgaagtccctgacacagaagtactctttcatcctgtttaccgataagatcgagaccacagccccaatgtctagcgacgaggccatccagaagctgggctccatcgagcctaaggcccacgagtctccattcaatcaggaggccgtgctgaaggagtttaccggaaggggaggcgacaacctgctggtgtactatatcccctgtagcttcgactacgattccgagaaggtgaagcagtttgtgggcgagatgaagaaggccggcctggagaagaagatcctgatcgtgagcaataccaggacagagggcgagatccgccagctgtatgagcagaacaatgtggccggctccgatacagagaacgtggccgagaagatcatcgacttcggccacagcgagtccgagaagaccacactggagcccaagacctctcctaagccaggcaccacaaagaccacaccccagaagcctagccaggagccaaccaagccaacaaggcctaccaagccccctggcccctcccctaacccaaatggcctgcactgcctgttcgtgggcgatctgtacaattttggcacccggacagagaactatgacaaggaggccgccctgttcggcgacatcggctacgatttctttgaggagtcctctatctccagaatcggcctgtgggcctacggccacacaaattatccaaagaagcccgatctgtccaacatgaacagcaacttcgaggacttttattctgatctgaacaacatggagtttcagaataccaaggaccctatgaacaccgagacagccatcagagtgatcaatggcctccaggacgatggcagcatcgtgaactgcctggtgttcttttccgccagacaggatacacagtctctgcccaagctggaccctcagaacaagaagttcaagaggatcgtggccgtgggctacaacaataccgatctgtctggagtggtgggcagccgcggcgtggccgtgtccgtgccctaccactatctggacggcgatgtggccacagtgctggacgccatcatgggcagacccgtgaccaagaccacacctaagacagtgcccacccctaggccaaccacaagcaccgaggtgccacctatggacaagtccaagtgtctgctggtgggcgacctgtacaacttcggcagggaggacgagaagtatgacatcgaggccgacctgatctctaagatcggctacgatttcctgctgggcagcgacggcgacctccagctggccctgtgggcctacggctatacacagtttagcaagaccgtgaatgactccctgaaggccatgcggaagaactacgaggagttcgagccagacctggataagctggactttaaggatgtgagcaatccctggagcagcgagtccgccatcaaggccatcaacgagatgtatgatagccagaagagagtggactgtctggtgttcttttctgcccagcagaatccacagagcctgcccaagctgaatcctaagaacaccaagttccgcaagatcatcgccgtgggctttaacaatgtggacctgaggcacatcgtgccagcaaacggcctggccttcaaggtgccttttcactacctggacgaggacatcacccccatcgtgaaggccatgcacggccgctatgtgccaaagaccacagtgaagcccaccacaaagaagaccacaaccacagcccctccaccccctaagaagctgaattgtctgttcgtgggcgatatgtacaactttggcagcatcctggaccacatcgagggcgaggtgcagttcatgtccgatctggcctacgacttctttggccagggcgagaattggaacgccggcctgtgggcctatggcttcacacactttcctaccctgccaaatctgaagaacatgaagaacaactacaaggacttcgagaaggagctgggcacaatggagtttaccgaccgcgataactatctgaccacaagcaaggccatcgaagtgatcaaccggctgatggacgatggctctagcgccaattgtctggtgttcttttccgcccagaagaataccaacatcctgccaagactgtctcccatctacaagaagtgggaaagaatcgtggccgtgggcttcaatgacacagatctgtccggagtggtgtacggcaccggagtggccgtgagggtgccctttcactatctggatgagcacgtgaacaaagtgatggacgccatcatgggccggtatgtgcctcagaccacaaagagaccagtcacagtgcccactcctaggccaaccacacctgtgccacccggcacaggccagaattgtctgctggtgaccgacatgtccaactttggccgcgacatggataagtacgagatcgagctggatctgctgtctaaagtgggctatgacttctttgagaagtctaataagagcgtggtggccctgtgggcctacggctacaccgattactctaaggacgtggccaagagcctggagaagctgttccctaactatcgccagctggccacagagctggagcggatgaagctgcacgagacctcctctccactgaccacagccaagcaccaccaccaccaccactgaggatcc

SEQ ID NO: 50 - DNA, 729 bp, Artificial Sequence
  ANCCAN_19762
atgctggtgctggtgcccctgctggccctgctggccgtgtccgtgcacggccagtctctgatccctttcggcatggagatgtggtactgccacagcgtgccaaggtccggaccacagctgaggaacgtgttcctggacctgtgccctaatagcgcctttgataaggaagtgatcgaggagctggtgctgggccacatcaacgaccagcggtacaagctgctgagaggactccagctgaacggaccttggcagggcaaggataagtggaccccagagacacagggctatggcaagaagctgccaaccggcaagacaatgaacaagctggagtggtcctgcaagctggagaagaatgccaaggccgccctggaccccaagtgtaccgatgtggcaccaaacgcacctcagggcaagacaggcctgttttataaggccgacatcgattgggaggtgcccgacatcacctctgccgcctgggcatggatggagcagatcgagaagttcgccgtgagcgacgatgccatctccgataagcaggtcatcttcaaggaccacgatcagcaccactctaagaccacaatctggtgcatcaggctgggcaaggtggcagcaaccaaggagaggacagtgcgcatggacagccacgccaagatggagtgcgtgcggaagcagacacaccggagaggcctgcactctctgatgaagagccaccaccaccaccaccactga

SEQ ID NO: 51 - DNA, 849 bp, Artificial Sequence
  ANCCAN_04194 (Ac-NIF)
atgctggtgctggtgcccctgctggccctgctggccgtgtctgtgcacggcaacgagcacaatctgaggtgcccacagaacggcaccgagatgcctggcttcaatgatagcatccggctccagttcctggccatgcacaacggctacagatccaagctggccctgggccacatctctatcacagaggagagcgagtccgacgatgacgatgacttcggctttctgccagatttcgcccccagggcctccaagatgcgctacctggagtatgactgcgaggccgagaagtctgcctatatgagcgcccggaactgttctgatagctcctctccccctgagggctacgacgagaacaagtatatcttcgagaacagcaacaatatctctgaggccgccctgaaggccatgatctcctgggccaaggaggcctttaacctgaataagaccaaggagggcgagggcgtgctgtacaggagcaatcacgacatctccaacttcgccaatctggcctgggacgcccgcgagaagtttggatgcgcagtggtgaactgtcctctgggcgagatcgatgacgagaccaatcacgacggcgagacatacgccaccacaatccacgtggtgtgccactatccaaagatcaacaagacagagggccagccaatctacaaagtgggcaccccctgcgatgactgtagcgagtatacaaagaaggccgataataccaccagcgccgaccccgtgtgcatcccagatgacggcgtgtgctttatcggctctaaggccgattacgacagcaaggagttctatcggtttagagagctgcaccaccaccaccaccactgaggatcc

SEQ ID NO: 52 - DNA, 1236 bp, Artificial Sequence
  ANCCAN_12561
atgctggtgctggtgcccctgctggccctgctggccgtgtccgtgcacggcatgaacgccatcacatggtcttgcgacctggagaaggtgacccaggagggcctgaagagccactcctgtgactccaacttcccacctgtggacccccagggaagggcccagctgtacacatctgccgataatgagtatgagagcaccgtggaggagtttatcgcctcccgcgtggacctgatcgattctaatgccctgaaggacgtgggcccaaacagcgtgaagtacaccgatgccacagccacctctgagtatagcaacgtgatgagggccaccacaacccagatcgcctgcacaaagaaggtgtgcaccgacatcaatatgtacgtgatctattgcatcacaaatcagaaggccctgaccaacggcgaagtgatctacgaagtgggaatcgcaggacagtgcaccgactgtccagccggcacagtgtgcgatcccacctccaagctgtgcgtggcctccggaggcacagtgtctgccggcaccgccagcacaacccctgcaacaaccgcaacagccgcctccacaaccctgccaacctctcccgaggccaatttcccttctggcgccagctccaagtgcccaaacagccggatgacagacaccctgcgggatgagtatgtgagactgcacaactttcggagaggcctgctggccaagggccagatccccagaaaggacggcaagtacctgcctatggccagcaatatgatggccatcacctatgattgcagcctggaggagggagccatctcctgggcagcccagtgtcctaaggcctctagcccacagtctttccggcccggcctgggagagaattgctacagctttccagccacaagattcaactttgacaccgccgccaagaagtccgtgacagagtggtggaagcccatcagggatgtgaattatttcgagaaggtggtggtgttccgcccatttcacgagggcgcccccatcagcaccttcacccagatgggctgggccaccagcaataagctgggctgctccatcgtgaagtgtacaaccagcaacagatacgtgggcgtgtgccggtattcccccagaggcaatatcgtgaacaataacgtgtaccaagtgggcaccccttgcaatgcctgtccaagcggcacaacctgcaactcctctgagggcctgtgcttccaccaccaccaccaccactga

SEQ ID NO: 53 - DNA, 1272 bp, Artificial Sequence
  ANCCAN_11519
atgctggtgctggtgcctctgctggccctgctggccgtgtctgtgcacggcaccgccttcggctgtaatgaccggctgatcacagatgagtggagagccatgatcctgaagtttcacaacaatcacaggagaaccgtggcacagggaaaccaggcaatgaagggaggcaagatgccccctggcaacgacatcaatgagctgcactgggattgtaacatcgagaattacgcctccctgcacaattgcaacaataagatcggcatcatgcctaatttctctaccatcagcacaaacatcaagctgaccggcaagccatgcgacatcaacaaggagaccaagacagtgctgaacaagctgtggaatgaggccaagaacgccgacctgaaggaccccaagtataatgatgccaccatcaagaacttcggcatcatggcctacggcaataccacaggctttgcctgcacatataataaggagtgtagcgacaacctgctgtgctggtacaatcggaagcctgagaacggcaagccactgtataccagcggctccgactgcacaaagggcggctgtaccaactccacatgcgtggattacctgtgccagctgaaggagtataccccaacaaaggactttctgcccaggtactgtcaccctgacgtggatggcctgacctacgagcagcacctgacagcccagtatatgatgaactactataggcgcctgctggccaccggatggacacgcgataagagcgcctacgccccaaccgcaacagccctgtggagcgtggtgtacctgtgcgcctccatcggcaaggcaaccaaggccctggccgacgcagtggcctgcctggacccccccttccccgtggccaggggctacacccagacacaccacgtgatcaaccgcctgaatgtggaccctaaggaggccctggaggaggccatcaaggcctgggcagagcaggccaagaaggtggacctgaagccaatcggaggagccgtgttttatcaggatgaggtggaggagaaggcctctgacttcgccaagatgatcaccgatagaaatagcgcctttggctgcggcatctccgagtgtaaggccaagtcttacacactggtgctgtgccacttcaacggcctgttcaccagccgggaccccatctatacagtgggcaagccctgctcccggtgtgccaagttcaataagaagtgtgaggacgcactgggaggaggactgtgcatcaacaagcaccaccaccaccaccactga

SEQ ID NO: 54 - protein, 16 aa, Artificial Sequence
  AFS-APN
MLRLALFAVLFACAFS

SEQ ID NO: 55 - protein, 16 aa, Artificial Sequence
  AHA-CKC
MRVAIVFIACFAVAHA

SEQ ID NO: 56 - protein, 16 aa, Artificial Sequence
  SFA-QAP
MRLSVVLAVFVAGSFA

SEQ ID NO: 57 - protein, 21 aa, Artificial Sequence
  AQA-PNY
MRLSLVLAVFVAGNLAQAAQA

SEQ ID NO: 58 - protein, 21 aa, Artificial Sequence
  SQG-QSL
MIAYHFIGFVALLLPLALSQG

SEQ ID NO: 59 - protein, 21 aa, Artificial Sequence
  SSA-TAF
MYLWLLGIFVQLLCNAAVSSA

SEQ ID NO: 60 - protein, 19 aa, Artificial Sequence
  ALC-QNA
MAKFYYIVLVITALPLALC

SEQ ID NO: 61 - protein, 16 aa, Artificial Sequence
  CLA-KAI
MKVLFILPVVLATCLA

SEQ ID NO: 62 - protein, 17 aa, Artificial Sequence
  VHA-KMQ
MRSLCLLLAVVLVAVHA

SEQ ID NO: 63 - protein, 21 aa, Artificial Sequence
  VEA-TTR
MWGILLLLGAVVPDVLVTVEA

SEQ ID NO: 64 - protein, 17 aa, Artificial Sequence
  CLA-LRQ
MRQVLLLLAIVVCSCLA

SEQ ID NO: 65 - protein, 21 aa, Artificial Sequence
  CEG-RPV
MANLNFTALAIASVLLALCEG

SEQ ID NO: 66 - protein, 23 aa, Artificial Sequence
  STA-IEF
MLGSVLGIAVTACILFDVPVSTA

SEQ ID NO: 67 - protein, 17 aa, Artificial Sequence
  TLA-LCP
MKSSMALVALCIATTLA

SEQ ID NO: 68 - protein, 17 aa, Artificial Sequence
  AHS-NEH
MEAYLVVLIAIAGIAHS

SEQ ID NO: 69 - protein, 17 aa, Artificial Sequence
  VHG-NSM
MLVLVPLLALLAVSVHG

SEQ ID NO: 70 - protein, 124 aa, Artificial Sequence
  Lysate 10
MRDDFLDEHNMRRSDVAEGAIPLGPRGKLCPTAARMPKLSYDCNLEKNAYEFARQCSLGGSSESSRQDQGENFHVAPYNGSPTEVGIRAINEWFNEITRKGMNRQMNFTPGIPLVKPKLTSFTQ

SEQ ID NO: 71 - protein, 434 aa, Artificial Sequence
  Lysate 12
TDFQCWNFKSTDTLREHYLKSINNLRKKIADGSAENKSGKCPQGKNIYKLSWDCELELKAQQAVDQCKPNVPEPAGYSQILKKVKSTCDPTKVLKKQIEAWWTKSVKDAGVDNPPNNKQGLEDFAKLANGKATKIGCAQKNCNEQLYVACVINEPAPAVGMPIYEVGAGCNSKDDCTTYLQSKCSNKVCVAGHPGDATTTTSTPATTAPTTPTIPAGPTTAPAPPPTTAAPTTTSTIGSIDNTICPQNQVITDSVRLTFLNTHNGLRSQLAQGQIFMGNGARARPASKMRRMVYNCDAESSARNSAAQCLSSPGSPSGYTENLHVINNNFVDHNSAATQAFNAWWSEINTGYMRQAETERNMYSLSVGIPNFAKMAWETNAHLGCAIVRCGLNTNVVCPYSPKSDGGQIYKMGPFCRRCPDYPGTFCNQGLCSF

SEQ ID NO: 72 - protein, 177 aa, Artificial Sequence
  Lysate 14
LEIVVDNQGSGCSVDRQYREAIDKFHNELRQRIARGDAEKPGQQMYGLVYDCGLEKEADYETRLPGYADLHHHGVTRFSGDYERSALSALREMLKTFSTDKNAMRQVTYPKATRFGCTARLRRNKKTGMRRMDWVCVYDKKPKDGESFEGGKPCNENKDCTYYKGSTCEWNLCYTFF

SEQ ID NO: 73 - protein, 45 aa, Artificial Sequence
  Lysate 20
MEFVCLYDKKPQDGESLEGGSYCKVDQDCTFYKRSYCEDNLCYVQ

SEQ ID NO: 74 - protein, 156 aa, Artificial Sequence
  Lysate 24
MIECPTMDAELRRKIMDRHRKYQPAARGDFEIPKYDCKLEKIAKLYLDEPWTPLSSEYGSIKGLGKRGKSIDENLDEAFKAYEWNKLKEAAEGGHGREPLIPEHYGCSLRRRIGGVGMYLRCSDLESGLLKLHGNVISVLTINYSQNNQKEKKKKK

SEQ ID NO: 75 - protein, 49 aa, Artificial Sequence
  Lysate 25
MLKFLLLPTHRQHRGDRGNTIDQKLDEAFKSWEWKKLKEVRPCLLRKRF

SEQ ID NO: 76 - protein, 121 aa, Artificial Sequence
  Lysate 26
MDKHFSIKIKELLKDLRGKDDLWYDCLLEQEARRKQENPEYNLDSFYGTVLVKLNKKDGKITPESLEEAFNPLKEKNGKRLRQIKYEKVSRYGCWGKFYPQISSELSVLCVYDWKAPQTTN

SEQ ID NO: 77 - protein, 202 aa, Artificial Sequence
  Lysate 27
MKDSGASHEAIAAHVEEFINAITDEKNIAQALRAAKGCKKVYGVARRMRRDHHDHEHNLKEAMEKYLSWMTPEQQAEIKRIFEEEGRPAVYKKVMEEFDSASGEVKEKAAEELKAACKHYVKDYIGAENAEKIKEMKDSGASKEAIAAKVDEFIAAISDDKRKETAMKASANCKKIYGVARRLRREHHHEHNLEEAMKNYLT

SEQ ID NO: 78 - protein, 209 aa, Artificial Sequence
  Lysate 29
MNTFANGEWGKEERKSNPIKKGDSFDIRIRAHDDRFQIIIDQKEFKDYEHRLPLTSITHLSIDGDLYLNHVHWGGKYYPVPYESGIAAGFQVDKTLLIFGTVEKKGKRFNVNLLRRNGGIALHFNPRFDEKAVIRNALAANEWGNEEREGKMPFEKGVGFDLAIKNEAYAFQIFVNGERFASFAHRSDPNDITGLQIQGDIELTGIQIQ

SEQ ID NO: 79 - protein, 52 aa, Artificial Sequence
  Lysate 30
MKELPQKYYGKFDLDHSENFDEYLEAKGNVRHVAAAIFFEDIPLRGTTRFLS

SEQ ID NO: 80 - protein, 207 aa, Artificial Sequence
  Lysate 31
MVHYKLTYFNGRGLGECARQLFALADQQYEDIRVTHEDFPEIKPNLPFGQLPLLNEDGKELAQSNAINRYLARKFGFAGKTPFEEALVDSLADQMTDYRVEIKPFVYTAYGHQKFGDLETLKKDVMLPARDKFLGFITKFLKNNPSGFLVGDSVTWIDLLLAEHASDIQSKVPEYLEGFPEVKAHMEKVRSIPKLKKWIETTPDTHF

SEQ ID NO: 81 - protein, 166 aa, Artificial Sequence
  Lysate 32
MAPNVEVNKFEDIPEQYRELIPKEVADHLKAITDDEKKILKEVLKDYAKYKDENEYLAVLKEKSPSLHEKAKKFHEFIKAKVDALGDEPKAFVKKVIAAARKLHAELLAGNKPSLEELKNTVKTYMAEFNALSPAAKEDLKKNFPILTSVFTNEKAKALMDKHLQN

SEQ ID NO: 82 - protein, 208 aa, Artificial Sequence
  Lysate 33
MYNFGSILDHIEGEVQFMSDLAYDFFGQGENWNAGLWAYGFTHFPTLPNLKNMKNNYKDFEKELGTMEFTDRDNYLTTSKAIEVINRLMDDGSSANCLVFFSAQKNTNILPRLSPIYKKWERIVAVGFNDTDLSGVVYGTGVAVRVPFHYLDEHVNKVMDAIMGRYVPQTTKRPVTVPTPRPTTPVPPGTGQTSLKGTDMSNFGRRYG